(19) United States
(12) Patent Application Publication
(10) Pub. No.: US 2015/0313904 A1
(43) Pub. Date: Nov. 5, 2015

(54) TREATMENT OF CROHN'S DISEASE WITH DELAYED-RELEASE 6-MERCAPTOPURINE

(71) Applicants: Brenda KOLATCH, Jerusalem (IL); Anna HOTOVELY-SALOMON, Modi'in Maccabim Re'ut (IL)

(72) Inventors: Brenda KOLATCH, Jerusalem (IL); Anna HOTOVELY-SALOMON, Modi'in Maccabim Re'ut (IL)

(73) Assignee: Teva Pharmaceuticals Ltd., Petach Tikva (IL)

(21) Appl. No.: 14/701,201

(22) Filed: Apr. 30, 2015

Related U.S. Application Data
(60) Provisional application No. 62/093,210, filed on Dec. 17, 2014.
(60) Provisional application No. 61/988,068, filed on May 2, 2014.

(51) Int. Cl. A61K 31/52; A61K 9/00

(52) U.S. Cl. CPC A61K 31/52; A61K 9/0053

(57) ABSTRACT

Methods of treating patients suffering from Crohn's disease or ulcerative colitis who did not experience a clinical response to previous thiopurine administration, or suffered side effects from previous thiopurine administration, by administering a delayed release pharmaceutical composition comprising 6-mercaptopurine are disclosed. Methods of treating patients suffering from Crohn's disease or ulcerative colitis who are also being administered a steroid, 5-aminosalicylic acid, or an antibiotic by adjunctively administering a delayed release pharmaceutical composition comprising 6-mercaptopurine are also disclosed.

This application claims the benefit or priority of U.S. Provisional Application No. 61/988,068, filed May 2, 2014, and of U.S. Provisional Application No. 62/093,210, filed Dec. 17, 2014, both of which are incorporated herein by reference in their entirety.

Throughout this application, certain publications and patent application publications are referenced. Full citations for the publications may be found immediately preceding the claims. The disclosures of these publications and patent application publications in their entireties are hereby incorporated by reference into this application in order to describe more fully the state of the art to which this invention relates.

### BACKGROUND OF THE INVENTION

Crohn's disease (CD) is an idiopathic and chronic relapsing, remitting inflammatory disease of the gastrointestinal (GI) tract with a prevalence rate of 26-128 per 100,000 in the United States. The peak age of onset of Crohn's disease occurs between the ages of 15 and 30 years, with a second peak between the ages of 60-80 years with no marked difference between sexes. Ashkenazi Jews have a four-fold increased frequency of CD. Living in northern climates, high socioeconomic class, genetic factors, smoking and birth control pills are also associated with an increased risk of CD [Braunwald et al. 2001].

CD is a localized disease, affecting discontinuously any part of the GI tract from the mouth to the anus, but most commonly, the disease is located both in ileum and colon (40%), followed by disease in the small bowel only (30%) and in the colon only (25%). In patients with small intestinal disease, the terminal ileum is involved in 90% of cases. CD causes transmural intestinal damage across the entire thickness of the intestinal wall, with segmental “skip” lesions of patches of diseased bowel interspersed between healthy tissues.

The disease is categorized into three distinct sub-types: active inflammatory, structuring or fibrostenosing, and fistulizing/perforating. Active CD is characterized by focal inflammation and, at times, formation of fistula tracts, which can lead to abscess formation. Subsequently, the bowel wall thickens and becomes narrowed and fibrotic, leading to chronic recurrent bowel obstructions. Only inflammatory CD responds to medical (pharmaceutical) treatment; other types require invasive surgical intervention [Braunwald et al. 2001].

While overall mortality for CD is low, morbidity significantly affects the quality of life for CD patients, who are mostly in their prime years. CD therapy is aimed at reducing inflammation via induction of a remission after a flare-up and maintenance of the remission, once achieved, for as long as possible, allowing patients to normalize their quality of life [Lichtenstein et al. 2004].

Treatments commonly used are the corticosteroids including budesonide [Simms et al. 2001; Summers et al. 1979; Steinhart et al. 2003], immunosuppressant drugs (thiopurines: azathioprine (AZA) and its metabolite, 6-mercaptopurine (6-MP) [Brooke et al. 1969; Present et al. 1980] or the anti-folate, methotrexate); anti-TNFα agents (infliximab) [Hanauer et al. 2002; Targan et al. 1997]aminosalicylates (5-ASA) [Summers et al. 1979] and antibiotics. These therapies have varying degrees of efficacy and safety due to a myriad of side effects; consequently, discontinuation of therapy often occurs [Higgins et al. 2004]. Steroids are used as first-line therapy, while anti-TNFα is used to treat chronic patients exhibiting severe disease or those refractory to steroids.

Both 6-Mercaptopurine (6-MP) and its pro-drug, azathioprine (AZA), have been used in the treatment of CD for over 45 years [Fiser 2006] and are considered relatively safe, as well as efficacious [Kim et al. 1999; Lewis et al. 2001; Francella et al. 2003]. Azathioprine and 6MP interfere with DNA and RNA synthesis and chromosomal replications, leading to diminished proliferation of rapidly dividing cells. They specifically block gene activation of effective lymphocyte clones. In the circulation, killer cell activity is reduced, and in the mucosal lamina propria (LP), the absolute number of plasmocytes is lowered.

Standard 6-MP is typically used as maintenance therapy, rather than for inducing remission because it has a slow onset of action and requires at least 12 weeks and up to several months of administration before its therapeutic effects in CD become apparent. Therefore, it is typically added to initial steroids to ease steroid tapering in remission induction and is continued as maintenance, often for years [Lichtenstein et al. 2006]. Its dose has to be titrated and monitored based on the patient's weight.

Side effects associated with 6-MP use include fever, rash, nausea and headache. Serious adverse events include leucopenia, hepatotoxicity, pancreatitis, severe infections, and bone marrow suppression. When these events occur, 6-MP dosing is lowered, or if necessary, treatment is discontinued.

The importance of mucosal tissue healing in inflammatory bowel disease in general has become clinically relevant in light of recent reports correlating disease activity with a patient's overall risk of developing colorectal cancer. Currently, the severity of mucosal inflammation, as assessed by endoscopy, is considered the gold standard for disease activity in CD, being correlated with reduced hospitalizations, less surgical interventions, and improved patient outcome [Rutgeerts et al. 2006; Pineton de Chambrun et al. 2009; Baert et al. 2010]. Endoscopic and histologic evidence of mucosal healing was associated with a sustained reduction in the expression of inflammatory markers.

Although steroids are typically given as standard treatment for induction of remission in CD, their use has not been correlated with improvement of endoscopically visible lesions and they are ineffective as maintenance therapy [Mantzaris et al. 2009; Sninsky 2001; Rutgeerts 2004; Rutgeerts 2001].

Ulcerative colitis is a chronic disease of the large intestine, also known as the colon, in which the lining of the colon becomes inflamed and develops tiny open sores, or ulcers, that produce pus and mucous. The combination of inflammation and ulceration can cause abdominal discomfort and frequent emptying of the colon. While Crohn's disease can affect any part of the Gastrointestinal (GI) Tract, ulcerative colitis affects only the colon. Additionally, while Crohn's disease can affect all layers of the bowel wall, ulcerative colitis only affects the lining of the colon [CCFA 2015].

The Mayo score has been the main clinical assessment used for determining drug efficacy with regards to ulcerative colitis (UC). It provides a standardized index for measuring disease activity and is the most widely used instrument in clinical trials of UC. The score is composed of four categories (bleeding, stool frequency, physician assessment, and endoscopic appearance) rated from 0-3 that are summed to give a total score that ranges from 0-12 [Travis 2011].

U.S. Patent Application Nos. 2006/0008520 and 2006/0009473, which are incorporated herein by reference in their entireties, disclose delayed release pharmaceutical compositions comprising 6-MP. U.S. Patent Application Publication Nos. 2009/0263482 and 2013/0280328, which are incorporated herein by reference in their entireties, disclose methods of treating certain patients having Crohn's disease with such pharmaceutical compositions. However, neither discloses treatment of patient populations disclosed in the present application.

### SUMMARY OF THE INVENTION

This invention provides a method of treating a human patient suffering from Crohn's disease (CD) or ulcerative colitis (UC) who did not experience a clinical response to previous thiopurine administration, comprising periodically administering to the human patient a delayed release pharmaceutical composition comprising a pharmaceutically acceptable carrier and an amount of 6-mercaptopurine (6-MP) effective to treat the human patient.

The invention also provides a method of treating a human patient suffering from Crohn's disease (CD) or ulcerative colitis (UC) who has experienced an adverse event in response to previous administration of thiopurine, comprising periodically administering to the human patient a delayed release pharmaceutical composition comprising a pharmaceutically acceptable carrier and an amount of 6-mercaptopurine (6-MP) effective to treat the human patient, wherein the adverse event is other than raised liver function test results (LFTs) if the administered thiopurine is 6-MP.

The invention also provides a method of treating a human patient suffering from Crohn's disease (CD) or ulcerative colitis (UC) who is receiving administration of a steroid and who is steroid-dependent, comprising adjunctively periodically administering to the human patient a delayed release pharmaceutical composition comprising a pharmaceutically acceptable carrier and an amount of 6-mercaptopurine (6-MP) effective to treat the human patient.

The invention also provides a method of treating a human patient suffering from Crohn's disease (CD) or ulcerative colitis (UC) who is being administered an antibiotic, comprising adjunctively periodically administering to the human patient a delayed release pharmaceutical composition comprising a pharmaceutically acceptable carrier and an amount of 6-mercaptopurine (6-MP) effective to treat the human patient.

The invention also provides a method of treating a human patient suffering from Crohn's disease (CD) or ulcerative colitis (UC) who is being administered 5-aminosalisylic acid (5-ASA), comprising adjunctively periodically administering to the human patient a delayed release pharmaceutical composition comprising a pharmaceutically acceptable carrier and an amount of 6-mercaptopurine (6-MP) effective to treat the human patient.

### BRIEF DESCRIPTION OF THE FIGURES

FIG. 1: Crohn's Disease Activity Index (CDAI) score by week for delayed-release 6-mercaptopurine (DR 6-MP) and PURINETHOL® treatments, for all subjects who received a subje study number, signed an informed consent form, and received at least one dose of study medication (the “Intent To Treat” (ITT) population).

FIG. 2: CDAI change from baseline by week for DR-6MP and PURINETHOL® treatments (ITT population).

FIG. 3: CDAI relative change (%) from baseline at 8 and 12 weeks for DR-6MP and PURINETHOL® treatments (ITT population).

FIG. 4: CDAI score by week for DR-6MP and PURINETHOL® treatments for all randomized subjects who completed the study according to the protocol (the “Per Protocol” (PP) population).

FIG. 5: CDAI change from baseline for each week for DR-6MP and PURINETHOL® treatments (PP population).

FIG. 6: CDAI relative change (%) from baseline by week for DR-6MP and PURINETHOL® treatments (PP population).

FIG. 7: Frequency of subjects experiencing a response (CDAI score decreases by ≧100 points), remission (CDAI score is <150), or a clinical response (either a response or remission) at week 12 for DR-6MP and PURINETHOL® treatments (ITT population).

FIG. 8: Frequency of subjects experiencing a response, a remission, or a clinical response at week 12 for DR-6MP and PURINETHOL® treatments (PP population).

FIG. 9: The proportion of subjects experiencing a response, a remission, or a clinical response at week 8 for DR-6MP and PURINETHOL® treatments (ITT population).

FIG. 10: The proportion of subjects experiencing remission consecutively at weeks 6 and 8, and weeks 8 and 12, for DR-6MP and PURINETHOL® treatments.

FIG. 11: The change in Inflammatory Bowel Disease Questionnaire (IBDQ) score between baseline and week 12 for DR-6MP and PURINETHOL® treatments (ITT population).

FIG. 12: Correlation between IBDQ changes and CDAI changes at week 12 for DR-6MP and PURINETHOL® treatments.

FIG. 13: Correlation between IBDQ changes and CDAI changes at week 8 for DR-6MP and PURINETHOL® treatments.

FIG. 14: C-reactive protein (CRP) by treatment and visit for DR-6MP and PURINETHOL® treatments (ITT population).

FIG. 15: Erythrocyte sedimentation rate (ESR) levels by treatment and visit (ITT population).

FIG. 16: Change in Interferon-gamma-secreting T cell clones from baseline to week 12 by treatment (ITT population).

FIG. 17: Change in Fluorescence Activated Cell Sorter (FACS) immunology parameters from baseline to week 12 by treatment for DR-6MP and PURINETHOL® treatments (ITT population).

FIG. 18: Relative weight changes from baseline to week 12 for DR-6MP and PURINETHOL® treatments (ITT population).

FIG. 19: Relative Body Mass Index (BMI) changes (median) from baseline to weeks 8 and 12 for DR-6MP and PURINETHOL® treatments (ITT population).

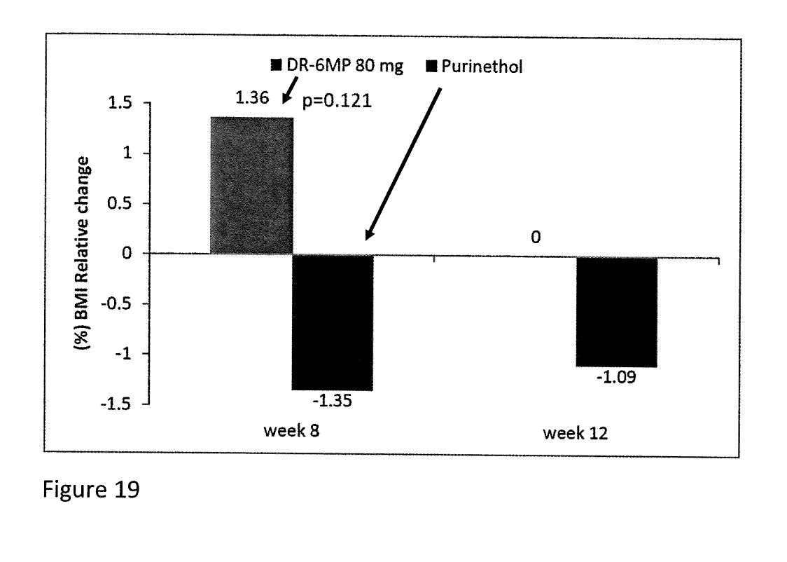

FIG. 20: Mean PURINETHOL® dose by week.

FIG. 21: Number of subjects per PURINETHOL® dose per visit.

FIG. 22: The proportion of subjects with at least one adverse event by treatment group, for DR-6MP and PURINETHOL® treatments.

FIG. 23: The proportion of subjects with at least one drug-related adverse event by treatment group, for DR-6MP and PURINETHOL® treatments.

FIG. 24: Patients (%) with white blood cell (WBC) result within normal range at baseline and week 12 for DR-6MP and PURINETHOL® treatments (ITT population).

FIG. 25: WBC change from baseline to week 12 for DR-6MP and PURINETHOL® treatments (ITT population).

FIG. 26: WBC Changes from Baseline by Treatment, Matching sub-group by baseline weight for DR-6MP and PURINETHOL® treatments.

FIG. 27: Alanine Aminotransferase (ALT) and Aspartate Aminotransferase (AST) levels in one patient during compassionate care treatment with DR-6MP 80 mg.

FIG. 28: Bilirubin and Bilirubin direct levels in the same patient as FIG. 27, during compassionate care treatment with DR-6MP 80 mg.

FIG. 29: ALT and AST levels in a second patient during compassionate care treatment with DR-6MP 80 mg.

FIG. 30: Bilirubin and Bilirubin direct levels in the same patient as FIG. 29 during compassionate care treatment with DR-6MP 80 mg.

FIG. 31: ALT change from baseline to week 12 by treatment for DR-6MP and PURINETHOL® treatments.

FIG. 32: Change in bilirubin direct from baseline to week 12 by treatment for DR-6MP and PURINETHOL® treatments (ITT population).

### DETAILED DESCRIPTION OF THE INVENTION

This invention provides a method of treating a human patient suffering from Crohn's disease (CD) or ulcerative colitis (UC) who did not experience a clinical response to previous thiopurine administration, comprising periodically administering to the human patient a delayed release pharmaceutical composition comprising a pharmaceutically acceptable carrier and an amount of 6-mercaptopurine (6-MP) effective to treat the human patient.

In an embodiment of the instant method, the patient did not experience a clinical response after 4 weeks of previous thiopurine administration. In a further embodiment, the patient did not experience a clinical response after 12 weeks of previous thiopurine administration.

In an embodiment of the instant method, the delayed release pharmaceutical composition is administered daily for a period of time of up to 12 weeks. In a further embodiment, the delayed release pharmaceutical composition is administered daily for a period of time of up to 8 weeks.

In an embodiment of the instant method, the delayed-release pharmaceutical composition is administered daily and the maximal clinical response is achieved 8 weeks from the beginning of administration. In another embodiment, the maximal clinical response is achieved 8 weeks from the beginning of administration.

In an embodiment of the instant method, wherein the patient is suffering from CD and the Crohn's Disease Activity Index (CDAI) score of the patient is about 220 or more before the treatment. In a further embodiment, the CDAI score of the patient is about 220 to about 450 before the treatment.

In an embodiment of the instant method, the patient is suffering from CD and the administration of the delayed release pharmaceutical composition to the patient results in a clinical response.

In an embodiment of the instant method, the patient is suffering from CD and the administration of the delayed release pharmaceutical composition to the patient results in remission of CD.

In an embodiment of the instant method, the patient is suffering from CD and the administration of the delayed release pharmaceutical composition to the patient results in mucosal healing.

In an embodiment of the instant method, the patient is suffering from CD and the administration of the delayed release pharmaceutical composition to the patient results in an improved side effect profile compared to administration of an immediate release formulation of 6-MP.

In an embodiment of the instant method, the patient is suffering from UC and the administration of the delayed release pharmaceutical composition to the patient results in remission of UC.

In an embodiment of the instant method, the patient is suffering from CD and the administration of the delayed release pharmaceutical composition reduces the CDEIS score of the patient by ≧20% relative to baseline after 12 weeks from the beginning of administration.

In an embodiment of the instant method, the patient is suffering from CD and the administration of the delayed release pharmaceutical composition increases the Inflammatory Bowel Disease Questionnaire (IBDQ) score of the patient by ≧10 points relative to baseline after 12 weeks from the beginning of administration. In a further embodiment, the administration of the delayed release pharmaceutical composition increases the Inflammatory Bowel Disease Questionnaire (IBDQ) score of the patient by ≧20 points relative to baseline after 12 weeks from the beginning of administration.

In an embodiment of the instant method, the patient is suffering from CD and the administration of the delayed release pharmaceutical composition decreases the erythrocyte sedimentation rate (ESR) of the patient by ≧1% relative to baseline after 12 weeks from the beginning of administration. In a further embodiment, the administration of the delayed release pharmaceutical composition decreases the erythrocyte sedimentation rate (ESR) of the patient by ≧2% relative to baseline after 12 weeks from the beginning of administration. In a further embodiment, the administration of the delayed release pharmaceutical composition results in a greater decrease in ESR of the patient relative to baseline after 12 weeks from the beginning of administration, compared to administration of an immediate release formulation of 6-mercaptopurine.

In an embodiment of the instant method, the patient is suffering from CD and the administration of the delayed release pharmaceutical composition decreases CD62+ expression of the patient by ≧1.0% relative to baseline after 12 weeks from the beginning of administration.

In an embodiment of the instant method, the patient is suffering from CD and the administration of the delayed release pharmaceutical composition results in weight gain by the patient of ≧0.1% relative to baseline after 12 weeks from the beginning of administration.

In an embodiment of the instant method, the patient is suffering from CD and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from CD and the administration of the delayed release pharmaceutical composition results in decreased incidence of pancreatitis, hepatitis or bone marrow suppression compared to administration of an immediate release formulation of 6-mercaptopurine.

In an embodiment of the instant method, the patient is suffering from UC and the administration of the delayed release pharmaceutical composition results in weight gain by the patient of ≧0.1% relative to baseline after 12 weeks from the beginning of administration.

In an embodiment of the instant method, the patient is suffering from UC and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from UC and the administration of the delayed release pharmaceutical composition results in decreased incidence of pancreatitis, hepatitis or bone marrow suppression compared to administration of an immediate release formulation of 6-mercaptopurine.

In an embodiment of the instant method, the delayed release pharmaceutical composition administered to the patient contains 40 mg to 120 mg of 6-MP. In a further embodiment, the delayed release pharmaceutical composition administered to the patient contains 40 mg to 100 mg of 6-MP. In a further embodiment, the delayed release pharmaceutical composition administered to the patient contains 60 mg to 80 mg of 6-MP. In a further embodiment, the delayed release pharmaceutical composition administered to the patient contains 80 mg of 6-MP. In another embodiment, the delayed release pharmaceutical composition administered to the patient contains 120 mg of 6-MP.

In an embodiment of the instant method, the delayed release pharmaceutical composition is administered once per day. In a further embodiment, the administration is oral administration.

The invention also provides a method of treating a human patient suffering from Crohn's disease (CD) or ulcerative colitis (UC) who has experienced an adverse event in response to previous administration of thiopurine, comprising periodically administering to the human patient a delayed release pharmaceutical composition comprising a pharmaceutically acceptable carrier and an amount of 6-mercaptopurine (6-MP) effective to treat the human patient, wherein the adverse event is other than raised liver function test results (LFTs) if the administered thiopurine is 6-MP.

In an embodiment of the instant method, the delayed release pharmaceutical composition is administered daily for a period of time of up to 12 weeks. In a further embodiment, the delayed release pharmaceutical composition is administered daily for a period of time of up to 8 weeks.

In an embodiment of the instant method, the delayed-release pharmaceutical composition is administered daily and the maximal clinical response is achieved 8 weeks from the beginning of administration. In another embodiment, the maximal clinical response is achieved 8 weeks from the beginning of administration.

In an embodiment of the instant method, the patient is suffering from CD and the Crohn's Disease Activity Index (CDAI) score of the patient is about 220 or more before the treatment. In a further embodiment, the CDAI score of the patient is about 220 to about 450 before the treatment.

In an embodiment of the instant method, the patient is suffering from CD and the administration of the delayed release pharmaceutical composition to the patient results in a clinical response.

In an embodiment of the instant method, the patient is suffering from CD and the administration of the delayed release pharmaceutical composition to the patient results in remission of CD.

In an embodiment of the instant method, the patient is suffering from CD and the administration of the delayed release pharmaceutical composition to the patient results in mucosal healing.

In an embodiment of the instant method, the patient is suffering from CD and the administration of the delayed release pharmaceutical composition to the patient results in an improved side effect profile compared to administration of an immediate release formulation of 6-MP.

In an embodiment of the instant method, the patient is suffering from UC and the administration of the delayed release pharmaceutical composition to the patient results in remission of UC.

In an embodiment of the instant method, the patient is suffering from CD and the administration of the delayed release pharmaceutical composition reduces the CDEIS score of the patient by ≧20% relative to baseline after 12 weeks from the beginning of administration.

In an embodiment of the instant method, the patient is suffering from CD and the administration of the delayed release pharmaceutical composition increases the Inflammatory Bowel Disease Questionnaire (IBDQ) score of the patient by ≧10 points relative to baseline after 12 weeks from the beginning of administration. In a further embodiment, the administration of the delayed release pharmaceutical composition increases the Inflammatory Bowel Disease Questionnaire (IBDQ) score of the patient by ≧20 points relative to baseline after 12 weeks from the beginning of administration.

In an embodiment of the instant method, the patient is suffering from CD and the administration of the delayed release pharmaceutical composition decreases interferon gamma levels of the patient by ≧10% relative to baseline after 12 weeks from the beginning of administration.

In an embodiment of the instant method, the patient is suffering from CD and the administration of the delayed release pharmaceutical composition decreases interferon gamma levels of the patient by ≧25% relative to baseline after 12 weeks from the beginning of administration.

In an embodiment of the instant method, the patient is suffering from CD and the administration of the delayed release pharmaceutical composition decreases C-reactive protein (CRP) levels of the patient by ≧2.5% relative to baseline after 12 weeks from the beginning of administration. In a further embodiment, the administration of the delayed release pharmaceutical composition decreases C-reactive protein (CRP) levels of the patient by ≧5% relative to baseline after 12 weeks from the beginning of administration. In a further embodiment, the administration of the delayed release pharmaceutical composition results in a greater decrease in CRP levels relative to baseline after 12 weeks from the beginning of administration, compared to administration of an immediate release formulation of 6-mercaptopurine.

In an embodiment of the instant method, the patient is suffering from CD and the administration of the delayed release pharmaceutical composition decreases the erythrocyte sedimentation rate (ESR) of the patient by ≧1% relative to baseline after 12 weeks from the beginning of administration. In a further embodiment, the administration of the delayed release pharmaceutical composition decreases the erythrocyte sedimentation rate (ESR) of the patient by ≧2% relative to baseline after 12 weeks from the beginning of administration. In a further embodiment, the administration of the delayed release pharmaceutical composition results in a greater decrease in ESR of the patient relative to baseline after 12 weeks from the beginning of administration, compared to administration of an immediate release formulation of 6-mercaptopurine.

In an embodiment of the instant method, the patient is suffering from CD and the administration of the delayed release pharmaceutical composition decreases CD62+ expression of the patient by ≧1.0% relative to baseline after 12 weeks from the beginning of administration.

In an embodiment of the instant method, the patient is suffering from CD and the administration of the delayed release pharmaceutical composition results in weight gain by the patient of ≧0.1% relative to baseline after 12 weeks from the beginning of administration.

In an embodiment of the instant method, the patient is suffering from CD and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from CD and the administration of the delayed release pharmaceutical composition results in decreased incidence of pancreatitis, hepatitis or bone marrow suppression compared to administration of an immediate release formulation of 6-mercaptopurine.

In an embodiment of the instant method, the patient is suffering from UC and the administration of the delayed release pharmaceutical composition results in weight gain by the patient of ≧0.1% relative to baseline after 12 weeks from the beginning of administration.

In an embodiment of the instant method, the patient is suffering from UC and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from UC and the administration of the delayed release pharmaceutical composition results in decreased incidence of pancreatitis, hepatitis or bone marrow suppression compared to administration of an immediate release formulation of 6-mercaptopurine.

In an embodiment of the instant method, the delayed release pharmaceutical composition administered to the patient contains 40 mg to 120 mg of 6-MP. In a further embodiment, the delayed release pharmaceutical composition administered to the patient contains 40 mg to 100 mg of 6-MP. In a further embodiment, the delayed release pharmaceutical composition administered to the patient contains 60 mg to 80 mg of 6-MP. In a further embodiment, the delayed release pharmaceutical composition administered to the patient contains 80 mg of 6-MP. In another embodiment, the delayed release pharmaceutical composition administered to the patient contains 120 mg of 6-MP.

In an embodiment of the instant method, the delayed release pharmaceutical composition is administered once per day. In a further embodiment, the administration is oral administration.

The invention also provides a method of treating a human patient suffering from Crohn's disease (CD) or ulcerative colitis (UC) who is receiving administration of a steroid and who is steroid-dependent, comprising adjunctively periodically administering to the human patient a delayed release pharmaceutical composition comprising a pharmaceutically acceptable carrier and an amount of 6-mercaptopurine (6-MP) effective to treat the human patient.

In an embodiment of the instant method, the delayed release pharmaceutical composition is administered daily for a period of time of up to 12 weeks. In a further embodiment, the delayed release pharmaceutical composition is administered daily for a period of time of up to 8 weeks.

In an embodiment of the instant method, the delayed-release pharmaceutical composition is administered daily and the maximal clinical response is achieved 8 weeks from the beginning of administration. In another embodiment, the maximal clinical response is achieved 8 weeks from the beginning of administration.

In an embodiment of the instant method, the steroid is an oral steroid. In a further embodiment, the steroid is a low-dose oral steroid. In a further embodiment, the steroid is prednisolone. In a further embodiment, the patient is receiving ≦15 mg of prednisone per day. In another embodiment, the steroid is budesonide. In a further embodiment, the patient is receiving ≦6 mg of budesonide per day.

In an embodiment of the instant method, the patient is suffering from CD and the Crohn's Disease Activity Index (CDAI) score of the patient is about 220 or more before the treatment. In a further embodiment, the CDAI score of the patient is about 220 to about 450 before the treatment.

In an embodiment of the instant method, the patient is suffering from CD and the administration of the delayed release pharmaceutical composition to the patient results in a clinical response.

In an embodiment of the instant method, the patient is suffering from CD and the administration of the delayed release pharmaceutical composition to the patient results in remission of CD.

In an embodiment of the instant method, the patient is suffering from CD and the administration of the delayed release pharmaceutical composition to the patient results in mucosal healing.

In an embodiment of the instant method, the patient is suffering from CD and the administration of the delayed release pharmaceutical composition to the patient results in an improved side effect profile compared to administration of an immediate release formulation of 6-MP.

In an embodiment of the instant method, the patient is suffering from UC and the administration of the delayed release pharmaceutical composition to the patient results in remission of UC.

In an embodiment of the instant method, the patient is suffering from CD and the administration of the delayed release pharmaceutical composition reduces the CDEIS score of the patient by ≧20% relative to baseline after 12 weeks from the beginning of administration.

In an embodiment of the instant method, the patient is suffering from CD and the administration of the delayed release pharmaceutical composition increases the Inflammatory Bowel Disease Questionnaire (IBDQ) score of the patient by ≦20 points relative to baseline after 12 weeks from the beginning of administration. In a further embodiment, the administration of the delayed release pharmaceutical composition increases the Inflammatory Bowel Disease Questionnaire (IBDQ) score of the patient by ≧30 points relative to baseline after 12 weeks from the beginning of administration.

In an embodiment of the instant method, the patient is suffering from CD and the administration of the delayed release pharmaceutical composition decreases interferon gamma levels of the patient by ≧10% relative to baseline after 12 weeks from the beginning of administration. In a further embodiment, the administration of the delayed release pharmaceutical composition decreases interferon gamma levels of the patient by ≧25% relative to baseline after 12 weeks from the beginning of administration.

In an embodiment of the instant method, the patient is suffering from CD and the administration of the delayed release pharmaceutical composition decreases C-reactive protein (CRP) levels of the patient by ≧10% relative to baseline after 12 weeks from the beginning of administration. In a further embodiment, the administration of the delayed release pharmaceutical composition decreases C-reactive protein (CRP) levels of the patient by ≧25% relative to baseline after 12 weeks from the beginning of administration. In a further embodiment, the administration of the delayed release pharmaceutical composition results in a greater decrease in CRP levels relative to baseline after 12 weeks from the beginning of administration, compared to administration of an immediate release formulation of 6-mercaptopurine.

In an embodiment of the instant method, the patient is suffering from CD and the administration of the delayed release pharmaceutical composition decreases the erythrocyte sedimentation rate (ESR) of the patient by ≧1% relative to baseline after 12 weeks from the beginning of administration. In a further embodiment, the administration of the delayed release pharmaceutical composition decreases the erythrocyte sedimentation rate (ESR) of the patient by ≧2% relative to baseline after 12 weeks from the beginning of administration. In a further embodiment, the administration of the delayed release pharmaceutical composition results in a greater decrease in ESR of the patient relative to baseline after 12 weeks from the beginning of administration, compared to administration of an immediate release formulation of 6-mercaptopurine.

In an embodiment of the instant method, the patient is suffering from CD and the administration of the delayed release pharmaceutical composition decreases CD62+ expression of the patient by ≧1.0% relative to baseline after 12 weeks from the beginning of administration.

In an embodiment of the instant method, the patient is suffering from CD and the administration of the delayed release pharmaceutical composition results in weight gain by the patient of ≧0.1% relative to baseline after 12 weeks from the beginning of administration.

In an embodiment of the instant method, the patient is suffering from CD and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from CD and the administration of the delayed release pharmaceutical composition results in decreased incidence of pancreatitis, hepatitis or bone marrow suppression compared to administration of an immediate release formulation of 6-mercaptopurine.

In an embodiment of the instant method, the delayed release pharmaceutical composition administered to the patient contains 40 mg to 120 mg of 6-MP. In a further embodiment, the delayed release pharmaceutical composition administered to the patient contains 40 mg to 100 mg of 6-MP. In a further embodiment, the delayed release pharmaceutical composition administered to the patient contains 60 mg to 80 mg of 6-MP. In a further embodiment, the delayed release pharmaceutical composition administered to the patient contains 80 mg of 6-MP. In another embodiment, the delayed release pharmaceutical composition administered to the patient contains 120 mg of 6-MP.

In an embodiment of the instant method, the patient is suffering from UC and the administration of the delayed release pharmaceutical composition results in weight gain by the patient of ≧0.1% relative to baseline after 12 weeks from the beginning of administration.

In an embodiment of the instant method, the patient is suffering from UC and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from UC and the administration of the delayed release pharmaceutical composition results in decreased incidence of pancreatitis, hepatitis or bone marrow suppression compared to administration of an immediate release formulation of 6-mercaptopurine.

In an embodiment of the instant method, the delayed release pharmaceutical composition is administered once per day. In a further embodiment, the administration is oral administration.

In an embodiment of the instant method, the amount of the delayed release pharmaceutical composition and the amount of the steroid when taken together is more effective to treat the patient than when each agent is administered alone.

The invention also provides a method of treating a human patient suffering from Crohn's disease (CD) or ulcerative colitis (UC) who is being administered an antibiotic, comprising adjunctively periodically administering to the human patient a delayed release phaimaceutical composition comprising a pharmaceutically acceptable carrier and an amount of 6-mercaptopurine (6-MP) effective to treat the human patient.

In an embodiment of the instant method, the patient is suffering from CD and the administration of the delayed release pharmaceutical composition decreases CD62+ expression of the patient by ≧1.0% relative to baseline after 12 weeks from the beginning of administration.

The invention also provides a method of treating a human patient suffering from Crohn's disease (CD) or ulcerative colitis (UC) who is being administered 5-aminosalisylic acid (5-ASA), comprising adjunctively periodically administering to the human patient a delayed release pharmaceutical composition comprising a pharmaceutically acceptable carrier and an amount of 6-mercaptopurine (6-MP) effective to treat the human patient.

In an embodiment of the instant methods, the delayed release pharmaceutical composition administered to the patient contains 40 mg to 120 mg of 6-MP. In a further embodiment, the delayed release pharmaceutical composition administered to the patient contains 40 mg to 100 mg of 6-MP. In a further embodiment, the delayed release pharmaceutical composition administered to the patient contains 60 mg to 80 mg of 6-MP. In a further embodiment, the delayed release pharmaceutical composition administered to the patient contains 80 mg of 6-MP. In another embodiment, the delayed release pharmaceutical composition administered to the patient contains 120 mg of 6-MP.

In an embodiment of the instant methods, the pharmaceutical composition is administered daily for a period of time of up to 12 weeks. In a further embodiment, the delayed release pharmaceutical composition is administered daily for a period of time of up to 8 weeks.

In an embodiment of the instant methods, the delayed release pharmaceutical composition is administered daily and the maximal clinical response is achieved 8 weeks from the beginning of administration. In another embodiment, the maximal clinical response is achieved 8 weeks from the beginning of administration.

In an embodiment of the instant methods, the patient is suffering from CD and the Crohn's Disease Activity Index (CDAI) score of the patient is about 220 or more before the treatment. In a further embodiment, the CDAI score of the patient is about 220 to about 450 before the treatment.

In an embodiment of the instant methods, the patient is suffering from CD and the administration of the delayed release pharmaceutical composition to the patient results in a clinical response.

In an embodiment of the instant methods, the patient is suffering from CD and the administration of the delayed release pharmaceutical composition to the patient results in remission of CD.

In an embodiment of the instant methods, the patient is suffering from CD and the administration of the delayed release pharmaceutical composition to the patient results in mucosal healing.

In an embodiment of the instant methods, the patient is suffering from CD and the administration of the delayed release pharmaceutical composition to the patient results in an improved side effect profile compared to administration of an immediate release formulation of 6-MP.

In an embodiment of the instant methods, the patient is suffering from CD and the administration of the delayed release pharmaceutical composition reduces the CDEIS score of the patient by ≧20% relative to baseline after 12 weeks from the beginning of administration.

In an embodiment of the instant methods, the patient is suffering from CD and the administration of the delayed release pharmaceutical composition increases the Inflammatory Bowel Disease Questionnaire (IBDQ) score of the patient by ≦20 points relative to baseline after 12 weeks from the beginning of administration. In a further embodiment, the administration of the delayed release pharmaceutical composition increases the Inflammatory Bowel Disease Questionnaire (IBDQ) score of the patient by ≦30 points relative to baseline after 12 weeks from the beginning of administration.

In an embodiment of the instant methods, the patient is suffering from CD and the administration of the delayed release pharmaceutical composition decreases interferon gamma levels of the patient by ≧5% relative to baseline after 12 weeks from the beginning of administration. In a further embodiment, the administration of the delayed release pharmaceutical composition decreases interferon gamma levels of the patient by ≧15% relative to baseline after 12 weeks from the beginning of administration.

In an embodiment of the instant methods, the patient is suffering from CD and the administration of the delayed release pharmaceutical composition decreases C-reactive protein (CRP) levels of the patient by ≧5% relative to baseline after 12 weeks from the beginning of administration. In a further embodiment, the administration of the delayed release pharmaceutical composition decreases C-reactive protein (CRP) levels of the patient by ≧15% relative to baseline after 12 weeks from the beginning of administration. In a further embodiment, the administration of the delayed release pharmaceutical composition results in a greater decrease in CRP levels relative to baseline after 12 weeks from the beginning of administration, compared to administration of an immediate release formulation of 6-mercaptopurine.

In an embodiment of the instant methods, the patient is suffering from CD and the administration of the delayed release pharmaceutical composition decreases the erythrocyte sedimentation rate (ESR) of the patient by ≧1% relative to baseline after 12 weeks from the beginning of administration. In a further embodiment, the administration of the delayed release pharmaceutical composition decreases the erythrocyte sedimentation rate (ESR) of the patient by ≧2% relative to baseline after 12 weeks from the beginning of administration. In a further embodiment, the administration of the delayed release pharmaceutical composition results in a greater decrease in ESR of the patient relative to baseline after 12 weeks from the beginning of administration, compared to administration of an immediate release formulation of 6-mercaptopurine.

In an embodiment of the instant methods, the patient is suffering from CD and the administration of the delayed release pharmaceutical composition results in weight gain by the patient of ≦0.1% relative to baseline after 12 weeks from the beginning of administration.

In an embodiment of the instant methods, the patient is suffering from CD and the administration of the delayed release pharmaceutical composition results in a decrease in white blood cell (WBC) count of the patient of ≦11% relative to baseline after 12 weeks from the beginning of administration. In a further embodiment, the administration of the delayed release pharmaceutical composition results in a smaller decrease in WBC count relative to baseline after 12 weeks from the beginning of administration, compared to treatment by an immediate release formulation of 6-mercaptopurine.

In an embodiment of the instant methods, the patient is suffering from CD and the administration of the delayed release pharmaceutical composition results in decreased incidence of pancreatitis, hepatitis or bone marrow suppression, compared to administration of an immediate release formulation of 6-mercaptopurine.

In an embodiment of the instant method, the patient is suffering from UC and the administration of the delayed release pharmaceutical composition results in weight gain by the patient of ≧0.1% relative to baseline after 12 weeks from the beginning of administration.

In an embodiment of the instant method, the patient is suffering from UC and the administration of the delayed release pharmaceutical composition does not result in a decrease in white blood cell (WBC) count of the patient of ≧11% relative to baseline after 12 weeks from the beginning of administration.

In an embodiment of the instant method, the patient is suffering from UC and the administration of the delayed release pharmaceutical composition results in decreased incidence of pancreatitis, hepatitis or bone marrow suppression compared to administration of an immediate release formulation of 6-mercaptopurine.

In an embodiment of the instant methods, the delayed release pharmaceutical composition is administered once per day. In a further embodiment, the administration is oral administration.

In an embodiment of the instant methods, the amount of the delayed release pharmaceutical composition and the amount of the 5-ASA or antibiotic when taken together is more effective to treat the patient than when each agent is administered alone.

The invention also provides a delayed-release pharmaceutical composition comprising 6-mercaptopurine for use in treating a human patient suffering from Crohn's disease (CD) or ulcerative colitis (UC) who did not experience a clinical response to previous thiopurine administration.

The invention also provides a delayed-release pharmaceutical composition comprising 6-mercaptopurine for use in treating a human patient suffering from Crohn's disease (CD) or ulcerative colitis (UC) who has experienced an adverse event in response to previous administration of thiopurine.

The invention also provides a delayed-release pharmaceutical composition comprising 6-mercaptopurine for use in treating a human patient suffering from Crohn's disease (CD) or ulcerative colitis (UC) who is receiving administration of a steroid and who is steroid-dependent.

The invention also provides a delayed-release pharmaceutical composition comprising 6-mercaptopurine for use in treating a human patient suffering from Crohn's disease (CD) or ulcerative colitis (UC) who is being administered an antibiotic.

The invention also provides a delayed-release pharmaceutical composition comprising 6-mercaptopurine for use in treating a human patient suffering from Crohn's disease (CD) or ulcerative colitis (UC) who is being administered 5-aminosalisylic acid (5-ASA).

The invention also provides for use of 6-mercaptopurine for preparation of a medicament for use in treating a human patient suffering from Crohn's disease (CD) or ulcerative colitis (UC) who did not experience a clinical response to previous thiopurine administration, wherein the medicament is a delayed-release medicament.

The invention also provides for use of 6-mercaptopurine for preparation of a medicament for use in treating a human patient suffering from Crohn's disease (CD) or ulcerative colitis (UC) who has experienced an adverse event in response to previous administration of thiopurine, wherein the medicament is a delayed-release medicament.

The invention also provides for use of 6-mercaptopurine for preparation of a medicament for use in treating a human patient suffering from Crohn's disease (CD) or ulcerative colitis (UC) who is receiving administration of a steroid and who is steroid-dependent, wherein the medicament is a delayed-release medicament.

The invention also provides for use of 6-mercaptopurine for preparation of a medicament for use in treating a human patient suffering from Crohn's disease (CD) or ulcerative colitis (UC) who is being administered an antibiotic, wherein the medicament is a delayed-release medicament.

The invention also provides for use of 6-mercaptopurine for preparation of a medicament for use in treating a human patient suffering from Crohn's disease (CD) or ulcerative colitis (UC) who is being administered 5-aminosalisylic acid (5-ASA), wherein the medicament is a delayed-release medicament.

For the foregoing embodiments, each embodiment disclosed herein is contemplated as being applicable to each of the other disclosed embodiment.

All combinations, sub-combinations, and permutations of the various elements of the methods described herein are envisaged and are within the scope of the invention. For example, as treatment of patients who previously experienced adverse events while undergoing administration of a thiopurine is disclosed, and treatment of patients adjunctively receiving steroids is disclosed, treatment of a patient who previously experienced adverse events while undergoing administration of a thiopurine and who is also adjunctively receiving steroids is within the scope of the invention.

By any range disclosed herein, it is meant that all hundredth, tenth and integer unit amounts within the range are specifically disclosed as part of the invention. Thus, for example, 40 mg to 120 mg means that 40.01, 40.02 . . . 40.09; 40.1, 40.2 . . . 40.9; and 41, 42 . . . 119 mg unit amounts are included as embodiments of this invention.

### Terms

As used herein, and unless stated otherwise, each of the following terms shall have the definition set forth below.

The articles “a”, “an” and “the” are non-limiting. For example, “the method” includes the broadest definition of the meaning of the phrase, which can be more than one method.

As used herein, “about” in the context of a numerical value or range means±10% of the numerical value or range recited or claimed.

As used herein, a subject or patient at “baseline” is as subject prior to administration of 6-mercaptopurine in a delayed release or standard release formation in a therapy as described herein.

As used herein, “administering” to a subject means the giving of, dispensing of, or application of medicines, drugs, or remedies to a subject to relieve or cure a pathological condition. Oral administration is an example of administration used in the instant methods.

As used herein, “periodic administration” means repeated/recurrent administration separated by a period of time. The period of time between administrations is preferably consistent from time to time.

Periodic administration can include administration, e.g., once daily, twice daily, three times daily, four times daily, weekly, twice weekly, three times weekly, four times weekly and so on, etc.

As used herein, a “delayed release 6-MP pharmaceutical composition” or a “delayed release pharmaceutical composition comprising 6-MP” refers to a pharmaceutical composition comprising 6-MP where release of 6-MP occurs after passage of the pharmaceutical composition through the stomach.

As used herein, a “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering compounds recited in the instant methods to the subject.

As used herein “Adverse event” or “AE” means any untoward medical occurrence in a clinical trial subject administered a drug. An adverse event can therefore be any unfavorable and unintended sign including an abnormal laboratory finding, symptom, or diseases temporally associated with the use of an investigational medicinal product, whether or not considered related to the investigational medicinal product. Adverse events can be classified as Mild (easily tolerated), Moderate (sufficiently discomforting to interfere with daily activity) or Severe (prevents normal daily activities) Examples of adverse events include fever, rash, nausea and headache. Serious adverse events include leucopenia, hepatotoxicity, pancreatitis, severe infections, and bone marrow suppression.

As used herein, to “treat” or “treating” encompasses, e.g., inducing inhibition, regression, or stasis of the disorder and/or disease. As used herein, “inhibition” of disease progression or disease complication in a subject means preventing or reducing the disease progression and/or disease complication in the subject.

As used herein, a subject who has experienced “thiopurine failure” has been treated with a thiopurine, including, for example, 6-mercaptopurine or azathioprine, and either suffered an adverse side effect or did not experience a clinical response or clinical benefit.

As used herein, “remission” in a subject or patient suffering from Crohn's disease refers to when their CDAI score is <150. “Remission” in a subject or patient suffering from Ulcerative Colitis refers to when their total Mayo score is 0, when their total Mayo score is less than or equal to 2, or when their total Mayo score is less than or equal to 2 with no category score above 1.

As used herein, a “response” refers to when a subject or patient's CDAI score decreases by 100 points or more from baseline.

As used herein, a “clinical response” or “clinical benefit” refers to when a subject's CDAI score decreases by 100 points or more from baseline, or when the subject's CDAI score is <150 (i.e. either a response or a remission). A patient who is “unresponsive” did not experience a clinical response.

As used herein, “maximal clinical response” refers to the point when a patient's CDAI score is at or about at the lowest it will be during the course of a subject's treatment.

As used herein, “mucosal healing” refers to any improvement in the inflamed intestinal tissue implicated in Crohn's disease. As non-limiting examples, this may include reduction in or elimination of inflammation, ulcerations, and erosions of the tissue.

As used herein, a patient who is “administered steroids” refers to a patient who is administered one or more types of steroid. A patient who is “administered antibiotics” refers to a patient who is administered one or more types of antibiotic.

As used herein, a subject who “did not experience a clinical response to previous thiopurine administration” refers to a subject who was previously administered a thiopurine, but did not experience a clinical response to that administration.

As used herein, “steroid-dependent” refers to, for example, subjects with active Crohn's disease, with CDAI score between 220-450 at screening, in spite of constant steroid treatment.

As used herein, “Intent to Treat” (ITT) population included all randomized/enrolled patients who received a subject study number, signed the informed consent form (ICF) and received at least one dose of study medication. The ITT population included 64 subjects (40 subjects in the DR-6MP 80 mg treatment arm and 24 subjects in the Purinethol treatment arm).

As used herein, “Safety population” is the ITT population.

As used herein, “Per Protocol” (PP) Population included all randomized subjects who completed the study according to the protocol. The PP population included 37 subjects (25 subjects in the DR-6MP 80 mg treatment arm and 12 subjects in the Purinethol treatment arm).

As used herein, “Modified Intent To Treat” (mITT) Population included all PP subjects as well as those patients who dropped out at/after week 6 (visit 7) and at/after week 8 (visit 8) with Last Observation Carried Forward (LOCF) as their final observation. The populations were named mITT1 (Week 6) and mITT2 (Week 8).

### Experimental Details and Discussion

### Investigational Plan

### Overall Study Design and Plan: Description

This was a Phase IIa, multi-center, randomized, double-blind, double-dummy, parallel group, two arm, 12 week study to evaluate the clinical efficacy and safety of 80 mg. oral delayed release 6-mercaptopurine (DR-6MP) versus PURINETHOL® (at the standard dose of 1-1.5 mg/kg/day) for 12 weeks in subjects with moderately active (CDAI 220-450, inclusive) Crohn's disease.

One-hundred and one (101) subjects were screened within 1-2 weeks prior to baseline and 70 eligible subjects were enrolled and randomized in a 2:1 randomization scheme to either DR-6MP or PURINETHOL®:

Test: DR-6MP 80 mg administered orally as 2×40 mg delayed release 6-MP tablet, at bedtime, Q.D. (once daily).

Reference: PURINETHOL® 1-1.5 mg/kg (DSM, Gates Pharmaceuticals) administered orally, as a tablet (50 mg) in the morning hours, Q.D. (once daily). All subjects randomized to this treatment will take 3 tablets; however, depending on the dose, the number of active tablets can range from 1-3 active tablets per day.

Since (a) the DR-6MP tablet and PURINETHOL® tablet are available only in single units of 40 mg and 50 mg, respectively, whereas the doses of each to be administered are higher (i.e., 80 mg or 50-150 mg, respectively) and (b) the DR-6MP tablet and PURINETHOL® tablet are not similar in appearance, and (c) several different doses for PURINETHOL® can be administered, a double-blind, double-dummy blinding scheme was necessary. Therefore, in order to provide all possible study doses and still maintain the blind, all subjects, regardless of treatment allocation, were required to take two (2) DR-6MP tablets (active or placebo) once nightly, and three (3) PURINETHOL® tablets (active or placebo) in the morning hours, once daily.

The possible study drug assignments to allow for each treatment arm and for each dose permutation within the PURINETHOL® arm are tabulated below, in which each row depicts a daily dose, and “A” is active and “P” is placebo:

TABLE 1
Study dosing scheme

| | | DR- | DR- | | | |
|---|---|---|---|---|---|---|
| | Dose | 6MP | 6MP | PURINETHOL ® | PURINETHOL ® | PURINETHOL ® |
| Treatment Arm | (mg) | Tablet | Tablet | Tablet | Tablet | Tablet |
| 80 mg DR-6MP | 80 | A | A | P | P | P |
| 1-1.5 mg/kg/day | 50 | P | P | A | P | P |
| PURINETHOL ® | 75* | P | P | A | P | P |
| | | P | P | A | A | P |
| | 100 | P | P | A | A | P |
| | 125* | P | P | A | A | P |
| | | P | P | A | A | A |
| | 150 | P | P | A | A | A |

*The two rows depicted represent two days so that the average dose for the week is the dose noted

All subjects were required to complete a daily dosing diary.

Scheduled in-clinic visits were conducted at screening, baseline, week 1, week 2, and every two weeks up to week 8, and at the final visit (week 12). Unscheduled visits for safety or for any other reason were conducted at any time during the study.

During the study period, CDAI score was assessed at screening, baseline and every visit (except week 1). Subjects were required to complete a daily CDAI questionnaire every morning on each of the seven days before a scheduled clinic visit at which the CDAI was assessed.

Systemic immunological improvement was evaluated during the study period: General systemic immunological markers CRP and ESR were evaluated at baseline and at weeks 2, 4, 6, 8 and 12. IFN-γ (on a subset of subjects) and immunological markers (FACS analysis: all subjects) on peripheral lymphocytes for surface marker expression levels (of CD4; CD8; CD4, CD25, FOXP3; CD3, CD56; and CD4, CD62, CD127) were evaluated at baseline and at week 12.

Colonoscopy and ileoscopy (at all sites on a subset of subjects who agreed to the procedure) with evaluation of intestinal tissue by CDEIS were performed once during the two week period of screening/baseline and again at week 12.

The previous standard of care treatment allowed at study entry and throughout the study was 5-aminosalicylic acid (5-ASA) at a stable dose (≧2 weeks prior to screening). Additionally, subjects considered by the Principal Investigator (PI) to be steroid-dependent or antibiotic dependent (i.e., subjects with active Crohn's disease, with CDAI score between 220-450 at screening, in spite of constant steroid or antibiotic treatment), were allowed to enroll in the study, on low-dose oral steroids or antibiotics, provided that they were on a stable dose (≧2 weeks prior to screening), and remained on that dose throughout the study. For oral steroids, low-dose is <15 mg prednisolone daily or <6 mg budesonide daily.

Furthermore, steroids rescue therapy was allowed during the study if symptomatic relief was required, as determined by the PI. Oral prednisolone, at a starting dose of 40-60 mg/day with a variable tapering dose, was allowed starting from week 2 until week 6, to achieve steroid-free remission at week 12.

### Discussion of the Study Population

### Selection of the Study Population

Subjects who met the eligibility criteria were randomized in 2:1 randomization scheme to one of the following treatment arms: 80 mg DR-6MP (Test) or 1-1.5 mg/kg/daily PURINETHOL® (Reference).

A total of 70 subjects were randomized, with 46 subjects randomized to the DR-6MP treatment group, and 24 to the PURINETHOL® arm.

### Inclusion Criteria

Subjects had to meet all the following inclusion criteria to be eligible:
1. Male and (non-pregnant) female subjects, aged 18-75 years (inclusive) at screening.
2. Subjects with a diagnosis of CD appropriately documented and supported by endoscopy or radiology.
3. Subjects with current moderately active CD with screening CDAI score of 220-450 (inclusive).
4. Screening laboratory tests had to meet the following criteria:
   Hemoglobin (HGB)≧8.5 g/dL
   Platelets≧100,000/mm³
   WBC≧3500 mm³
   Serum albumin>2.5 g/dL
   Alanine aminotransferase (ALT), (aspartate aminotransferase) AST, alkaline phosphatase (ALKP), gamma glutamyl transpeptidase (GGTP), total and direct bilirubin <2× upper limit of normal (ULN)
5. Subjects may be on stable (for at least 2 weeks prior to screening) 5-aminosalicylic acid (5-ASA), chronic antibiotics or low-dose oral steroids (prednisolone—up to 15 mg daily; budesonide—up to 6 mg daily), and remain on the drug at that dose throughout the study.
6. Subjects willing and able to provide written informed consent.

### Exclusion Criteria

Any of the following conditions at screening excluded the subject from entering the study:
1. Subjects with ulcerative colitis or with a diagnosis of indeterminate colitis.
2. Subjects with previous bowel resection due to CD resulting in clinically significant Short Bowel Syndrome.
3. Subjects with fistulizing CD with clinical or radiological evidence of abscess.
4. Subjects with clinically significant GI obstructive symptoms (i.e., symptomatic stenosis or ileal strictures) or x-ray evidence of fibrosed bowel.
5. Subjects with positive results on screening stool culture for enteric pathogens (Salmonella, Shigella, Campylobacter) and Clostridium difficile toxin assay.
6. Subjects with history of persistent intestinal obstruction, bowel perforation, uncontrolled GI bleed or abdominal abscess or infection or toxic megacolon.
7. Subjects with a history of GI tract malignancy or IBD-associated malignant changes in the intestines.
8. Subjects with surgery/major procedure in the 4 weeks prior to the first study dosing.
9. Subjects receiving an elemental diet or parenteral nutrition.
10. Subjects with current signs or symptoms of a clinically significant or unstable medical or surgical condition that, in the Investigator's opinion, would preclude safe and complete study participation, as determined by medical history, physical examination, ECG, laboratory tests or imaging. Such conditions may include severe, progressive or uncontrolled renal, metabolic, hepatic, hematological, endocrine, pulmonary, cardiovascular, psychiatric, neurological, cerebral or autoimmune disease.
11. Subjects with serious infections, such as hepatitis, pneumonia, pyelonephritis within 12 weeks prior to the first study dosing. Less serious infections within 12 weeks prior to the first study dosing, such as acute upper respiratory tract infections (colds) or uncomplicated urinary tract infection need not be considered exclusions—at the discretion of the Principal Investigator.
12. Subjects with any currently known malignancy or pre-malignant lesions or any history of malignancy within the past 5 years, excluding basal cell carcinoma.
13. Subjects with a history of coagulopathy.
14. Subjects with porphyria as it may interfere with assessment of CD abdominal pain.
15. Subjects with history of previous thiopurine failure resulting in a serious adverse reaction (such as, severe pancreatitis, severe leucopenia, severe hepatotoxicity or bone marrow suppression) so as to preclude additional treatment with 6-mercaptopurine at any dose.
16. Subjects who have received within 6 months prior to first study dosing (and at any time during the study):
   Active vaccinations, i.e., live, attenuated bacterial/viral pathogens
17. Subjects who have received within 6 weeks prior to first study dosing (and throughout the study):
   Anti-tumor necrosis factor α (TNF-α) (infliximab, etanercept, adalimumab)
   Anti-integrin (natalizumab)
   Anti-neoplastics, including methotrexate, daunorubicin hydrochloride
18. Subjects who have received within 4 weeks prior to first study dosing (and throughout the study):
   Immunosuppressants such as azathioprine, 6-MP (i.e., other than 6MP study drug assigned during study), cyclosporine, tacrolimus, mycophenolate mofetil or thalidomide.
   Treatment with any drugs known to induce or inhibit endogenous hepatic drug metabolism such as barbiturates, phenothiazines, cimetidine, carbamazepine etc.
   Anti-coagulant therapy such as: heparin, warfarin, acenocoumarol.
   Medications that induce blood dyscrasias or have potential for immune dysfunction, bone marrow depression and/or symptoms of CD (diarrhea, abdominal pain).
   Vaccinations involving inactivated forms of pathogens or purified antigenic proteins.
Note: Passive immunization involving antibody inoculations were allowed at any time.
19. Subjects who took within 2 weeks prior to first study dosing (and throughout the study):
   IV or oral steroids (prednisolone or budesonide)
   Antibiotics
      Note: There were two important exceptions to this criterion: (a) Subjects who were oral steroid or antibiotic-dependent and had active CD (CDAI score of 220-450) in spite of being on these treatments, could remain on these therapies provided they were on a stable (≧2 weeks at screening) dose and remained on that dose throughout the study (See Inclusion Criterion #5); (b) Subjects who required “steroid-rescue” therapy during the study.
20. Subjects who took within 7 days prior to first study dosing (and throughout the study):
   Anticholinergic or other drugs known to affect gastrointestinal motility
   Proton-pump inhibitors or other drugs known to affect gastric acidity
   Allopurinol
21. Subjects with a body weight below 42.5 kilograms.
22. Women who were pregnant or nursing at the time of screening, or who intended to be pregnant or nursing during the study period.
23. Women of childbearing potential who were not practicing an acceptable method of birth control [acceptable methods of birth control are: surgical sterilization, intrauterine devices, oral contraceptive, contraceptive patch, long-acting injectable contraceptive, partner's vasectomy, a double-protection method (condom or diaphragm with spermicide) or abstinence].
24. Subjects with current or history of drug and/or alcohol abuse.
25. Subjects who were largely or wholly bed-ridden and who had little capacity for self-care.
26. Subjects with known allergy or hypersensitivity to 6-MP or any inactive component of the study drug (e.g. subjects who are lactose intolerant).
27. Subjects who participated in another clinical trial using investigational drugs within 12 weeks prior to the first study dosing.
28. Subjects with planned elective surgery or hospitalization during the course of the study (that may interfere with study compliance or outcome).
29. Subjects with an inability to communicate well with the investigators and staff (i.e., language problem, poor mental development or impaired cerebral function).
30. Subjects who were unavailable for the duration of the trial, were unable to comply with the planned schedule of study visits, were likely to be noncompliant with the protocol, or who were felt to be unsuitable by the investigator for any other reason.
   Removal of Subjects from Therapy or Assessment

Each subject was informed of his/her right to withdraw from the study at any time and for any reason. The investigator could withdraw a subject from the study at any time if he/she considered that remaining in the study compromised the subject's health or that the subject was not sufficiently cooperative. The reasons for any subject withdrawal were recorded on the study completion form of the CRF.

Pregnant or nursing subjects had to be withdrawn from the trial.

When a subject withdrew from the study, all of the safety data, normally required at the end of the study, were obtained if possible. Dropouts were not replaced.

### Treatments

### Method of Assigning Subjects to Treatment Groups

Following determination of eligibility, a “Randomization Eligibility Form” was completed by the site PI or his designee for each eligible subject and included the following information: 1) Screening Number/Initials; 2) Gender; 3) Date of birth; 4) Weight; 5) Newly diagnosed CD (Yes/No); 6) Screening/Randomization CDAI score; 7) Anticipated baseline visit date; 8) Previous thiopurine failure either due to lack of clinical benefit or occurrence of non-serious adverse events (Yes/No).

Elements 2-5 were intended to ensure that there is a balanced representation in both treatment arms; however, due to the limitations of the randomization algorithm, only gender, age and weight were used. Element 8 was considered in order to ensure subject safety; such subjects were randomized only to the DR-6MP treatment arm.

### Selection and Timine of Dose for Each Subiect

All study drugs were administered with 240 ml water at room temperature.

DR-6MP 80 me

Subjects randomized to DR-6MP at baseline received 80 mg and remained at that dose for 12 weeks. Since the DR-6MP tablet is available only as a single 40 mg tablet, the DR-6MP treatment arm was provided as two tablets.

PURINETHOL®

Subjects randomized to PURINETHOL® treatment followed the dosing paradigm below:

1. Initial dose: 1.0 mg/kg (body weight at screening). The dose was set by Medistat, based on the weight information completed by the site for the subject on the “Randomization Form”, and according to the table below:

TABLE 2
Determination of Initial PURINETHOL ® dose

| Initial | Subject Screening Weight |
|---|---|
| PURINETHOL ® Dose | as completed on |
| (1 mg/kg/day) | Randomization Sheet (kg) |
| 50 mg | 42.5-62 |
| 75 mg | 63-87 |
| 100 mg | 88-112 |
| 125 mg | 113-137 |
| 150 mg | 138-162 |

The Initial dose was generally maintained for a period of 4 weeks.

2. Titration Dose: Based on the lab data at week one and week 2, the Study Safety Physician could have increased the subject's PURINETHOL® dose to 1.5 mg/kg body weight at Week 4 according to Table 3 below.

TABLE 3
Determination of PURINETHOL ® titration dose

| Initial | Increased |
|---|---|
| PURINETHOL ® Dose | PURINETHOL ® |
| (1.0 mg/kg) | Dose (1.5 mg/kg) |
| 50 mg | 75 or 100 mg |
| 75 mg | 100 or 125 mg |
| 100 mg | 125 or 150 mg |
| 125 mg | 150 mg |
| 150 mg | 150 mg |

Alternatively, the Study Safety Physician could have decided, on the basis of the subject's laboratory test results, not to increase the dose, and to maintain the subject at 1.0 mg/kg body weight for a longer period of time. This decision was reviewed at each visit by the Study Safety Physician following each subject's laboratory test results.

3. Change in dose following titration: the Study Safety Physician could decrease the PURINETHOL® dose to 1.0 mg/kg, if a subject at the 1.5 mg/kg dose, had laboratory test results that indicated that the higher dose was not being tolerated. Once the decrease was made, the subject dose could not be increased back up again to the maximum 1.5 mg/kg during the remainder of the study.

To maintain blinding, all subjects, regardless of treatment assignment, were required to take 5 tablets daily: two DR-6MP (active or placebo) tablets at night, prior to bedtime (subject will remain in an upright position for 10 minutes following dose ingestion) and three PURINETHOL® (active or placebo) tablets in the morning hours. For those randomized to the DR-6MP arm, the DR-6MP was active while the “PURINETHOL®” was a placebo. Similarly, for those randomized to the PURINETHOL® arm, the PURINETHOL® was active, while the “DR-6MP” was a placebo.

The times of drug administration above correspond to standard clinical practice, in which most subjects take PURINETHOL® during the morning hours, whereas the night-time dosing of DR-6MP is based on the previous clinical study, in which efficacy was demonstrated following night-time dosing. Moreover, dividing the doses into night-time and morning administrations was done to enhance subject convenience and compliance. However, if the subjects experienced nausea or other discomfort when taking the morning tablets, the PI could advise the subject to take all 5 tablets at night, prior to bedtime. All dose administrations (dates and times) were documented on the subject dosing diary on each Dose Card. From Week 1 and onward, the Study Safety Physician reviewed the unblinded laboratory test results for each subject and made an appropriate treatment determination within 24 hours of receipt of the results from the central laboratory (AML), as displayed in the table below:

TABLE 4
Dose modifications

| Appropriate | | |
|---|---|---|
| Treatment | | |
| Determination | Relevant for: | Change to |
| Continue on | Subjects in both | NA |
| current dose | treatment arms | |
| Increase Dose | Subjects currently on | PURINETHOL ® |
| | PURINETHOL ® at | at 1.5 mg/kg/day |
| | dose of 1 mg/kg/day | |
| Decrease Dose | Subjects currently on | PURINETHOL ® |
| | PURINETHOL ® at | at 1 mg/kg/day |
| | dose of 1.5 mg/kg/day | |
| Permanently | Subjects currently on | Subject terminated |
| Discontinue | PURINETHOL ®, | from study |
| | exhibiting severe | |
| | leucopenia or alterations | |
| | in liver function tests | |
| | (see section on Red | |
| | Alerts, as defined below) | |
| Temporarily or | Subjects in both | As per Study Safety |
| Permanently | treatment arms | Physician discretion on a |
| Discontinue | | case-by-case basis |

The Study Safety Physician's decision as to the appropriate treatment was based on the following safety considerations:

Laboratory test parameters upon enrollment
The Study Safety Physician received from AML laboratories the data of the screening and baseline laboratory test results for all enrolled subjects and set up a database of subject data to monitor the subject's status relating to the important laboratory test parameters even before the subject has started drug treatment.

Drug Dose to be Decreased
The appearance of any of the following laboratory test results for a subject at any visit following baseline, resulted in the Study Safety Physician's appropriate treatment determination to decrease drug dose:
   WBC<3000 mm³
   ALT>2×ULN (upper limit of normal)
   AST>2×ULN (upper limit of normal)
   Total bilirubin>2×ULN (upper limit of normal)
   Direct bilirubin>2×ULN (upper limit of normal)
This appropriate treatment determination applied to subjects on PURINETHOL®. As no further decrease in dose was possible for subjects on 80 mg DR-6MP, a temporary discontinuation of drug for either treatment arm was also considered at the Study Safety Physician's discretion.
   “Red Alert”: stopping of drug treatment
The appearance of any of the following laboratory test results for a subject at any visit following baseline, resulted in immediate stopping of drug treatment by the Study Safety Physician:
   ANC (absolute neutrophil count)<1000 mm3
   ALT≧5×ULN (upper limit of normal)
   AST≧5×ULN (upper limit of normal)

### Prior and Concomitant Therapy

Medications Allowed at Study Entry and During the Study
Stable dose (≧2 weeks prior to screening) of 5-ASA compounds was allowed at study entry and during the study. Adjunctive use of 5-ASA was allowed in all previous studies conducted with the DR-6MP test tablet (pilot, proof-of-concept clinical efficacy and PK studies). Although not indicated for CD treatment, 5-ASA compounds are typically used to treat active CD in clinical practice. Due to their limited efficacy, it was deemed unlikely that 5-ASA would interfere with the efficacy assessment in the study, but that this therapy may provide some relief while the subjects were awaiting determination of screening eligibility.
Stable dose of low-dose steroids or antibiotics were allowed at study entry and during the study. Subjects considered by the PI to be steroid-dependent or antibiotic dependent were allowed to enroll in the study on low-dose oral steroids or antibiotics, provided that they were on a stable dose (≦2 weeks prior to screening), and remained on that dose throughout the study. For oral steroids, low-dose was ≦15 mg prednisolone daily or ≦6 mg budesonide daily. The fact that these subjects entered the study with active CD, with CDAI score between 220-450, in spite of constant steroid or antibiotic treatment indicates that in these subjects, these treatments have not been agents of remission induction. Rather, the clinical efficacy to induce remission or clinical response in these subjects was assessed following the addition of either treatment arm (DR-6MP or PURINETHOL®) as add-on therapy.

Additional Treatment Regimens Allowed to be Initiated During the Study
Steroids rescue therapy: rescue therapy was allowed during the study for those subjects requiring symptomatic relief after at least 2 weeks on the study. Steroids rescue treatment (40-60 mg/day starting dose) could be initiated in the period from week 2 and up until week 6, so that following the steroid tapering regimen, all subjects requiring steroids rescue had to be steroid-free at the week 12 final visit.
Treatment for symptomatic relief of diarrhea (e.g. use of diphenoxulate, loperamide, or other opioids for diarrhea) was allowed at study entry and during the study.

### Disallowed Concomitant Medications

See Exclusion Criteria, above.

If any of the disallowed drugs were used, the subject could be dropped from the study.
Vaccinations—Due to increased risk of infection with live vaccine to immunocompromised subjects, no such vaccinations were allowed for six months prior to first study dosing and throughout the study. Vaccinations involving immunization with killed or inactivated pathogenic forms were not allowed for 4 weeks prior to first study dosing, and throughout the study. There was no wash-out period for passive immunization involving antibody inoculations; this type of vaccination was allowed before and at any time during the study.

Treatment Compliance

Subjects were given supplies at each of the clinical evaluation visits. All study drugs had to be accounted for during the study. Drug dispensing and returns of the medication were documented by the site study staff on the individual case report forms and by the monitor's Drug Accountability Records. Additionally, throughout the treatment period, subjects were required to maintain a daily dosage diary card to verify ingestion of dose.

Subjects were also required to bring their empty Dose Cards as well as unused tablets back with them to the clinic. Compliance with the study regimen was checked by counting returned tablets and checking the subjects' diaries.

### Efficacy and Safety Variables

### CDAI Score

The CDAI score was measured for all subjects at all sites during the run-in period (screening), baseline, and all subsequent visits, except for the week 1 visit.

The CDAI Score was the main clinical assessment used for determining drug efficacy [Best et al. 1976; Sandbom et al. 2002]; it provides a standardized index for measuring disease activity and is the most widely used instrument in clinical trials of CD. It is a validated, weighted index based on signs and symptoms of CD, physical examination and hematocrit measurement. It is comprised of 8 variables: the number of liquid/soft stools per day, the extent of abdominal pain (none, mild, moderate, severe), general well-being (generally well, slightly under par, poor, very poor, terrible), the need for anti-diarrheal drugs (use of diphenoxulate, loperamide, or other opioids for diarrhea), the occurrence of extra intestinal symptoms during past week (complications including arthritis, iritis, fever, etc.), the presence of abdominal masses (absent, questionable, definite), hematocrit, and body weight. The first 4 of these variables and the presence of fever above 37.8° C. were self-reported in subject diaries; the remaining 4 were assessed at the study visit. Height and standard weight assessment were based on standard height-weight tables.

Total CDAI scores range from 0 to approximately 600 where the higher the score, the more active the disease. A CDAI score of less than 150 points denotes remission; between 150 to 219 points denotes mildly active disease; between 220 to 450 points denotes moderately active disease; and more than 450 points denotes severe disease. Remission is defined as reduction in CDAI score to a total score below 150 points and response is defined as either remission, or as reduction of at least 100 points in the total CDAI score compared to baseline, at the end of the treatment period [EMEA 2007].

Subjects were required to maintain daily CDAI diary forms during the entire study period (screening period [up to 2 weeks] and treatment period [12 weeks]). The scores obtained from the seven daily consecutive diaries completed prior to the baseline visit and to each of weeks 2, 4, 6, 8 and 12 contributed to the total CDAI score at each of these time points.

### Inflammatory Bowel Disease Questionnaire (IBDQ)

The IBDQ evaluates the patient's quality of life using elements of social, systemic and emotional symptoms, as well as bowel related symptoms. The questionnaire contains 32 questions evaluating general activities of daily living, intestinal function such as bowel habit and abdominal pain, as well as social performance, personal interactions, and emotional status. Responses are graded on a seven point Likert scale, from 7 (not a problem at all) to 1 (a very severe problem). A higher score indicates better quality of life. Responses are also grouped into four categories as Bowel (10 items), Systemic (5 items), Social (5 items), and Emotional (12 items) dimensions [Guyatt et al. 1989].

### CDEIS Score (Colonoscopy/Ileoscopy)

Colonoscopy/ileoscopy was conducted on a subset of subjects who agreed to undergo the procedure, at two time points: at pre-treatment (during the screening/baseline period) and at week 12.

The Crohn's Disease Endoscopic Index of Severity (“CDEIS”) is a commonly-accepted clinical measure of mucosal healing in CD. It is based upon the presence or absence of 4 types of lesions: superficial ulcers, deep ulcers, ulcerated stenosis, or non-ulcerated stenosis, all of which are recorded in 5 different segments: terminal ileum, ascending colon, transverse colon, descending and sigmoid colon, and the rectum. In addition, for each of these 5 segments, the surface area of diseased and/or ulcerated individual segments were measured in cm (generally reported from 1-10 cm), with a total in cm provided for the diseased and/or ulcerated surface areas. The combination of values allows calculation of the score severity [Sostegni et al. 2003].

### Immunology

Immunology tests were conducted for all subjects at baseline and all subsequent visits (except visit week 1) for the general inflammatory markers: CRP and ESR.

Study eligibility in terms of the CDAI score was based upon screening visit CDAI. Subjects who failed to meet the CDAI inclusion criteria based on their baseline hematocrit results or other CDAI parameters were not withdrawn from the study or replaced. Nevertheless, the baseline CDAI was used as the pre-treatment reference value when conducting the study analyses.

The evaluation of peripheral lymphocytes was conducted by FACS analysis at baseline and visit Week 12 for all subjects. The evaluation of IFN-γ in peripheral lymphocytes was conducted only in the subset of subjects undergoing colonoscopy/ileoscopy procedures at baseline and Week 12. In addition to the blood samples required for the IFN-γ assay, 4 biopsy samples taken during the colonoscopy/ileoscopy procedure were needed for the assay for subject-specific antigen determination.

### CRP

C-reactive protein (CRP) is an inflammatory mediator whose levels are raised under conditions of acute inflammatory recurrence and rapidly normalize once the inflammation subsides. CRP may serve as a surrogate marker to monitor inflammatory disease activity and response to treatment.

CD may be characterized according to disease behavior: predominantly non-stricturing, non-penetrating (inflammatory), stricturing or penetrating. Predominantly non-stricturing, non-penetrating (inflammatory) CD may be characterized by high CRP levels.

The median concentration of CRP in moderate to severe CD subjects in previous pivotal trials reported in the literature was 9-12 mg/L while the mean concentrations reached a value of 20-23 mg/L [Colombel et al. 2007; Sandborn et al. 2007].

The Erythrocyte Sedimentation Rate (ESR) is the rate at which red blood cells precipitate in a period of 1 hour. It is a common hematology test which is a non-specific measure of inflammation. ESR is used to measure the acute phase response to screen for the presence of infection or inflammation and to monitor disease activity [Vermeire et al. 2006].

### Interferon Gamma (IFN-γ)

In CD, the aberrant immune response is regulated by Type 1 T helper cells, resulting from the proliferation and differentiation of T cells into effector T cells, producing cytokines that magnify the immune response. Such cytokines may include IFN-γ, interleukin 2 (II2), and IL-18. Due to defective apoptosis, the reaction of the immune response does not terminate, and results in an ongoing exaggerated T-cell response. A broad cascade of inflammatory mediators such as TNF-α are quantifiable.

Hence, another index of clinical improvement in CD is modification of the circulating blood levels of systemic immunologic cells, including serum and intracellular cytokines, T cell subsets and specifically IFN-γ [Fuss et al. 1996]. Whereas ulcerative colitis intestinal LP cells manifest increased secretion of IL-5, CD LP cells manifest increased secretion of IFN-γ. Hence, as a surrogate marker monitoring immunologic response, a reduction in IFN-γ levels, as measured using the highly sensitive enzyme-linked immunosorbent spot (ELISPOT), indicates improvement in the CD patient's immunological status. The test measures the number of T cell clones secreting IFN-γ in response to the patient-derived bowel proteins.

FACS Analysis of Lymphocytes Isolated from Peripheral Blood

Tregs play an important role in the pathogenesis of CD. Tregs actively suppress enteroantigen-reactive cells and contribute to the maintenance of intestinal immune homeostasis. Distinct Treg subsets coexist in the intestinal mucosa and have been shown to be important to prevent and/or cure colitis. Failure to control these responses disrupts tolerance, and this is proposed to be one of the mechanisms involved in the development of inflammatory bowel disease [Roncarolo et al. 2007; Shevach et al. 2006; Ochi et al. 2006; Schurmann et al. 1995].

Modification of the circulating blood levels of systemic immunologic cells, including serum and intracellular cytokines, T cell subsets and specifically IFN-γ provide an index of clinical improvement in CD.

There is recent evidence from both animal and human studies to indicate that oral administration of low dosage administration of immunomodulatory agents is an effective means for activation of these regulatory T cells [Ilan et al. PNAS 2010; Ilan et al. JCI 2010; da Cunha et al. 2012; Wu et al. 2009].

In order to determine the immunological effect of the DR-6MP, as compared to PURINETHOL®, FACS analysis was performed on peripheral blood lymphocytes collected at baseline and week 12 for all subjects. Lymphocytes were tested at the Hadassah Medical Center Immunology Laboratory for surface marker expression including levels of:
CD4
CD8
CD4, CD25
CD4, CD25, FOXP3
CD3, CD56
CD4, CD62, CD127

### Safety Measurements

### Adverse Events (AEs)

All adverse experiences, whether observed by the Principal Investigator or his designee, elicited from the subject, or volunteered by the subject, commencing with signature of ICF, were recorded in the subject's CRF, regardless of whether or not they was considered to be related to study medication. AEs were reviewed and updated at each subsequent visit and during any phone contact with the subject.

### AEs Causality

The following definitions were used by the investigating physician to describe the relationship between an AE and the study drug (test or reference).

No Reasonable Possibility:

This category applies to those AEs which, after careful consideration, were clearly due to extraneous causes (disease, environment, etc.) or to those AEs, which after careful medical consideration at the time they were evaluated, were judged to be unrelated to the study drug (test or reference).

Clarification: An adverse experience may have been considered “No Reasonable Possibility” if it was clearly due to extraneous causes or when (at least two of the following):
It did not follow a reasonable temporal sequence from the administration of the study drug.
It could readily have been produced by the subject's clinical state, environmental or toxic factors, or other modes of therapy administered to the subject.
It did not follow a known pattern of response to the study drug.
It did not reappear or worsen when the study drug was re-administered.

Reasonable Possibility:

This category applies to those AEs for which, after careful medical consideration at the time they were evaluated, a connection with the study drug (test or reference) could not be ruled out with certainty or felt with a high degree of certainty to be related to the study drug.

Clarification: An adverse experience may have been considered “Reasonable Possibility” related if or when (at least two of the following):
It followed a reasonable temporal sequence from administration of the study drug.
It could not be reasonably explained by the known characteristics of the subject's clinical state, environmental or toxic factors or other modes of therapy administered to the subject.
It disappeared or decreased on cessation or reduction in dose. There are important exceptions when an AE does not disappear upon discontinuation of the study drug, yet drug-relatedness clearly exists.
It followed a known pattern of response to the study drug.

### Safety Laboratory Evaluation

All laboratory testing were performed by AML, a central laboratory facility selected by the Sponsor. Laboratory tests were performed at each scheduled study visit (unless otherwise specified) and at an unscheduled visit, upon need.

The following laboratory tests were performed:
Serum chemistry: Glucose, Sodium, Phosphorus, Potassium, Urea, Creatinine, AST, ALT, GGTP, Lactate dehydrogenase (LDH), Albumin, Total protein, Calcium, Alkaline Phosphatase, Amylase, Total bilirubin, Direct bilirubin, Creatinine phosphokinase (CPK)
Hematology: Red Blood Cell Count (CBC), Hemoglobin (Hgb), Hematocrit (Hct), Mean Cell Hemoglobin (MCH), Mean Cell Hemoglobin Concentration (MCHC), Mean Corpuscular Volume (MCV), Red Cell Distribution Width (RDW), White Blood Cell (WBC) Count and Differential, Platelets
Coagulation panel (Screening visit only): Prothrombin time (PT), Activated partial thromboplastin time (aPTT), International normalized ratio (INR)
General Immunology (all visits, except Screening and Week 1): CRP, ESR
Urinalysis: Protein, Glucose, Specific Gravity, Ketones, Urobilinogen, Bilirubin, pH, Erythrocytes, Leukocytes, Nitrites
Stool culture (Screening visit only): Stool culture for enteric pathogens (Salmonella, Shigella, Campylobacter) and Clostridium Difficile toxin assay.
Pregnancy test (Screening, Baseline and Week 12): Serum 13-human chorionic gonadotropin (HCG) in females of childbearing potential.

### Vital Signs

Vital signs (temperature, pulse and systolic and diastolic blood pressure) and weight were recorded at all study visits. Blood pressure and pulse rate were measured after the subject had rested comfortably for five minutes. Blood pressure was to be taken always from the same arm, routinely the right arm.

### Electrocardiogram (ECG)

ECGs were performed at screening, baseline and week 12. The 12-lead ECG was evaluated by the Investigator or a qualified designee at time of performance (signed and dated) and the printout was kept in the source documentation file.

### Physical Examination

A comprehensive physical examination was performed and documented by the Investigator or a qualified designee, at all study visits.

### 6MP Drug Safety

All laboratory results were continuously reviewed by the 6MP Drug Safety Physician, with particular attention paid to WBC, ANC, ALT, AST, direct and total bilirubin levels.

### Appropriateness of Measurements

### CDAI Score

The CDAI Score was the main clinical assessment used for determining drug efficacy [Best et al. 1976; Sandbom et al. 2002]; it provides a standardized index for measuring disease activity and is the most widely used instrument in clinical trials of CD. It is a validated, weighted index based on signs and symptoms of CD, physical examination and hematocrit measurement.

### CDEIS

CDEIS is a commonly-accepted clinical measure of mucosal healing in CD. The severity of mucosal inflammation, as assessed by colonoscopy/ileoscopy, has been touted an additional mainstay parameter for efficacy assessment in clinical trials. Moreover, the necessity for treatment to induce mucosal tissue healing in inflammatory bowel disease, in general, has become clinically relevant in light of recent reports correlating disease activity with a patient's overall risk of developing colorectal cancer. Therefore, the assessment of mucosal healing in a subset of patients willing to undergo colonoscopy/ileoscopy has been included as one of the secondary efficacy parameters in this study. This efficacy benchmark was clearly evident in the previous pilot feasibility study, in the CDEIS scores and colonoscopy narrative reports for DR-6MP subjects.

### Immunological Biomarkers

Modification of the circulating blood levels of systemic immunologic cells, including serum and intracellular cytokines, T cell subsets and specifically IFN-γ provide an index of clinical improvement in CD.

### Safety Assessments

The safety parameters selected for the study are standard for this indication/patient population. In addition to the standard AEs and SAEs, we looked at CD-specific hepatotoxicity, leucopenia, pancreatitis and their relevant labs, as well as weight change.

### Primary Efficacy Variable

The primary efficacy variable was clinical response at week 12. Selection of 12 weeks as the primary endpoint time point was a departure from standard study design, done specifically at the request of the study principal investigators to address the expected treatment response time for PURINETHOL®.

The proportion of patients achieving clinical remission (i.e., CDAI<150 maintained for 2 weeks) within the period of about 4 to 8 weeks, based on the pharmacodynamics properties of the test drug, is an appropriate primary endpoint to justify short-term treatment of active CD. Nevertheless, a 12 week treatment period was selected for this study as standard 6MP (PURINETHOL®) typically has a slow onset of action, and requires about 3-4 months of administration before its therapeutic effects in CD become apparent. The study was thus suitably designed to evaluate both the difference in therapeutic gain (remission/clinical response) between treatments, as well as the time to achieve therapeutic gain for each treatment arm.

### Study Subjects

### Disposition of Subjects

Subject disposition is shown in Table 6. Seventy subjects were enrolled in this study and randomized. Forty-six subjects were randomized into the DR-6MP treatment arm and 24 into the PURINETHOL® treatment arm. 26 (56.5%) and 13 (54.2%) subjects from the DR-6MP and PURINETHOL® treatment arms, respectively, completed the study. 6 subjects from the DR-6MP treatment arm were excluded from the study—2 subjects (4.3%) were excluded because they received a DR-6MP dose other than 80 mg. 4 subjects (8.7%) were excluded because they never started the treatment.

TABLE 5
Disposition of subjects

| | DR-6MP 80 mg | | PURINETHOL ® | |
|---|---|---|---|---|
| | N = 46 | | N = 24 | |
| Study Disposition | N (%) | | N (%) | |
| Subject completed the study | 26 | (56.5) | 13 | (54.2) |
| Subject prematurely | 14 | (30.4) | 11 | (45.8) |
| terminated the study | | | | |
| Received 40 mg DR-6MP, | 2 | (4.3) | | |
| excluded | | | | |
| Never started treatment, | 4 | (8.7) | | |
| excluded | | | | |

Fourteen subjects (30.4%) from the DR-6MP treatment arm and 11 subjects (45.8%) from the PURINETHOL® treatment arm withdrew early from the study. Of these, 10 subjects (25.0%) and 7 subjects (29.2%) from the DR-6MP and PURINETHOL® treatment arms, respectively, withdrew due to AEs; one subject (4.2%) from the PURINETHOL® treatment arm withdrew due to non-compliance; one subject from each treatment arm was lost to follow-up; 3 subjects (7.5%) and 2 subjects (8.3%) from the DR-6MP and PURINETHOL® treatment arms, respectively, withdrew their consent for participation in the study (Table 6).

TABLE 6
Reasons for early discontinuation

| | DR-6MP 80 mg | | PURINETHOL ® | |
|---|---|---|---|---|
| Reason for | N = 40 | | N = 24 | |
| Discontinuation of Study | N (%) | | N (%) | |
| Adverse Events | 10 | (25.0) | 7 | (29.2) |
| Subject non-compliance | — | — | 1 | (4.2) |
| Lost to Follow Up | 1 | (2.5) | 1 | (4.2) |
| Consent Withdrawal | 3 | (7.5) | 2 | (8.3) |

### Data Sets Analyzed

The following populations were defined for statistical analyses:
Intent to Treat (ITT) population: 64 subjects (40 subjects in the DR-6MP treatment arm and 24 subjects in the PURINETHOL® treatment arm; Table 7).
Safety population was defined as the ITT population.
Per Protocol Population (PP): 37 subjects (25 subjects in the DR-6MP treatment arm and 12 subjects in the PURINETHOL® treatment arm; Table 7).
Modified Intent To Treat Population (mITT) includes all PP subjects as well as those patients who dropped out at/after week 6 (visit 7) and at/after week 8 (visit 8) with Last Observation Carried Forward (LOCF) as their final observation. The populations was named mITT1 (Week 6) and mlTT2 (Week 8).

TABLE 7
Study analyzed populations

| | DR-6MP 80 mg | PURINETHOL ® | All |
|---|---|---|---|
| Study Population | N | N | N |
| ITT | 40 | 24 | 64 |
| PP | 25 | 12 | 37 |

### Demographic and Other Baseline Characteristics

The demographic and baseline characteristics of the study subjects were similar between both treatment arms (Table 8). All study subjects were Caucasian except for one subject from the DR-6MP treatment arm who was black. The average age of the study subjects at screening was 35.5±11.4 years (range: 18.4-54.6) in the DR-6MP treatment arm and 33.7±12.5 years (range: 19-64.1) in the PURINETHOL® treatment arm. Nineteen subjects (47.5%) from the DR-6MP treatment arm and 15 subjects (62.5%) from the PURINETHOL® treatment arm were female. This female predominance in CD was documented in previous incidence studies. Most of the female subjects were pre-menopausal and all pre-menopausal women used contraceptive methods.

TABLE 8
Baseline characteristics of the study population

| | DR-6MP 80 mg | PURINETHOL ® | |
|---|---|---|---|
| Parameter | N = 40 | N = 24 | P value* |
| Mean Age (years) ± SD | 35.5 ± 11.4 | 33.7 ± 12.5 | 0.540 |
| Range (years) | 18.4-54.6 | 19.0-64.1 | |
| Gender: | | | |
| Male, N (%) | 21 (52.5) | 9 (37.5) | 0.244 |
| Female, N (%) | 19 (47.5) | 15 (62.5) | |
| Race: | | | |
| Caucasian, N (%) | 39 (97.5) | 24 (100) | 0.435 |
| Black, N (%) | 1 (2.5) | — | |

SD = standard deviation
*P value by T-test for continuous variables and by Chi-square test for categorical values

### CD History

The mean age at diagnosis of CD was 27.6±11.6 years (range 8.6-53.7) for subjects in the DR-6MP treatment arm and 28.2±13.1 years (range 10.8-61.9) for subjects in the PURINETHOL® treatment arm. A higher percent of patients in the DR-6MP treatment arm compared with the PURINETHOL® treatment arm were newly diagnosed patients (37.5% vs. 25.0%, respectively) as well as patients who have had the disease for more than 10 years (35.0% vs. 20.8%, respectively; Table 9).

TABLE 9
Time from disease diagnosis at baseline

| | DR-6MP 80 mg, N = 40 | PURINETHOL ®, N = 24 | P value* |
|---|---|---|---|
| Mean age at diagnosis of | 27.6 ± 11.6 | 28.2 ± 13.1 | 0.855 |
| CD (years) ± SD | | | |
| Median (Range), years | 24.4 (8.6-53.7) | 26.3 (10.8-61.9) | |
| Disease duration, N (%) | | | |
| ≦1 year | 15 (37.5) | 6 (25.0) | 0.1557 |
| >1 year, ≦5 years | 5 (12.5) | 8 (33.3) | |
| >5 years, ≦10 years | 6 (15.0) | 5 (20.8) | |
| >10 years | 14 (35.0) | 5 (20.8) | |

A similar proportion of patients in the DR-6MP treatment arm and the PURINETHOL® treatment arm were treated concomitantly with 5-aminosalycylic acid (5-ASA) (35.0% and 37.5%, respectively). One patient in the DR-6MP treatment arm was antibiotic-dependent and 5 patients from each treatment arm (12.5% of patients in the DR-6MP treatment arm and 20.8% of patients in the PURINETHOL® treatment arm) were steroid-dependent (Table 10).

TABLE 10
Concomitant CD-related medications at baseline

| | DR-6MP 80 mg, N = 40 | PURINETHOL ®, N = 24 |
|---|---|---|
| | N (%) | N (%) |
| 5-ASA | 14 (35.0) | 9 (37.5) |
| Antibiotics | 1 (2.5) | 0 (0) |
| Steroids | 5 (12.5) | 5 (20.8) |

### Measurements of Treatment Compliance

The overall compliance rate was 97.4%±4.9 (N=35) in the DR-6MP treatment arm and 97.5%±4.2 (N=23) in the PURINETHOL® treatment arm.

### Efficacy Evaluation

### Analysis of Efficacy

### Primary Efficacy Endpoint

### CDAI Score During the Study—ITT Population

The ITT population included all 64 randomized/enrolled patients (40 subjects in the DR-6MP treatment arm and 24 subjects in the PURINETHOL® treatment arm; Table 7) who received a subject study number, signed the ICF and received at least one dose of study medication.

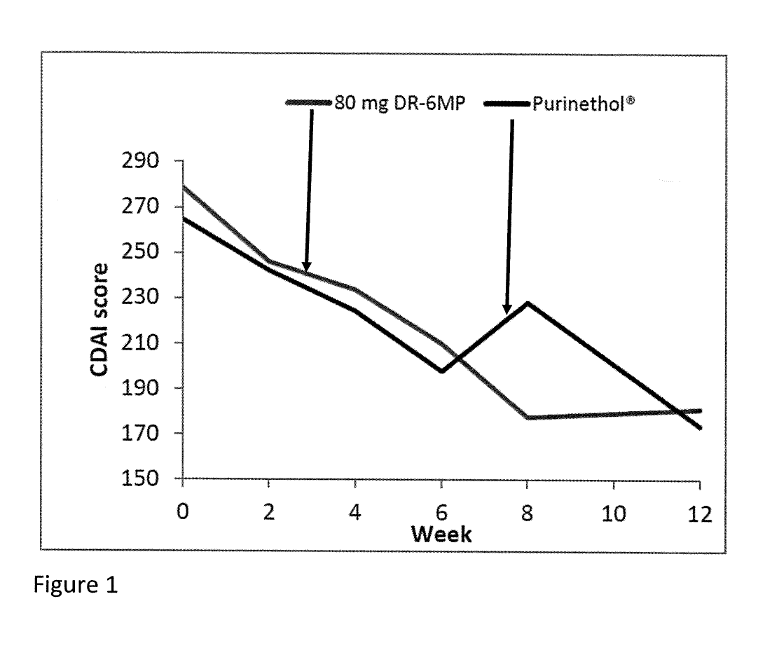

The average CDAI score of the ITT population during study visits is displayed in Table 11 and FIG. 1. A decrease in CDAI score from baseline was observed in both treatment arms. At week 8, a significant difference in CDAI score was observed between the treatment groups (p=0.0178 by ANCOVA adjusted for baseline CDAI).

TABLE 11
CDAI score during the study (ITT population)
CDAI Score

| | DR-6MP 80 mg | | | PURINETHOL ® | | | |
|---|---|---|---|---|---|---|---|
| | N | Mean ± SD | 95% CI | N | Mean ± SD | 95% CI | P value* |
| Randomization | | 286.0 ± 54.4 | 268.6-303.4 | 24 | 274.8 ± 44.1 | 256.2-293.5 | |
| Baseline | 40 | 278.6 ± 61.8 | 258.2-298.9 | 24 | 264.6 ± 48.2 | 244.2-284.9 | |
| Week 2 | 33 | 246.0 ± 94.1 | 212.6-279.4 | 22 | 242.0 ± 70.9 | 210.6-273.5 | |
| Week 4 | 30 | 233.7 ± 81.1 | 203.4-264.0 | 19 | 224.3 ± 74.3 | 188.5-260.1 | |
| Week 6 | 29 | 210.0 ± 89.3 | 176.0-244.0 | 17 | 197.7 ± 76.6 | 158.3-237.1 | |
| Week 8 | 29 | 177.7 ± 88.0 | 144.2-211.1 | 14 | 228.1 ± 43.8 | 202.9-253.4 | 0.0178 |
| Week 12 | 29 | 181.1 ± 109.0 | 138.8-223.4 | 13 | 173.8 ± 92.2 | 118.1-229.5 | 0.8712 |

SD = standard deviation,
CI = confidence interval
*P value for the difference between treatment groups by ANCOVA adjusted for baseline CDAI

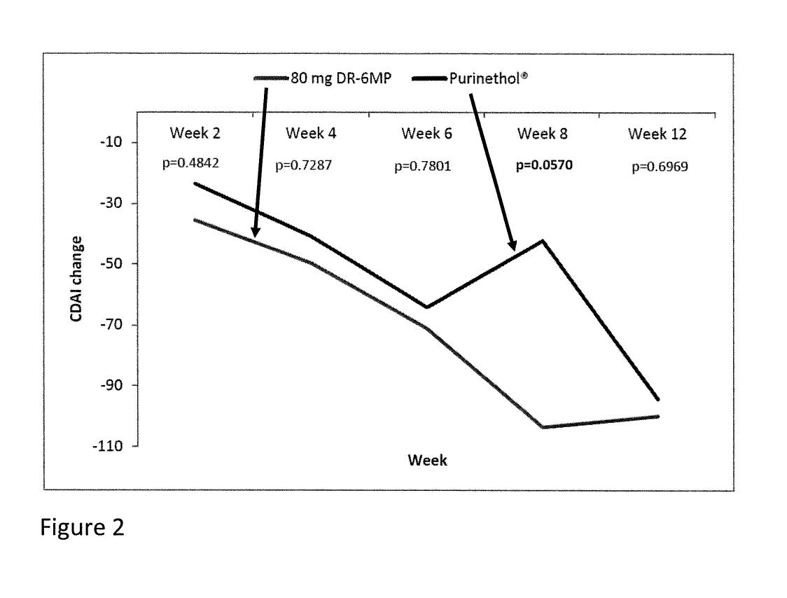

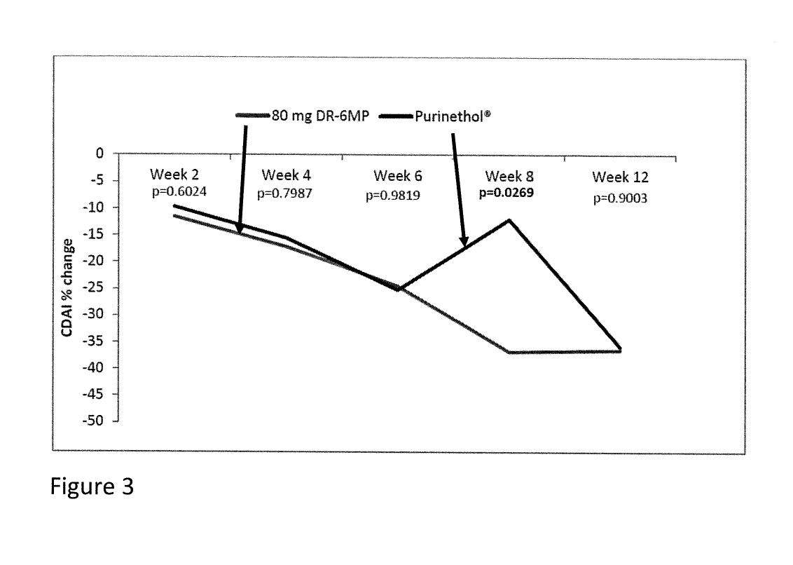

In the DR-6MP treatment arm, a statistically significant relative decrease in CDAI score was observed from baseline to week 8 (−36.7%±24.5, p<0.0001), and from baseline to week 12 (−36.4%±32.6, p<0.0001). In the PURINETHOL® treatment arm, the relative decrease in CDAI score from baseline to week 8 was not statistically significant, but the relative decrease from baseline to week 12 was statistically significant (−35.8%±31.2, p=0.0034). Comparison between the two treatment arms showed that CDAI change and the relative change from baseline were similar during all study visits except for week 8 in which the change and relative change in CDAI score were greater in the DR-6MP treatment arm compared with that of the PURINETHOL® treatment arm (p=0.0424 and p=0.0130 by ANCOVA adjusted for baseline CDAI, age, gender and baseline weight for the differences in absolute changes and relative changes, respectively, in CDAI score from Baseline to week 8; Table 12 and FIGS. 2 and 3).

TABLE 12
CDAI Score change from Baseline (ITT population)

| | CDAI Score | | | | | |
|---|---|---|---|---|---|---|
| | DR-6MP 80 mg | | PURINETHOL ® | | | |
| | N | Mean ± SD | N | Mean ± SD | P value** | P value*** |
| Change at week 8 | 29 | −103.5 ± 74.8 | 14 | −42.2 ± 71.0 | 0.0570 | 0.0424 |
| (P value*) | | (<0.0001) | | (0.0494) | | |
| % Change at week 8 | 29 | −36.7 ± 24.5 | 14 | −12.0 ± 28.8 | 0.0269 | 0.0130 |
| (P value*) | | (<0.0001) | | (0.1040) | | |
| Change at week 12 | 28 | −99.8 ± 89.6 | 13 | −94.2 ± 83.9 | 0.6969 | 0.9789 |
| (P value*) | | (<0.0001) | | (0.0034) | | |
| % Change at week 12 | 28 | −36.4 ± 32.6 | 13 | −35.8 ± 31.2 | 0.9003 | 0.9881 |
| (P value*) | | (<0.0001) | | (0.0034) | | |

SD = standard deviation
*P value by signed rank test for the statistical significance of CDAI change within treatment
**P value by non-parametric Wilcoxon test for the statistical significance of the difference in CDAI change between treatments
***P value by ANCOVA adjusted for baseline CDAI, age, gender and baseline weight for the statistical significance of the difference in CDAI change between treatments

### CDAI Score During the Study—PP Population

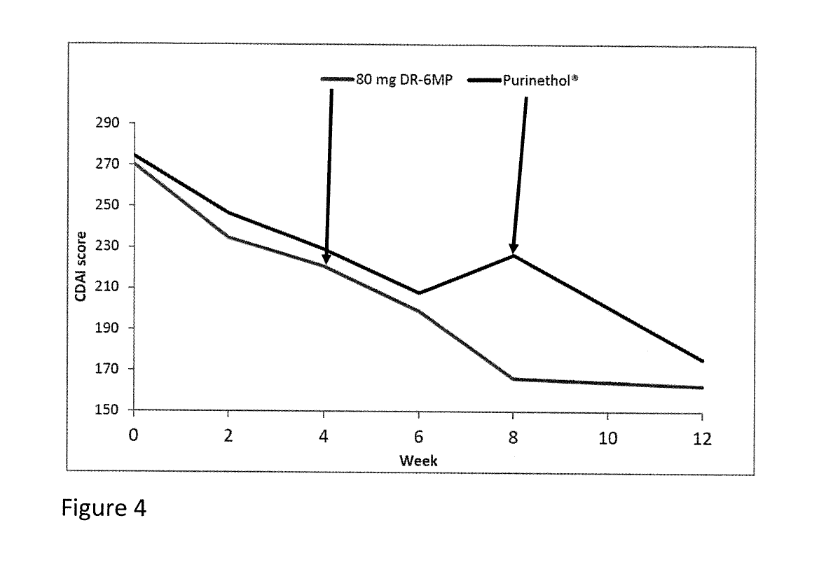

The average CDAI score of the PP population during study visits is displayed in FIG. 4. A decrease in CDAI score from baseline was observed in both treatment arms.

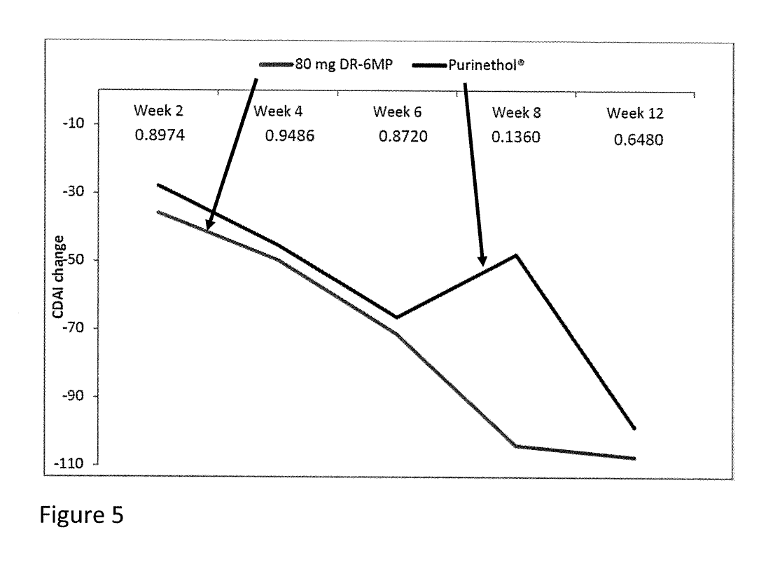

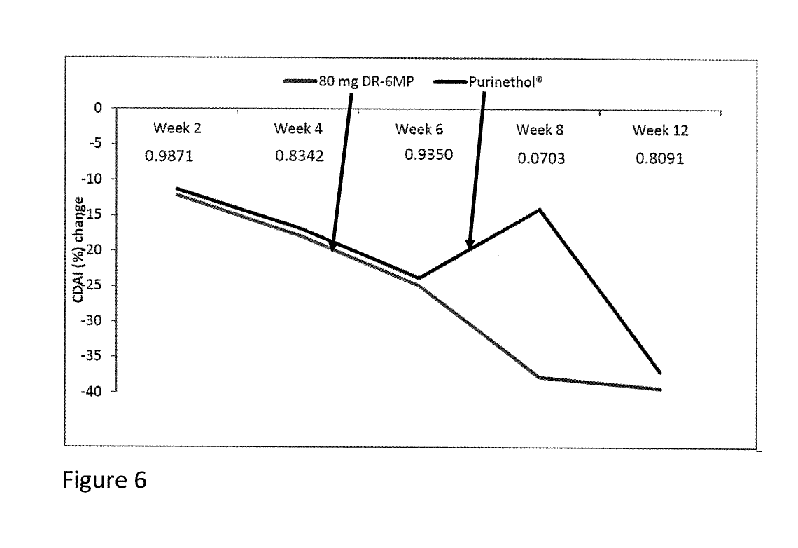

The change in CDAI score from baseline to each study visit of the PP population is presented in FIG. 5, and the relative change in CDAI score from baseline to each study visit of the PP population is presented in FIG. 6. Comparison between the two treatment arms showed that CDAI change and the relative change from baseline of the PP population were similar during all study visits except for week 8 in which the difference in relative change in CDAI score from baseline showed a trend for a greater change in the DR-6MP treatment arm compared with that of the PURINETHOL® treatment arm (p=0.0703; FIG. 6).

### Primary Efficacy Endpoint

The Proportion of Subjects with Clinical Response at Week 12—ITT population

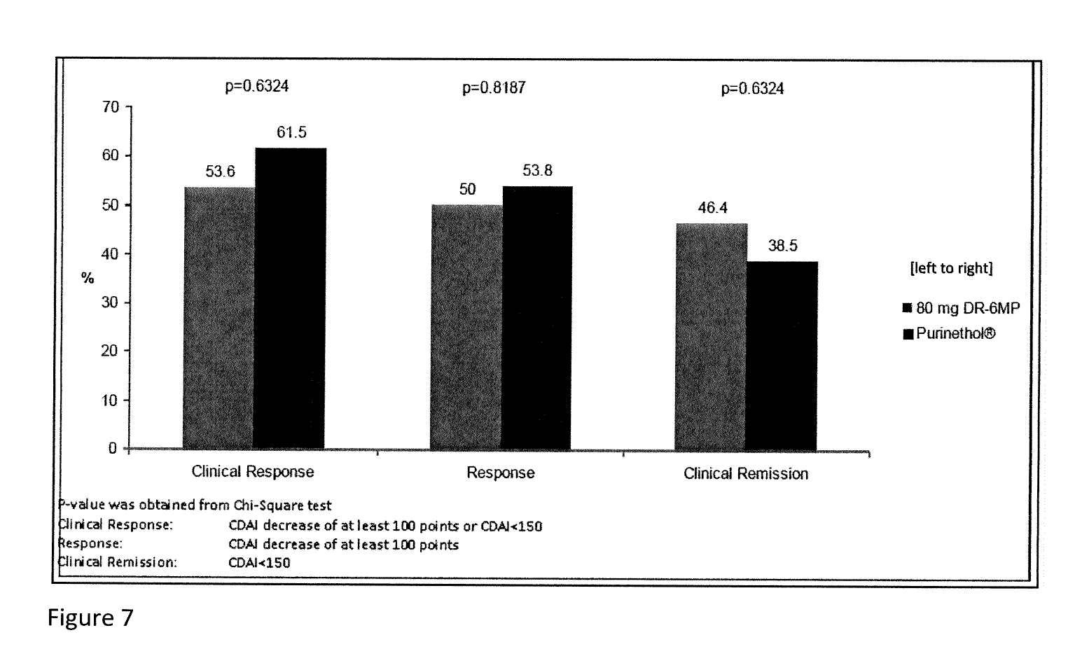

A similar proportion of subjects from the DR-6MP and from the PURINETHOL® treatment arms had clinical response (53.6% and 61.5%, respectively, p=0.6324), response (50.0% and 53.8%, respectively, p=0.187) and remission (46.4% and 38.5%, respectively, p=0.6324) after 12 weeks of treatment (Table 13 and FIG. 7), demonstrating clinical non-inferiority of DR-6MP 80 mg relative to PURINETHOL®.

TABLE 13
The proportion of subjects with
clinical response at week 12 (ITT population)

| | DR-6MP 80 mg | | PURINETHOL ® | | |
|---|---|---|---|---|---|
| | N (%) | 95% CI | N (%) | 95% CI | P value* |
| Clinical response | 15 (53.6) | 0.351- | 8 (61.5) | 0.351- | 0.6324 |
| Response | 14 (50.0) | 0.720 | 7 (53.8) | 0.880 | 0.8187 |
| Remission | 13 (46.4) | | 5 (38.5) | | 0.6324 |

*P value by Chi-square for the difference between treatment groups

The Proportion of Subjects with Clinical Response at Week 12—PP Population

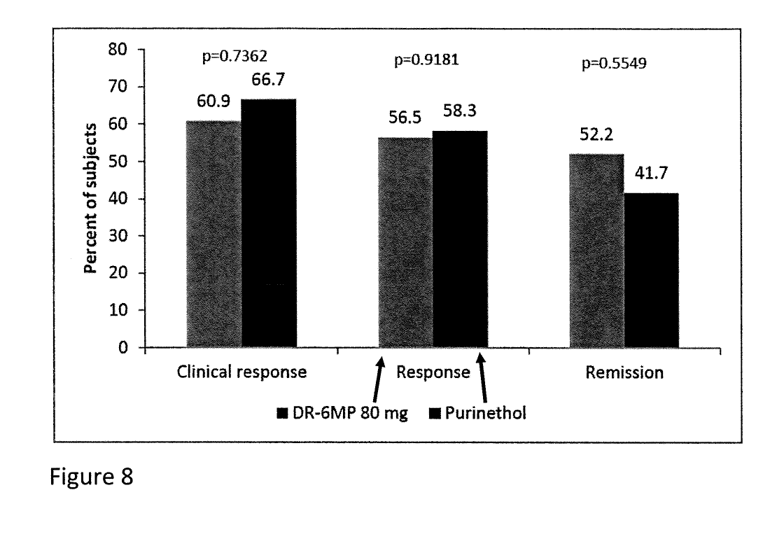

A similar proportion of subjects in the DR-6MP and in the PURINETHOL® treatment arms had clinical response (60.9% and 66.7%, respectively), response (56.5% and 58.3%, respectively) and remission (52.2% and 41.7%, respectively) after 12 weeks of treatment (Table 14 and FIG. 8).

The Proportion of Subjects with Clinical Response at Week 12—mITT1 Population

TABLE 14
The proportion of subjects
with clinical response at week 12 (PP population)

| | DR-6MP 80 mg | PURINETHOL ® | |
|---|---|---|---|
| | N (%) | N (%) | P value* |
| Clinical response | 14 (60.9) | 8 (66.7) | 0.7362 |
| Response | 13 (56.5) | 7 (58.3) | 0.9181 |
| Remission | 12 (52.2) | 5 (41.7) | 0.5549 |

*P value by Chi-square for the difference between treatment groups

The mITT1 population was defined as the PP population and patients who dropped out at/after week 6 with LOCF as their final observation.

The same proportion of subjects from the DR-6MP and from the PURINETHOL® treatment arms had clinical response (62.5%), and a similar proportion had response (58.3% and 56.3%, respectively) and remission (50.0% and 43.8%, respectively) after 12 weeks of treatment (Table 15).

TABLE 15
The proportion of subjects with clinical
response at week 12 (mITT1 population)

| | DR-6MP 80 mg | PURINETHOL ® | |
|---|---|---|---|
| | N (%) | N (%) | P value* |
| Clinical response | 15 (62.5) | 10 (62.5) | 1.00 |
| Response | 14 (58.3) | 9 (56.3) | 0.8961 |
| Remission | 12 (50.0) | 7 (43.8) | 0.6982 |

*P value by Chi-square for he difference between treatment groups

The Proportion of Subjects with Clinical Response at Week 12—mITT2 population

The mITT2 population was defined as the PP population and patients who dropped out at/after week 8 with LOCF as their final observation.

A similar proportion of subjects from the DR-6MP and from the PURINETHOL® treatment arms had clinical response (62.5% and 61.5%, respectively), response (58.3% and 53.8%, respectively) and remission (50.0% and 38.5%, respectively) after 12 weeks of treatment (Table 16).

TABLE 16
The proportion of subjects with
clinical response at week 12 (mITT2 population)

| | DR-6MP 80 mg | PURINETHOL ® | |
|---|---|---|---|
| | N (%) | N (%) | P value* |
| Clinical response | 15 (62.5) | 8 (61.5) | 0.9541 |
| Response | 14 (58.3) | 7 (53.8) | 0.7925 |
| Remission | 12 (50) | 5 (38.5) | 0.5014 |

*P value by Chi-square for he difference between treatment groups

### Secondary Efficacy Endpoints

### Time to Clinical Response—ITT Population

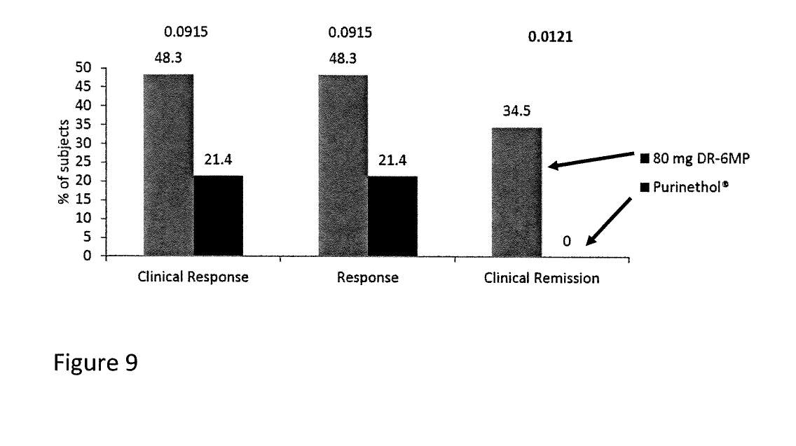

The proportion of subjects who first achieved clinical response (CDAI reduction by 100 points or CDAI score <150) was similar in weeks 2, 4, 6 and 12 in both treatment arms. In week 8, a trend was observed for a larger proportion of subjects with clinical response in the DR-6MP treatment arm compared with the PURINETHOL® treatment arm (48.3% vs. 21.4%, respectively, p=0.0915; Table 17, FIG. 9). Therefore, DR-6MP induced a clinical effect 4 weeks earlier than PURINETHOL®.

TABLE 17
The proportion of subjects with clinical response by week
and treatment (ITT population)

| | DR-6MP 80 mg | PURINETHOL ® | |
|---|---|---|---|
| Week | N (%) | N (%) | P value* |
| 2 | 5 (15.2) | 2 (9.1) | 0.5088 |
| 4 | 8 (26.7) | 5 (26.3) | 0.9332 |
| 6 | 11 (37.9) | 6 (35.3) | 0.8581 |
| 8 | 14 (48.3) | 3 (21.4) | 0.0915 |
| 12 | 15 (53.6) | 8 (61.5) | 0.6324 |

*P value by Chi-square for the difference between treatment groups

### Time to Response—ITT Population

The proportion of subjects who first achieved response (CDAI reduction by 100 points) was similar in weeks 4, 6 and 12 in both treatment arms. In weeks 2 and 8, a trend was observed for a larger proportion of subjects with response in the DR-6MP treatment arm compared with the PURINETHOL® treatment arm (15.2% vs. 0%, respectively at week 2, p=0.0515; 48.3% vs. 21.4%, respectively at week 8, p=0.0915; Table 18, FIG. 9). Therefore, DR-6MP induced an earlier response compared with PURINETHOL®.

TABLE 18
The proportion of subjects with response
by week and treatment (ITT population)

| | DR-6MP 80 mg | PURINETHOL ® | |
|---|---|---|---|
| Week | N (%) | N (%) | P value* |
| 2 | 5 (15.2) | 0 (0) | 0.0515 |
| 4 | 7 (23.3) | 3 (15.8) | 0.5232 |
| 6 | 11 (37.9) | 5 (29.4) | 0.5582 |
| 8 | 14 (48.3) | 3 (21.4) | 0.0915 |
| 12 | 14 (50.0) | 7 (53.8) | 0.8187 |

*P value by Chi-square for the difference between treatment groups
Response: CDAI reduction by 100 points

### Time to Clinical Remission (CDAI Score <150) at Week 2, 4, 6, 8 and 12—ITT Population

The proportion of subjects with clinical remission (CDAI score <150) was similar in weeks 2, 4, 6 and 12 in both treatment arms. At week 8, the proportion of subjects with clinical remission was statistically significantly greater in the DR-6MP treatment arm compared with the PURINETHOL® treatment arm (34.5% vs. none, respectively, p=0.0121; Table 19 and FIG. 9).

TABLE 19
The proportion of subjects with clinical remission (CDAI score <
150) by week and treatment (ITT population)

| | DR-6MP 80 mg | PURINETHOL ® | |
|---|---|---|---|
| Week | N (%) | N (%) | P value* |
| 2 | 4 (12.1) | 2 (9.1) | 0.7240 |
| 4 | 3 (10.0) | 3 (15.8) | 0.5469 |
| 6 | 7 (24.1) | 4 (23.5) | 0.9627 |
| 8 | 10 (34.5) | — | 0.0121 |
| 12 | 13 (46.4) | 5 (38.5) | 0.6324 |

*P value by Chi-square for the difference in proportion of Clinical Remission between treatments
Clinical remission: CDAI score < 150

### The Proportion of Patients Maintaining Remission for Two Consecutive Visits

The proportion of patients maintaining remission (i.e., CDAI<k50 maintained for 2 weeks) within the period of about 4 to 8 weeks, based on the pharmacodynamics properties of the test drug, is an appropriate primary endpoint to justify short-term treatment of active CD.

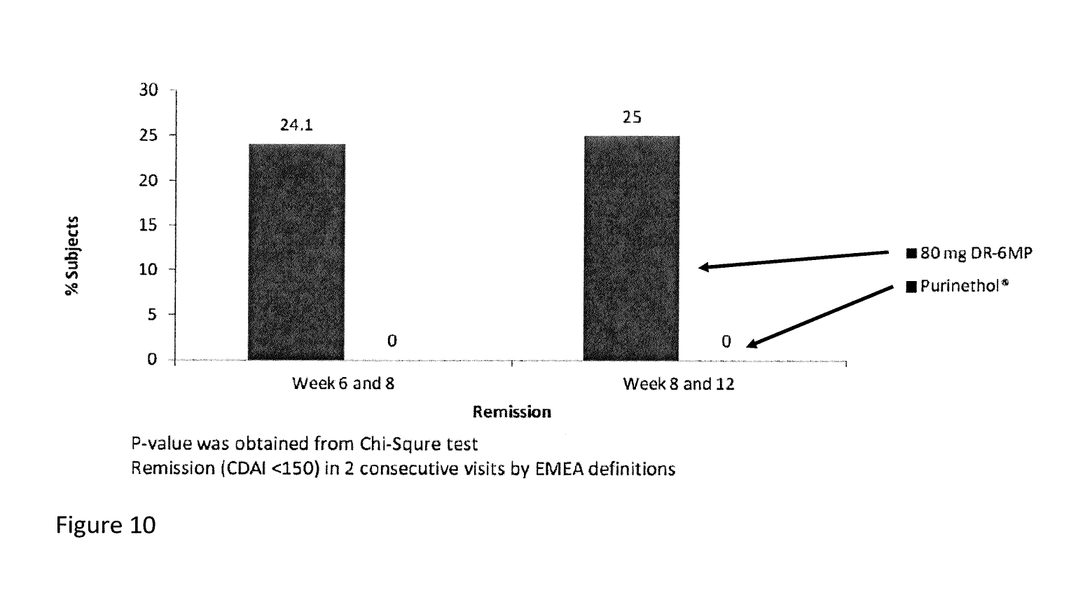

As displayed in the table below, the proportion of patients in the DR-6MP treatment arm who achieved remission for two consecutive visits—on week 6 and 8 as well as on weeks 8 and 12—was statistically significantly higher than the proportion of patients who achieved remission in the PURINETHOL® treatment arm during the same period of time (0.0445 and 0.0477, respectively; Table 20 and FIG. 10).

TABLE 20
Proportion of subjects maintaining remission
(CDAI < 150) for two consecutive visits

| Remission | DR-6MP 80 mg | | PURINETHOL ® | | |
|---|---|---|---|---|---|
| (CDAI < 150) | N | (%) | N | (%) | P value* |
| Weeks 4 & 6 | 1 | (3.4) | 2 | (11.8) | 0.2702 |
| Weeks 6 & 8 | 7 | (24.1) | 0 | (0) | 0.0445 |
| Weeks 8 & 12 | 7 | (25.0) | 0 | (0) | 0.0447 |

*P value by Chi-square test for the statistical significance of the difference between treatments

The Proportion of Subjects Achieving Clinical Remission or Response without Steroid Rescue Therapy at Weeks 4, 6, 8 and 12

Rescue therapy was allowed during the study for those subjects requiring symptomatic relief, as determined by the PI, after at least 2 weeks on the study. Steroids rescue treatment (oral prednisone 40-60 mg/day starting dose) was allowed starting from week 2 and up until week 6, so that following the steroid tapering regimen, all subjects requiring steroids rescue will be steroid-free at the week 12 final visit. Only one subject in the DR-6MP 80 mg treatment arm used the steroid rescue option. An additional subject in the PURINETHOL® treatment arm was prescribed steroid rescue therapy but did not take the steroids. The proportion of subjects achieving clinical remission or response without steroid rescue therapy at weeks 4, 6, 8 and 12, remained the same as for the ITT population: the proportion of subjects who first achieved clinical response was similar in weeks 2, 4, 6 and 12 in both treatment arms. At week 8, a trend was observed for a larger proportion of subjects with clinical response in the DR-6MP treatment arm compared with the PURINETHOL® treatment arm (48.3% vs. 21.4%, respectively, p=0.0915; Table 17, FIG. 9.

The following sections describe analyses of the change in CDAI score from baseline to week 12 in subsets of patients.

Effect of Prior Failure of Treatment with Thiopurines on CDAI Score at Week 12

Patients who experienced serious adverse events on previous thiopurine treatment, (e.g. severe pancreatitis, severe leucopenia, severe hepatotoxicity or bone marrow suppression) were not included in the study to ensure patient safety; as noted in exclusion criteria #17 such patients were to be excluded from any additional 6-mercaptopurine treatment at any dose. However, patients with an experience of previous thiopurine failure, either due to lack of clinical benefit or occurrence of non-serious adverse events, were included in the study. To ensure subject safety, patients with previous thiopurine failure, either due to lack of clinical benefit or occurrence of non-serious adverse events, were assigned only to the DR-6MP treatment arm. This was based on the presumption that DR-6MP is efficacious despite having negligible systemic levels and thus its mechanism of action is different from that of standard 6MP (PURINETHOL®) in which sufficiently high systemic cumulative levels are necessary to obtain efficacy. Therefore, such subjects were only allocated to the DR-6MP treatment group in order to evaluate whether they may benefit from DR-6MP treatment despite previous thiopurine failure.

Patients in the DR-6MP 80 mg treatment arm who had not previously failed thiopurine treatment showed a statistically significant decrease in CDAI score between baseline and week 12 (p=0.0002 for the absolute change and p=0.0001 for the relative change in CDAI score, Table 21). A statistically significant decrease was also observed between baseline and week 12 in the subset of patients who had previously failed thiopurine treatment (p=0.0078, Table 21). Comparison of the change in CDAI score from baseline to week 12 between these two subsets of patients did not show a statistically significant difference between the two groups. The results of this analysis indicate that even patients with previous thiopurine failure could benefit from treatment with DR-6MP.

Of the 11 patients in this subset, 6 had previously been unresponsive to PURINETHOL® in previous attempts at therapy, 1 had been shown to be unresponsive to azathioprine, 1 had stopped prior PURINETHOL® administration due to elevated liver function test results (LFTs), 1 had to lower the dose of their azathioprine therapy due to elevated LFTs, 1 stopped due to an allergic reaction to azathioprine, and 1 had an attack of pancreatitis while on PURINETHOL®.

TABLE 21
Comparison of CDAI Score change from Baseline to week 12 between patients with
previous thiopurine failure and other DR-6MP patients

| | DR-6MP 80 mg | | | | |
|---|---|---|---|---|---|
| | No previous | | | | |
| | thiopurines failure | | Previous thiopurines failure | | |
| CDAI Score | N | Mean ± SD | N | Mean ± SD | P-value** |
| Baseline | 27 | 270.7 ± 48.0 | 11 | 297.7 ± 87.1 | |
| Week 12 | 20 | 155.8 ± 87.8 | 8 | 244.4 ± 135.9 | |
| Change at Week 12 | 20 | −115.4 ± 94.1 | 8 | −60.9 ± 67.5 | 0.0606 |
| (P value*) | | (0.0002) | | (0.0078) | |
| % change at Week 12 | 20 | −41.8 ± 33.8 | 8 | −22.9 ± 26.7 | 0.0907 |
| (P value*) | | (0.0001) | | (0.0078) | |

SD = standard deviation
*P value by signed rank test for the statistical significance of CDAI change within treatment
**P value by non-parametric Wilcoxon test for the statistical significance of the difference in CDAI change between treatments

The group of patients in the thiopurine failure subset who had not experienced a clinical response toP-43 previous thiopurine treatment had their IBDQ scores, relative to baseline 12 weeks after the beginning of administration, rise by an average of 24 (N=3). Among these patients in this group for whom data was available, using the same reference and timeframe, there was a recorded 156% percentage increase in interferon gamma levels (N=1), an 18% increase in CRP levels (N=3), an ESR level decrease of 2% (N=3) and CD62+ levels decreased by 1.8% (N=3). WBC count increased by 1.3% (N=3).

The group of patients in the thiopurine failure subset who experienced an adverse event also saw positive results. One patient with available data had an IBDQ score increase, relative to baseline 12 weeks after the beginning of administration, of 93 points. Avalaible data from patients in this group, using the same reference and timeframe, also showed a decrease of 70% in interferon gamma levels (N=1), a 6% decrease in CRP levels (N=2), a 31% decrease in ESR levels, and a decrease of 1.9% of CD62+ levels (N=2). WBC count increased by 24.8%. These results demonstrate that the benefits of DR-6MP administration are not limited to a single group within the thiopurine failure subset.

CDAI Score Change from Baseline, Patients Treated by any 5-ASA Medications

Patients who were on stable (for at least 2 weeks prior to screening) 5-ASA could remain at that drug dose throughout the study. Although not indicated for CD treatment, 5-ASA compounds are typically used to treat active CD in clinical practice. Due to their limited efficacy, it was unlikely that 5-ASA would interfere with the efficacy assessment in the study.

In this subset of patients, CDAI score decreased (improved) between baseline and 12 weeks in both treatment arms, with a significant decrease in the DR-6MP+5-ASA treatment arm (p=0.0391), and a trend for improvement in the PURINETHOL®+5-ASA treatment arm (p=0.0625). The change in CDAI score between baseline and 12 weeks was not statistically significant in this subset of patients between treatment groups (Table 22).

TABLE 22
Comparison of CDAI Score change from Baseline to week 12 in
patients treated by any 5-ASA medications

| | DR-6MP 80 mg | | PURINETHOL ® | | |
|---|---|---|---|---|---|
| CDAI Score | N | Mean ± SD | N | Mean ± SD | P-value** |
| Baseline | 14 | 300.8 ± 50.9 | 9 | 264.1 ± 47.2 | |
| Week 12 | 9 | 184.9 ± 115.7 | 5 | 135.6 ± 100.4 | |
| Change at Week 12 | 9 | −116.0 ± 109.2 | 5 | −106.5 ± 63.5 | 0.5584 |
| (P value*) | | (0.0391) | | (0.0625) | |
| % change at Week 12 | 9 | −38.5 ± 38.0 | 5 | −47.2 ± 28.5 | 0.7939 |
| (P value*) | | (0.0391) | | (0.0781) | |

SD = standard deviation
*P value by signed rank test for the statistical significance of CDAI change within treatment
**P value by non-parametric Wilcoxon test for the statistical significance of the difference in CDAI change between treatments

For patients in this subgroup, relative to baseline after 12 weeks from the beginning of administration, the available data showed a CDEIS score decrease of 21 (N=20), and an average 33.6 point increase in IBDQ (N=8). These patients also recorded, using the same reference and timeframe, averages of a 15.5% decrease in interferon gamma levels (N=2), a 15.5% decrease in CRP levels (N=2), a 8.9% decrease in ESR levels (N=8), and a 11.2% increase in CD62+ levels (N=8). WBC count decreased by 10.6% (N=10). The data supports the CDAI score results in demonstrating the efficacy of DR-6MP administration for this subgroup.

Comparison of CDAI Score Change from Baseline to Week 12 in Patients Treated by Steroids/Chronic Antibiotic Medications

Subjects considered by the PI to be steroid-dependent or antibiotic-dependent were allowed to enroll in the study on low-dose oral steroids or antibiotics, provided that they were on a stable dose (≧2 weeks prior to screening), and remained on that dose throughout the study. For oral steroids, low-dose is ≦15 mg prednisolone daily or ≦6 mg budesonide daily. The fact that these subjects entered the study with active CD, with CDAI score between 220-450, in spite of constant steroid or antibiotic treatment, indicated that in these subjects, these treatments have not been agents of remission induction. Rather, the clinical efficacy to induce remission or clinical response in these subjects was assessed following the addition of either treatment arm (DR-6MP or PURINETHOL®) as add-on therapy.

A statistically significant decrease in CDAI score between baseline and week 12 was observed in the DR-6MP treatment arm (p=0.0313 for the absolute change and for the relative change), which was not seen in the PURINETHOL® treatment arm. However, the difference between the treatment arms was not statistically significant (Table 23).

TABLE 23
Comparison of CDAI Score change from Baseline to
week 12 in patients treated by steroids/chronic antibiotic medications

| | DR-6MP 80 mg | | PURINETHOL ® | | |
|---|---|---|---|---|---|
| CDAI Score | N | Mean ± SD | N | Mean ± SD | P-value** |
| Baseline | 6 | 319.5 ± 93.2 | 5 | 216.6 ± 31.6 | |
| Week 12 | 6 | 219.8 ± 152.4 | 3 | 181.7 ± 84.3 | |
| Change at Week 12 | 6 | −99.7 ± 84.0 | 3 | −34.7 ± 44.8 | 0.2788 |
| (P value*) | | (0.0313) | | (0.5000) | |
| % change at Week 12 | 6 | −36.1 ± 30.5 | 3 | −18.6 ± 22.3 | 0.3926 |
| (P value*) | | (0.0313) | | (0.5000) | |

SD = standard deviation
*P value by signed rank test for the statistical significance of CDAI change within treatment
**P value by non-parametric Wilcoxon test for the statistical significance of the difference in CDAI change between treatments

For patients in this subset who were adjunctively being administered steroids, relative to baseline after 12 weeks from the beginning of administration, the available data showed a CDEIS score decrease, of 10.8 (N=1), and an average of a 32 point increase in IBDQ (N=3). These patients also recorded, using the same reference and timeframe, a 47% decrease in interferon gamma levels (N=1), and averages of an average of a 49% decrease in CRP levels (N=4), a 36% decrease in ESR levels (N=4), and a 2.3% decrease in CD62+ levels (N=3). WBC count increased by 1.3% (N=4).

For the patient (N=1) in this subset who was adjunctively being administered antibiotics, the data also indicated a benefit. This patient recorded, relative to baseline after 12 weeks from the beginning of administration, a 95 point increase in IBDQ, a 49.7% decrease in interferon gamma levels, a 17.9% decrease in CRP levels, a 25% decease in ESR levels, and an 8% decrease in CD62+ levels. This patient also recorded a 0.67% decrease in WBC, using the same reference and timeframe. The available data demonstrates that adjunctive DR-6MP administration is beneficial for patients who are adjunctively being administered steroids or antibiotics.

### The Change in IBDQ Score Between Baseline and Week 12

The IBDQ evaluates the patient's quality of life using elements of social, systemic and emotional symptoms, as well as bowel related symptoms. A higher score indicates better quality of life.

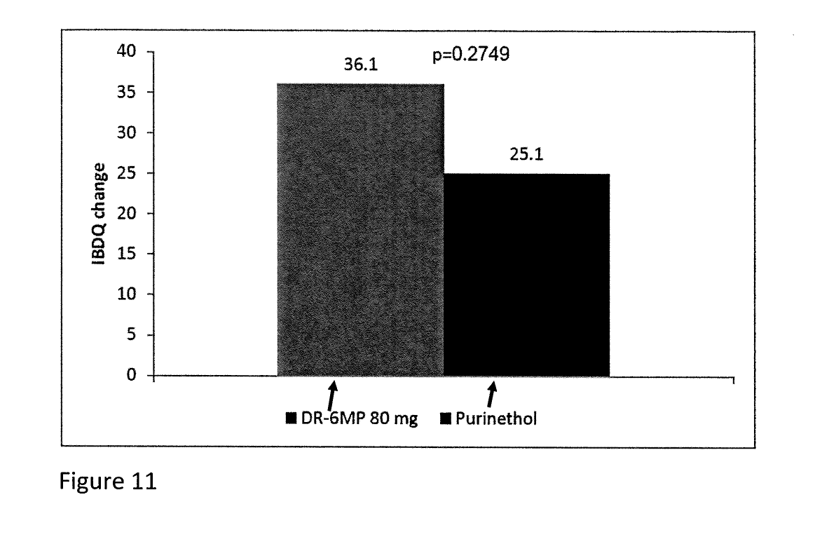

IBDQ score significantly increased between baseline and week 12 in both treatment arms (p<0.0001 and p=0.0134 for DR-6MP and PURINETHOL®, respectively) with a higher increase observed for 6MP, but the extent of increase was similar in both treatment arms (Table 24 and FIG. 11).

TABLE 24
The change in IBDQ score
between baseline and week 12 (ITT population)

| | DR-6MP 80 mg | | PURINETHOL ® | | |
|---|---|---|---|---|---|
| IBDQ Score | N | Mean ± SD | N | Mean ± SD | P-value** |
| Baseline | 40 | 118.4 ± 23.5 | 24 | 129.7 ± 29.0 | 0.1483 |
| Week 12 | 29 | 152.7 ± 38.6 | 15 | 152.9 ± 34.2 | 0.9293 |
| Change at | 29 | 36.1 ± 31.0 | 13 | 25.1 ± 29.0 | 0.2749 |
| Week 12 | | | | | |
| (P-value*) | | (<0.0001) | | (0.0134) | |

SD = standard deviation
*P value by signed rank tes for the statistical significance of IBDQ change within treatment
**P value by non-parametric Wilcoxon test for the statistical significance of the difference in IBDQ change between treatments

Comparison of the Change in IBDQ Score from Baseline to Week 12 by Response to Treatment and Treatment

Comparison of the change in IBDQ score between baseline to week 12 by treatment and response to treatment at week 12 revealed that even non-responders had an improved IBDQ score after 12 weeks of treatment with DR-6MP (p=0.0127) compared with non-responders who were treated with PURINETHOL® (Table 25).

TABLE 25
The change in IBDQ score between baseline and week 12 by
treatment and response to treatment at week 12 (ITT population)

| | IBDQ Score | | | |
|---|---|---|---|---|
| | DR-6MP 80 mg | | PURINETHOL ® | |
| | N | Mean ± SD | N | Mean ± SD |
| Responders at week 12 | | | | |
| Baseline | 15 | 118.9 ± 23.6 | 8 | 129.1 ± 17.8 |
| Week 12 | 15 | 164.8 ± 35.6 | 8 | 169.6 ± 23.6 |
| Change at week 12 | 15 | 45.9 ± 31.5 | 7 | 36.1 ± 31.5 |
| (P value*) | | (0.0003) | | (0.0313) |
| Non-responders at week 12 | | | | |
| Baseline | 13 | 111.8 ± 25.2 | 5 | 117.4 ± 29.7 |
| Week 12 | 12 | 142.2 ± 38.0 | 5 | 127.2 ± 38.2 |
| Change at week 12 | 12 | 26.3 ± 28.0 | 5 | 9.8 ± 23.1 |
| (P value*) | | (0.0127) | | (0.6250) |

SD = standard deviation
*P value by signed rank test for the statistical significance of IBDQ change within treatment

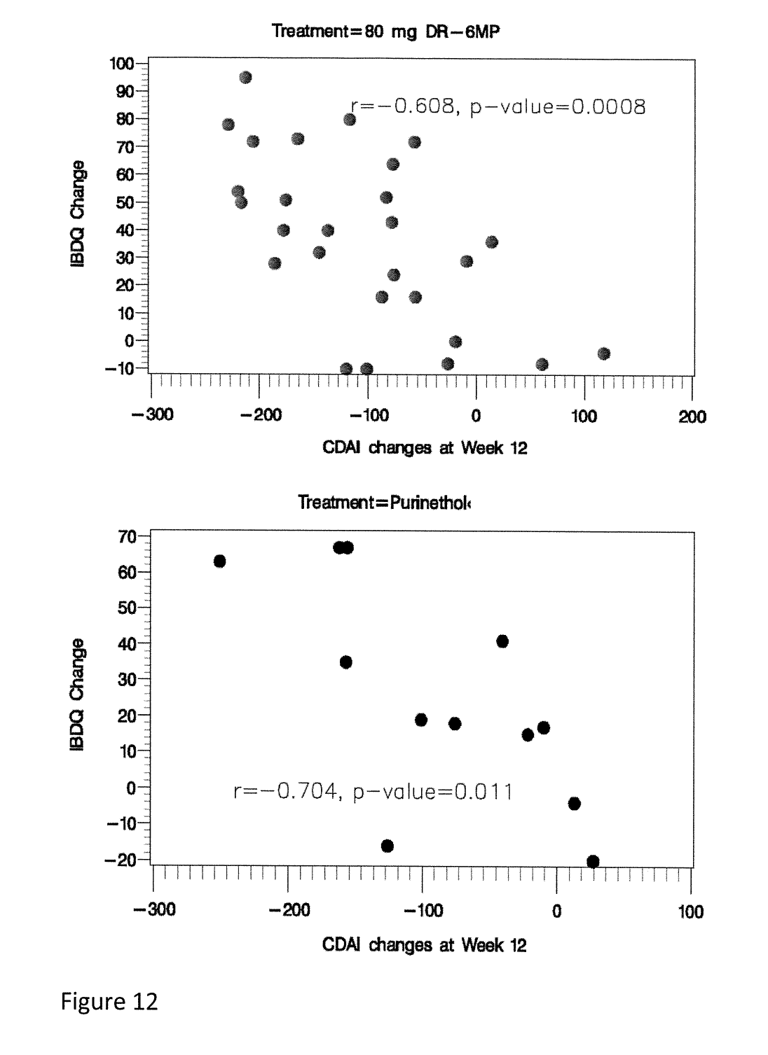

The decrease in CDAI score from baseline to week 12 was associated with an increase in IBDQ score in both treatment arms, i.e., the change in CDAI score between baseline and week 12 was negatively correlated with the change in IBDQ score in both treatment arms (r=−0.6079, p=0.0008 in the DR-6MP treatment arm and r=−0.7036, p=0.0107 in the PURINETHOL® treatment arm; FIG. 12).

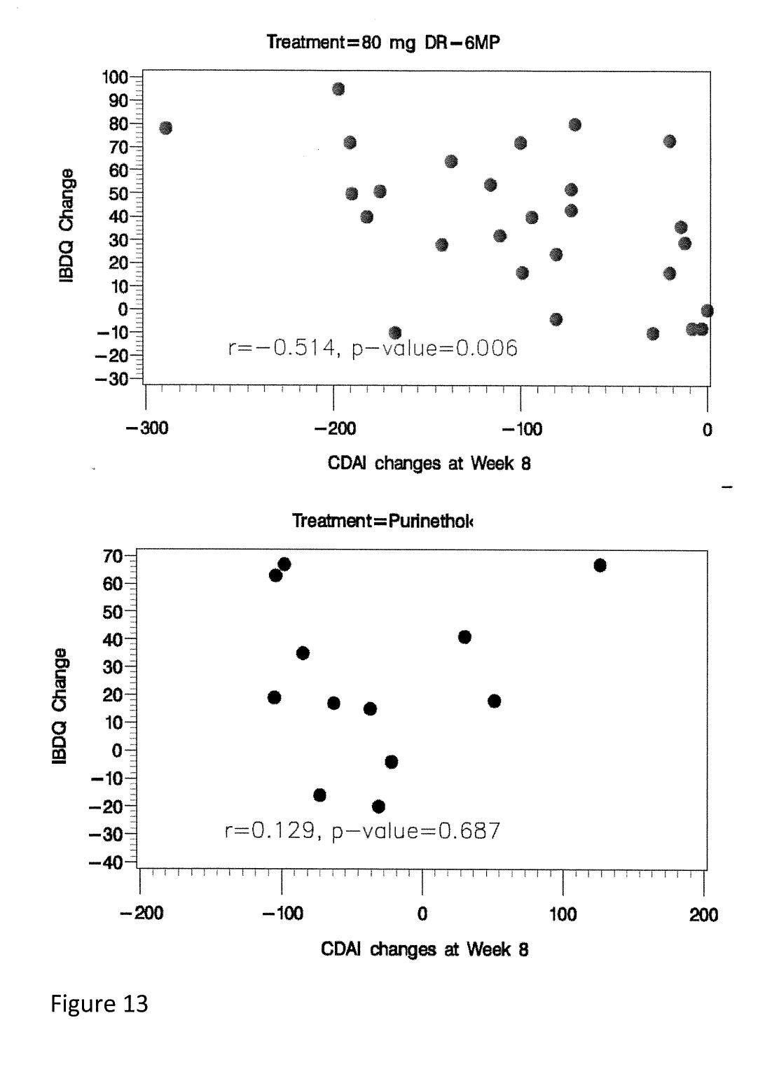

Similarly, the decrease in CDAI score from baseline to week 8 was associated with an increase in IBDQ score in the DR-6MP treatment arm, that is, the change in CDAI score between baseline and week 8 was negatively correlated with the change in IBDQ score (r=−0.5143, p=0.0061). On the other hand, no such correlation was observed in the PURINETHOL® treatment arm (FIG. 13).

### Analysis of the Change in Inflammatory Markers

Changes in immune systemic markers are a measure of efficacy. As PURINETHOL® was expected to have an effect on immune systemic markers, evaluation of the effect of locally-delivered DR-6MP on these markers was performed.

### CRP

CRP is an inflammatory mediator whose blood levels are raised under conditions of acute inflammatory recurrence and rapidly normalize once the inflammation subsides. It may serve as a surrogate marker to monitor inflammatory disease activity and response to treatment.

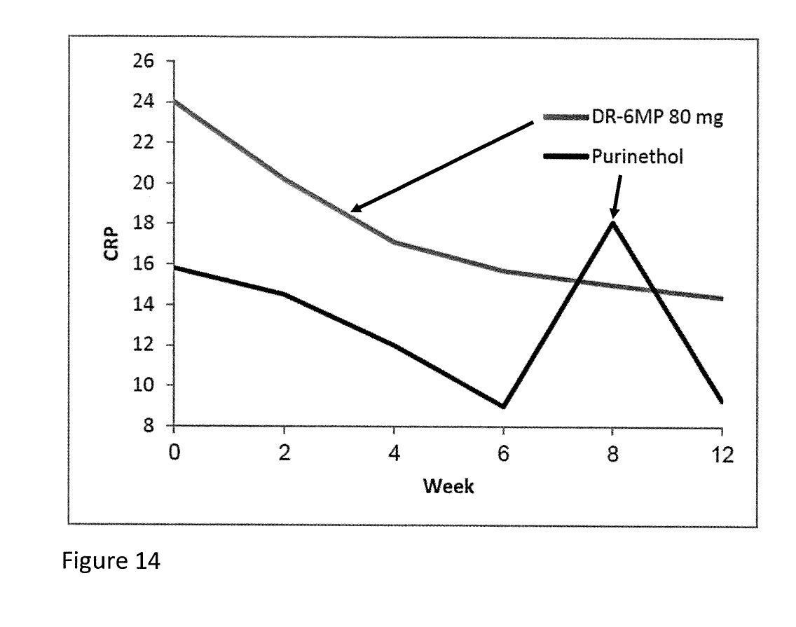

Between Week 6 and Week 12, CRP levels decreased significantly from baseline in the DR-6MP treatment arm. In the PURINETHOL® treatment arm, there was a significant decrease in CRP levels from baseline to each study visit except visit 8. The extent of reduction in CRP levels was similar between the two treatment arms (Table 26 and FIG. 14). These results indicate that although DR-6MP is a locally-delivered drug, it has a similar effect of reducing systemic levels of CRP as the systemically-acting PURINETHOL®.

TABLE 26
Analysis of changes in CRP by treatment and visit (ITT population)

| | CRP | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| Baseline | 40 | 24.0 ± 44.3 | | 24 | 15.8 ± 25.9 | | |
| Change at week 2 | 33 | −3.9 ± 34.9 | 0.3515 | 21 | −2.0 ± 15.4 | 0.0350 | 0.0760 |
| Change at week 4 | 30 | −7.4 ± 33.3 | 0.2351 | 19 | −5.5 ± 10.6 | 0.0095 | 0.1516 |
| Change at week 6 | 28 | −9.8 ± 28.9 | 0.0149 | 17 | −9.4 ± 21.8 | 0.0026 | 0.5231 |
| Change at week 8 | 28 | −10.7 ± 25.7 | 0.0041 | 15 | −2.1 ± 12.1 | 0.3303 | 0.5194 |
| Change at week 12 | 28 | −9.9 ± 31.8 | 0.0572 | 13 | −13.3 ± 31.8 | 0.0398 | 0.1649 |

SD = standard deviation
*P value by signed rank test for the statistical significance of CRP change within treatment
**P value by non-parametric Wilcoxon test for the statistical significance of the difference in CRP change between treatments

### ESR

ESR is a non-specific measure of inflammation used to measure the presence of infection or inflammation and to monitor disease activity.

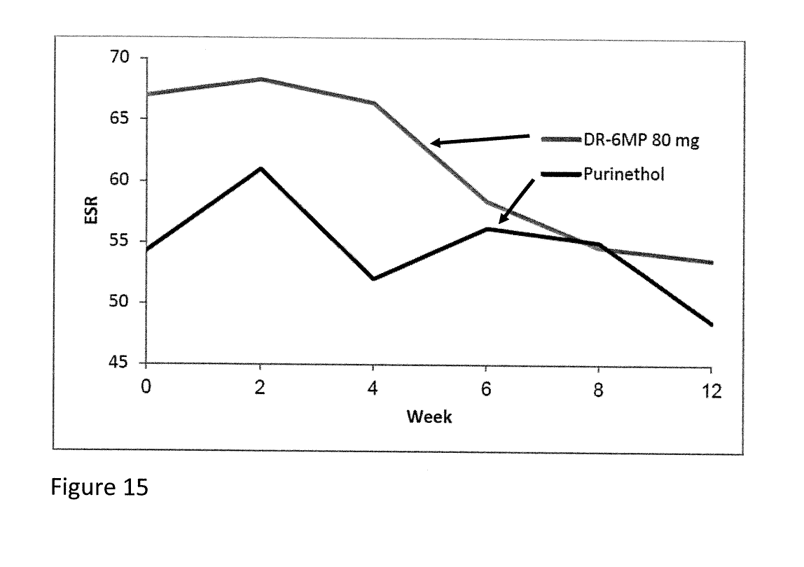

Between Week 6 and Week 12, ESR levels decreased significantly from baseline in the DR-6MP treatment arm, while in the PURINETHOL® treatment arm, there was a significant decrease in ESR levels only from baseline to visit 12. The extent of reduction in ESR levels was similar between the two treatment arms (Table 27 and FIG. 15). Again, this result indicates that the locally-acting DR-6MP has a systemic effect on inflammatory markers.

TABLE 27
Analysis of changes in ESR by treatment and visit (ITT population)

| | ESR | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| Baseline | 39 | 67.0 ± 38.0 | | 23 | 54.3 ± 27.8 | | |
| Change at week 2 | 31 | −0.1 ± 16.2 | 0.9705 | 20 | 2.9 ± 14.8 | 0.3676 | 0.2888 |
| Change at week 4 | 28 | −1.8 ± 18.2 | 0.4962 | 18 | −7.7 ± 18.8 | 0.1419 | 0.4083 |
| Change at week 6 | 27 | −6.7 ± 15.7 | 0.0195 | 16 | −2.8 ± 16.7 | 0.3546 | 0.7172 |
| Change at week 8 | 26 | −12.6 ± 20.9 | 0.0005 | 14 | −6.4 ± 15.8 | 0.1516 | 0.4736 |
| Change at week 12 | 27 | −10.9 ± 20.9 | 0.0170 | 12 | −12.7 ± 15.3 | 0.0225 | 0.6289 |

SD = standard deviation
*P value by signed rank test for the statistical significance of ESR change within treatment
**P value by non-parametric Wilcoxon test for the statistical significance of the difference in ESR change between treatments

### FACS Immunology Parameters and IFN-γ

### IFN-γ Elispot Assay

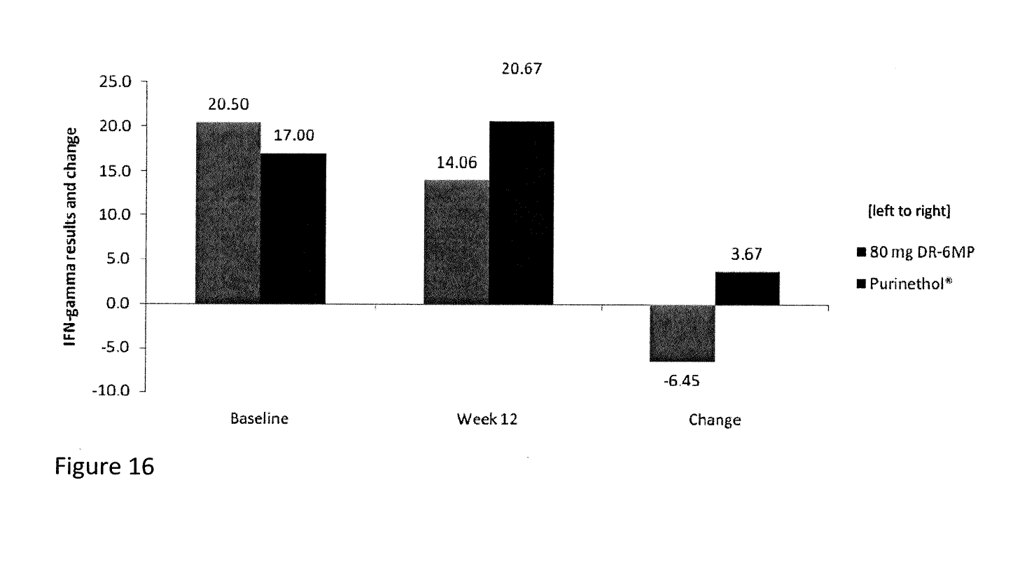

IFN-γ serves as a surrogate marker to monitor immunologic response. As CD patients generally show increased levels of IFN-γ, a reduction indicates improvement in the CD patient's immunological status. The IFN-γ Elispot assay measures the number of T cell clones secreting IFN-γ in response to patient-derived bowel proteins. Therefore, IFN-γ levels could only be evaluated in patients who had undergone a colonoscopy and provided biopsy samples with patient-specific antigens. Due to the small number of such patients in each treatment group, no statistical assessments could be made regarding the change from baseline to visit 12 either within or between treatment arms. It was seen, however, in the small patient sample that IFN-gamma levels decreased in the DR-6MP treatment arm and increased in the PURINETHOL® treatment arm between baseline and week 12 (Table 28 and FIG. 16).

TABLE 28
Change in IFN-gamma-secreting T cell clones from
baseline to week 12 by treatment (ITT population)

| | IFN-gamma | | | |
|---|---|---|---|---|
| | 80 mg DR-6MP | | PURINETHOL ® | |
| | N = 6 | | N = 1 | |
| | Mean ± SD | P-value* | Mean | P-value* |
| Baseline | 20.5 ± 25.8 | | 17.00 | |
| Week 12 | 14.0 ± 7.7 | | 20.67 | |
| Change | −6.5 ± 22.7 | 1.000 | 3.67 | NA |

IFN = interferon,
SD = standard deviation
*P-value from signed rank test indicates the statistical significance of the change within treatment

### FACS Immunology Parameters

Tregs play an important role in the pathogenesis of CD. Tregs actively suppress enteroantigen-reactive cells and contribute to the maintenance of intestinal immune homeostasis. Distinct Treg subsets coexist in the blood and in the intestinal mucosa and have been shown to be important to prevent and/or cure colitis. Failure to control the immune responses disrupts tolerance, and this is proposed to be one of the mechanisms involved in the development of inflammatory bowel disease [Fuss et al. 1996; Foncarolo et al. 2007; Shevach et al. 2006; Ochi et al. 2006]. There is recent evidence from both animal and human studies to indicate that oral administration of low dosage immunomodulatory agents is an effective means for activation of these regulatory T cells and/or alteration of subsets of T cells that are relevant for the pathogenesis of immune-mediated disorders [Schurmann et al. 1995; Ilan et al. PNAS 2010; Ilan et al. JCI 2010; da Cunha et al. 2012; Wu et al. 2009]. In order to determine the immunological effect of the DR-6MP, as compared to PURINETHOL®, FACS analysis was performed on peripheral blood lymphocytes collected at baseline and week 12 for all subjects.

DR-6MP 80 mg resulted in a decrease of CD62+ expression on peripheral T cells as measured by FACS analysis, implying a reduction of lymphocyte adhesion to the site of inflammation. In contrast, PURINETHOL® led to an increase in CD62+ expression.

Treatment with DR-6MP 80 mg also led to decreased expression of CD4+CD25+Foxp3+, and CD3+CD56+, while these parameters increased in the PURINETHOL® treatment arm.

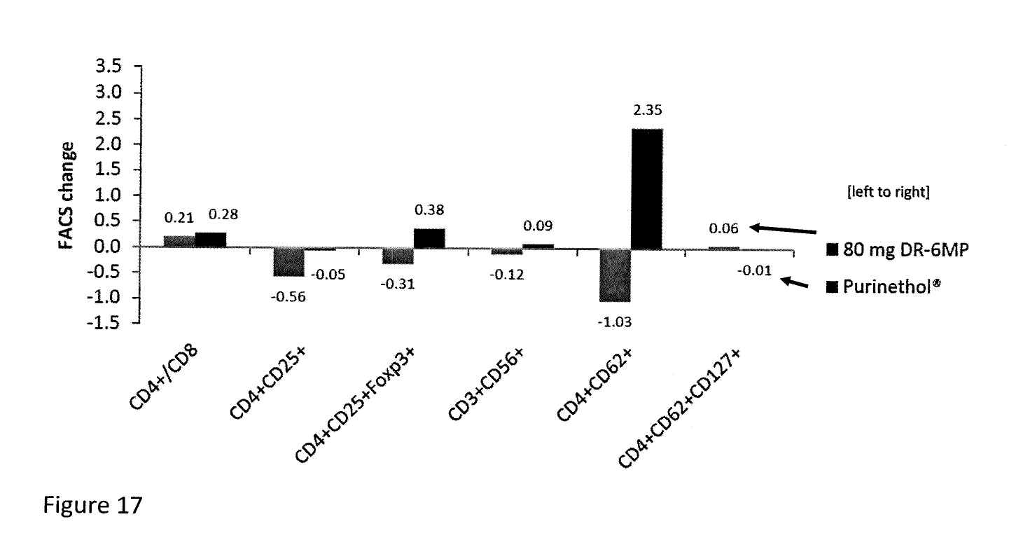

CD4+CD62+CD127+ increased in the DR-6MP treatment arm and decreased in the PURINETHOL® treatment arm. Both treatments led to increased CD4+/CD8+ ratio, with PURINETHOL® resulting in a slightly greater increase compared to DR-6MP. Both treatments resulted in decreased CD4+CD25+ levels with DR-6MP resulting in a greater decrease compared with PURINETHOL® (Table 29 and FIG. 17).

These results indicate that the systemic immunological profile of DR-6MP is different from that of PURINETHOL®, suggesting that its mechanism of action is probably different as well.

TABLE 29
Analysis of the change in FACs immunology parameters between baseline
and week 12 by treatment (ITT population)

| | Change in FACS immunology parameters | | | | |
|---|---|---|---|---|---|
| | 80 mg DR-6MP | | PURINETHOL ® | | |
| | N = 21 | | N = 10 | | |
| | Mean ± SD | P-value* | Mean ± SD | P-value* | P-value** |
| CD4+/CD8+ | 0.21 ± 1.15 | 0.5560 | 0.28 ± 0.62 | 0.2754 | 0.8418 |
| CD4 + CD25+ | −0.56 ± 1.64 | 0.2256 | −0.05 ± 1.94 | 0.6377 | 0.4401 |
| CD4 + CD25 + Foxp3 | −0.31 ± 0.89 | 0.1074 | 0.38 ± 1.17 | 0.2061 | 0.0732 |
| CD3 + CD56+ | −0.12 ± 1.27 | 0.9273 | 0.09 ± 1.06 | 0.8311 | 0.6464 |
| CD4 + CD62+ | −1.03 ± 8.59 | 0.5560 | 2.35 ± 6.12 | 0.3652 | 0.2573 |
| CD4 + CD62 + CD127+ | 0.06 ± 0.28 | 0.8774 | −0.01 ± 0.01 | 0.1250 | 0.3039 |

*P-value by signed rank test indicates the statistical significance of the change within treatment
**P-value by t-test indicates the statistical significance of the difference in the change between the treatments

### Mucosal Healing

The severity of mucosal inflammation, as assessed by colonoscopy/ileoscopy, has been touted as an additional mainstay parameter efficacy assessment in clinical trials. The combination of clinical remission and mucosal healing represents a major goal of CD treatment. Additionally, the necessity for treatment to induce mucosal tissue healing in inflammatory bowel disease in general, has become clinically relevant in light of recent reports correlating disease activity with a patient's overall risk of developing colorectal cancer. Therefore, the assessment of mucosal healing was included as one of the secondary efficacy parameters in the study. Only the subset of subjects who agreed to undergo the procedures at baseline and week 12, under PI consent, were included in the analysis of this parameter.

As a result, only 4 subjects, 3 in the DR-6MP treatment arm and 1 in the PURINETHOL® treatment arm, provided both pre- and post-colonoscopy data for comparison. Another group of subjects, 11 from the DR-6MP treatment arm, and 2 from the PURINETHOL® treatment arm, provided either pre- or post-colonoscopy data. Therefore, because there were so few subjects who were their own controls, and subjects who could only provide pooled data pre- or post-, but not in comparison to their own control, no statistical analysis could be done on the mucosal data, and the results can only be described as detailed below. Table 30 describes the 4 subjects with both pre- and post-colonoscopy data:

TABLE 30
CDEIS of patients who had pre-treatment
and post-treatment colonoscopy

| | CDEIS pre- | CDEIS post- | CDEIS change |
|---|---|---|---|
| Treatment | treatment | treatment | at week 12 |
| DR-6MP 80 mg | 69.5 | 38.0 | −31.5 |
| DR-6MP 80 mg | 37.0 | 40.0 | 3.0 |
| DR-6MP 80 mg | 23.7 | 12.9 | −10.8 |
| PURINETHOL ® | 24.6 | 0 | −24.6 |

For 2 of the 3 subjects treated with DR-6MP, there was evidence of mucosal healing as noted by a reduction in the CDEIS score. In the 1 PURINETHOL® subject, there was also a reduction in CDEIS score following 12 weeks of treatment.

For both treatments, comparison of the subjects' CDEIS scores pre- and post-treatment and of the CDAI scores, shows a correlation, with those subjects showing mucosal healing at week 12, also showing clinical response or remission at week 12, whereas the one subject who did not show evidence of mucosal healing, also did not demonstrate clinical efficacy.

TABLE 31
CDAI scores and clinical status at week 12 of patients
who had pre-treatment and post-treatment colonoscopy

| | CDAI | CDAI | CDAI Change | Clinical Status |
|---|---|---|---|---|
| Treatment | Baseline | Week 12 | at Week 12 | Week 12 |
| DR-6MP 80 mg | 296 | 118 | −178 | CR, Remission |
| DR-6MP 80 mg | 315 | 259 | −56 | Non-responder |
| DR-6MP 80 mg | 353 | 167 | −186 | CR |
| PURINETHOL ® | 193* | 117 | −76 | Remission |

The last subject enrolled with CDAI score at entry, in violation of inclusion criteria of CDAI > 220-450. However, this was not known at the time of randomization as the site erred in the calculation of the randomization CDAI, and only subsequently was this noted during a monitoring session, following completion of the study. Since the subject entered and completed the study in good faith, the data was included for analysis in the ITT population.

It is interesting also to note that when looking at the CDAI data for these subjects at Week 8, the non-responder at week 12 in the DR-6MP group, showed no response at the earlier time-point as well, while those subjects who showed a clinical response (response or remission) at week 12 in the DR-6MP cohort, also showed a clinical response (response or remission) at Week 8. In contrast, although the PURINETHOL® subject showed remission at week 12, there was no clinical response or remission at week 8 for the same subjects (Table 32).

TABLE 32
CDAI scores and clinical status at week 8 of patients who had pre-treatment
and post-treatment colonoscopy

| | CDAI | CDAI | | |
|---|---|---|---|---|
| Treatment | Baseline | Week 8 | CDAI Change at week 8 | Clinical Status Week 8 |
| DR-6MP 80 mg | 296 | 114 | −182 | CR, Remission |
| DR-6MP 80 mg | 315 | 216 | −99 | Non-responder |
| DR-6MP 80 mg | 353 | 211 | −142 | CR |
| PURINETHOL ® | 193* | 244 | 51 | Non-responder |

### Pooled Colonoscopy Data

DR-6MP treatment arm—7 subjects contributed pre-treatment CDEIS score and 4 subjects contributed post-treatment, Week 12 CDEIS score.

PURINETHOL® treatment arm—1 subject contributed pre-treatment CDEIS score and 1 subject contributed post-treatment, Week 12 CDEIS score, as tabulated below in Table 33 and Table 34.

TABLE 33
Pre-treatment CDAI and
CDEIS of patients who had pre-treatment colonoscopy

| Treatment | CDEIS pre-treatment | CDAI baseline |
|---|---|---|
| DR-6MP 80 mg | 65.0 | 253 |
| DR-6MP 80 mg | 40.0 | 258 |
| DR-6MP 80 mg | 48.0 | 288 |
| DR-6MP 80 mg | 0.0 | 304 |
| DR-6MP 80 mg | 24.0 | 282 |
| DR-6MP 80 mg | 188.0 | 399 |
| DR-6MP 80 mg | 44.0 | 247 |
| Mean ± SD | 58.4 ± 60.7 | 290.0 ± 52.3 |
| PURINETHOL ® | 7.0 | 204* |

Last subject entered in compliance with protocol, with randomization CDAI meeting study criterion of >220-450. As per protocol, baseline CDAI was used for the analysis.

TABLE 34
CDAI and CDEIS at week 12
of patients who had post-treatment colonoscopy

| Treatment | CDEIS post-treatment, week 12 | CDAI week 12 |
|---|---|---|
| DR-6MP 80 mg | 29.0 | 213 |
| DR-6MP 80 mg | 14.0 | 183 |
| DR-6MP 80 mg | 0.0 | 49 |
| DR-6MP 80 mg | 17.0 | 116 |
| Mean ± SD | 15.0 ± 11.9 | 140.3 ± 73.1 |
| PURINETHOL ® | 111.0 | 111 |

Looking at the mean pooled data for CDEIS for the 7 DR-6MP subjects pre-treatment (58.4) vs. the mean pooled data for the 4 DR-6MP subjects post-treatment (15.0), there is an intimation of CDEIS improvement. Similarly, looking at the mean pooled CDAI data for these same 7 DR-6MP subjects' pre-treatment (290) vs. the mean pooled data for these same 4 DR-6MP subjects post-treatment (140), there is a comparable intimation of clinical response/remission.

### Changes in Weight and BMI

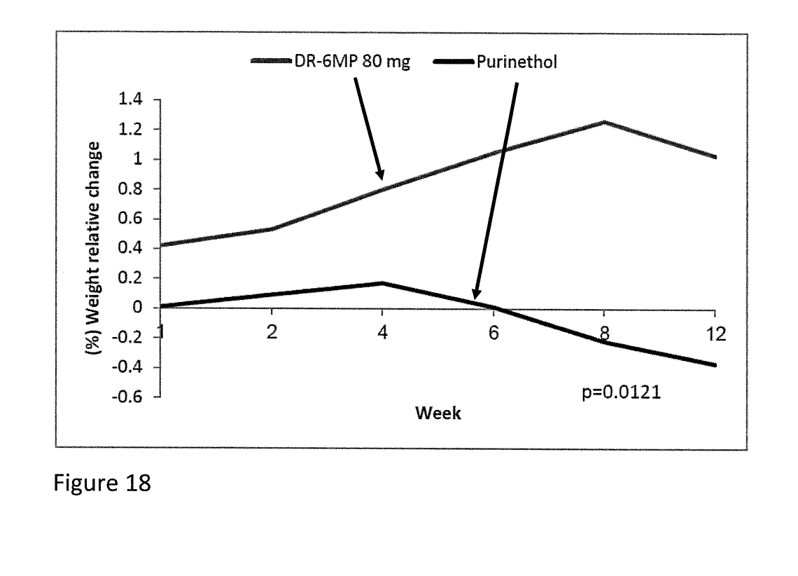

Although weight is measured as part of the vital signs, and is typically included as a safety parameter, in the case of Crohn's disease patients, where weight loss is one of the characteristic features of the disease, a change in the expected weight loss is to be considered a parameter of clinical efficacy. Therefore, the section on changes in weight and BMI have been included in the analyses of clinical efficacy. Weight (Table 35 and FIG. 17) and BMI (Table 36 and FIG. 18) increased in the DR-6MP treatment arm during the 12 weeks of treatment while both parameters decreased in the PURINETHOL® treatment arm. A comparison between the treatment arms of the change in weight and BMI between baseline and each study visit showed a statistically significant difference between the two treatment arms at visit 8 (p=0.0121 by Median test).

TABLE 35
Analysis of changes in weight by treatment and visit (ITT population)

| | Weight | | | | | | |
|---|---|---|---|---|---|---|---|
| | DR-6MP 80 mg | | | PURINETHOL ® | | | |
| | N | Mean ± SD | P value | N | Mean ± SD | P value* | P value** |
| Baseline | 40 | 63.34 ± 11.61 | | 22 | 67.95 ± 16.76 | | |
| Change at week 2 | 34 | 0.37 ± 1.58 | 0.185 | 19 | 0.09 ± 1.33 | 0.752 | 0.6414 |
| Change at week 4 | 30 | 0.56 ± 2.19 | 0.172 | 17 | 0.11 ± 1.57 | 0.774 | 0.9452 |
| Change at week 6 | 29 | 0.70 ± 2.42 | 0.132 | 15 | −0.09 ± 1.71 | 0.834 | 0.1450 |
| Change at week 8 | 28 | 0.85 ± 3.01 | 0.145 | 15 | −0.24 ± 2.96 | 0.758 | 0.0121 |
| Change at week 12 | 29 | 0.65 ± 3.70 | 0.354 | 22 | −0.31 ± 3.95 | 0.763 | 0.7532 |

SD = standard deviation
*P value by paired T-test for the statistical significance of weight change within treatment
**P value by median test for the statistical significance of the difference in weight change between treatments

TABLE 36
Analysis of changes in BMI by treatment and visit (ITT population)

| | BMI | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| Baseline | 40 | 22.65 ± 4.29 | | 22 | 23.94 ± 5.13 | | |
| Change at week 2 | 34 | 0.12 ± 0.56 | 0.206 | 19 | 0.03 ± 0.50 | 0.768 | 0.6414 |
| Change at week 4 | 30 | 0.17 ± 0.72 | 0.196 | 17 | 0.04 ± 0.59 | 0.792 | 0.9452 |
| Change at week 6 | 29 | 0.23 ± 0.80 | 0.136 | 15 | −0.02 ± 0.63 | 0.878 | 0.1450 |
| Change at week 8 | 28 | 0.28 ± 1.00 | 0.151 | 15 | −0.06 ± 1.13 | 0.847 | 0.0121 |
| Change at week 12 | 29 | 0.19 ± 1.22 | 0.399 | 22 | −0.07 ± 1.49 | 0.860 | 0.7532 |

SD = standard deviation,
BMI = body mass index
*P value by paired T-test for the statistical significance of BMI change within treatment
**P value by median test for the statistical significance of the difference in BMI change between treatments

### Efficacy Conclusions

After 12 weeks of treatment, DR-6MP 80 mg was non-inferior to PURINETHOL®. CDAI score decreased to a similar extent in both treatment arms and a similar proportion of subjects achieved clinical response, response and remission. However, a higher proportion of patients in the DR-6MP treatment arm achieved clinical response and clinical remission at week 8, and the change from baseline to week 8 in CDAI score was higher for DR-6MP than for PURINETHOL®. These results imply that patients treated with DR-6MP 80 mg reached a clinical effect 4 weeks earlier than patients treated with PURINETHOL®. Furthermore, a significantly higher proportion of patients in the DR-6MP treatment arm achieved remission for two consecutive visits from week 6 onwards compared with those treated with PURINETHOL®.

As a corollary to the clinical efficacy finding of CDAI, “quality of life” was evaluated using the IBDQ. IBDQ score improved significantly between baseline and week 12 in both treatment arms indicating an improvement in the patients' quality of life with a greater change noted in the DR-6MP group compared with PURINETHOL®. Furthermore, even non-responders to the treatment had an improved IBDQ score after 12 weeks of treatment with DR-6MP (p=0.0127) compared with non-responders treated with PURINETHOL®, whose IBDQ scores did not change significantly from baseline to week 12.

Reviewing the correlation between the CDAI scores at week 8 and the IBDQ score at treatment end revealed that the improvement in IBDQ that was noted in the DR-6MP treatment arm at week 12 was “predicted” by the CDAI improvement observed at Week 8. No such correlation was found for the PURINETHOL® group.

Several of the subjects were treated by adjunctive treatment such as 5-ASA or steroids/chronic antibiotics for the duration of the study concomitantly with the test or reference drug. In both subsets, a significant CDAI decrease from baseline to week 12 was seen in the DR-6MP treatment arm. This indicates that DR-6MP shows efficacy whether given as combination therapy or as monotherapy.

A statistically significant decrease in CDAI score was observed between baseline and week 12 in the subset of patients who had previously failed thiopurine treatment. The results of this analysis indicate that even patients with previous thiopurine failure could benefit from treatment with DR-6MP.

Steroid rescue was needed by one patient from each treatment arm and did not affect the clinical outcome in either treatment arm.

As CD is an immune-related disorder, changes in immunology profile can be correlated with clinical efficacy. A similar decrease in general systemic immune markers, namely, CRP and ESR, was observed between treatment arms, although a greater decrease was noted in the PURINETHOL® arm. This result was expected in the PURINETHOL® treatment arm, but seeing systemic expression by a locally-delivered drug is an important finding.

IFN-γ serves as a surrogate marker to monitor immunologic response. IFN-γ levels, however, could only be evaluated in patients who had done colonoscopy. Due to the small number of such patients in each treatment group, no statistical assessments could be made regarding the change from baseline to visit 12 either within or between treatment arms. However it was demonstrated that the number of T cells secreting IFN-γ in response to the patient-derived bowel proteins decreased in the DR-6MP treatment arm indicating improvement in the CD patients' immunological status, while it increased in the PURINETHOL® treatment arm.

DR-6MP induced a different immunological profile in CD-specific immune markers as measured by FACS analysis than that of PURINETHOL®. For example, DR-6MP led to a decrease of CD62+ expression on peripheral T cells implying a reduction of lymphocyte adhesion to the site of inflammation. In contrast, PURINETHOL® led to an increase in CD62+ expression. Similarly, DR-6MP led to a decrease in CD4+CD25+Foxp3+ and CD3+CD56+ expression, while PURINETHOL® led to an increase in expression of these parameters. The other systemic measures also changed differently between the two groups. The difference in immunological profiles exhibited by the DR-6MP vs. PURINETHOL® underscores that the treatments most likely operate via different mechanisms of action.

Very few patients agreed to undergo colonoscopy and ileoscopy at both time points in the study; therefore statistical analysis of evaluation of mucosal healing at week 12 relative to baseline could not be performed. Of the 4 patients who underwent this evaluation at both time points, improvement in the total CDEIS score was observed in ⅔ subjects in the DR-6MP treatment arm and in one subject in the PURINETHOL® treatment arm. The reduction in CDEIS was correlated with the reduction in CDAI.

Although generally regarded as a safety measure, changes in weight and BMI were included in the efficacy analysis since weight loss is a hallmark of CD. Weight and BMI increased in the DR-6MP treatment arm from baseline to each study visit and decreased in the PURINETHOL® treatment arm.

Moreover, a statistically significant difference between treatment arms was observed at week 8. This data indicates the clinical efficacy of DR-6MP, demonstrating that it has a profound effect on the disease activity by preventing the weight loss that is seen in CD patients.

### Safety Evaluations

### Extent of Exposure

The extent of exposure to the test and reference drugs is displayed in Table 37. Subjects in the DR-6MP received a daily dose of 80 mg while subjects in the PURINETHOL® dose received a median daily dose of 75 mg. Over the course of the 12 week study, the mean exposure per subject during the study to DR-6MP 80 mg was 5.69±2.10 gr and the mean exposure per subject during the study to PURINETHOL® was 4.91±2.90 gr.

TABLE 37
Extent of exposure to the test and reference drugs during the study

| | DR-6MP 80 mg | PURINETHOL ® |
|---|---|---|
| | (N = 35) | (N = 23) |
| Mean ± SD, gr | 5.69 ± 2.10 | 4.91 ± 2.90 |
| Median (range) | 6.72 (0.64-7.28) | 5.53 (0.38-1.12) |

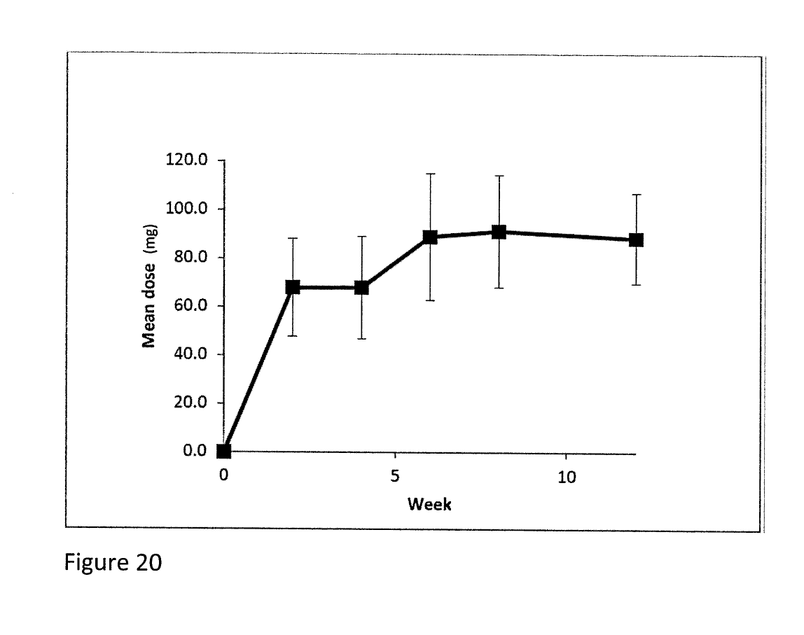

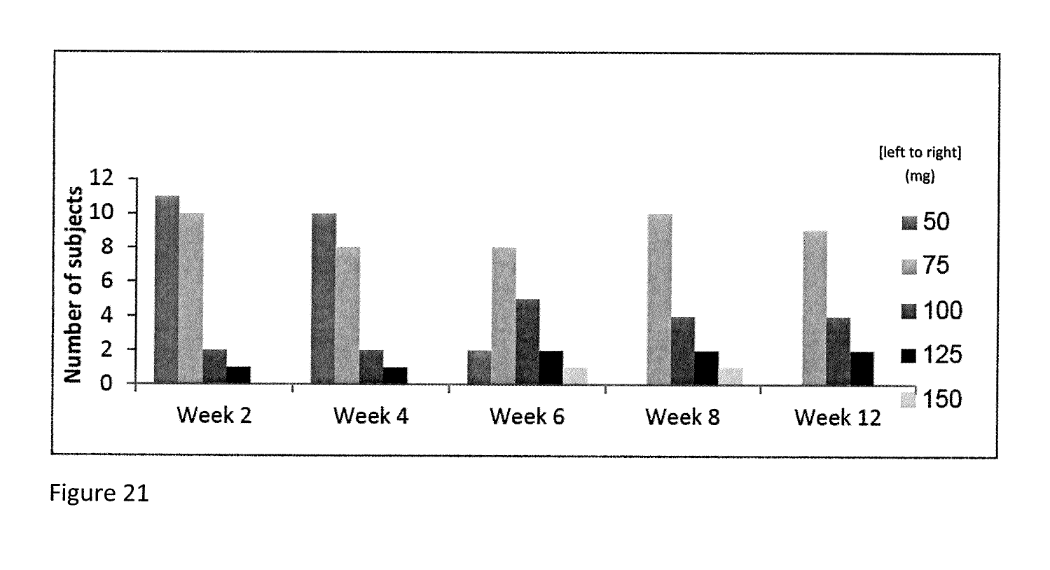

While the daily dose of DR-6MP remained the same, PURINETHOL® dose per patient changed, as displayed in FIG. 20 and FIG. 21, according to the decision of the Study Safety Physician following review of the subjects' lab data. In addition, PURINETHOL® is a systemically acting drug whereas DR-6MP is a locally acting drug. The extent of its absorption into the blood after 12 weeks of treatment is unknown yet, although PK data for the single dose 40 mg DR-6MP clearly demonstrated negligible systemic absorption. Therefore, a comparison of the extent of exposure between the two drugs is limited.

### Adverse Events

### Brief Summary of Adverse Events

A summary of the AEs that were reported during the study is presented in Table 38. A total of 92 AEs were reported in the DR-6MP treatment arm. Of these, 84 were treatment-emergent and 33 were considered as related to the study drug. In the PURINETHOL® treatment arm, a total of 91 AEs were reported, 85 of which were treatment-emergent and 31 were considered reasonably related to the study drug.

TABLE 38
Summary of AEs

| | DR-6MP 80 mg | | PURINETHOL ® | | |
|---|---|---|---|---|---|
| | (N = 40) | | (N = 24) | | |
| Summary Incidence of Adverse Events | N | (%) | N | (%) | P-value* |
| Adverse Events | 92 | | 91 | | |
| Subjects with at least one Adverse Event | 27 | (67.5) | 23 | (95.8) | 0.0079 |
| TEAEs | 84 | | 85 | | |
| Subjects with at least one TEAE | 27 | (67.5) | 23 | (95.8) | 0.0079 |
| Drug-related TEAEs | 33 | | 31 | | |
| Subjects with at least one drug-related TEAE | 11 | (27.5) | 11 | (45.8) | 0.1349 |
| SAES | 11 | | 2 | | |
| Subjects with at least one SAE | 8 | (20.0) | 2 | (8.3) | |
| Drug-related SAEs | 5 | | 1 | | |
| Subjects with at least one Drug-related SAE | 3 | (7.5) | 1 | (4.2) | 0.5938 |

SAE = serious adverse event,
TEAE = treatment-emergent adverse event
*P-value from Chi-Square test indicates the statistical significance of the difference between the treatments

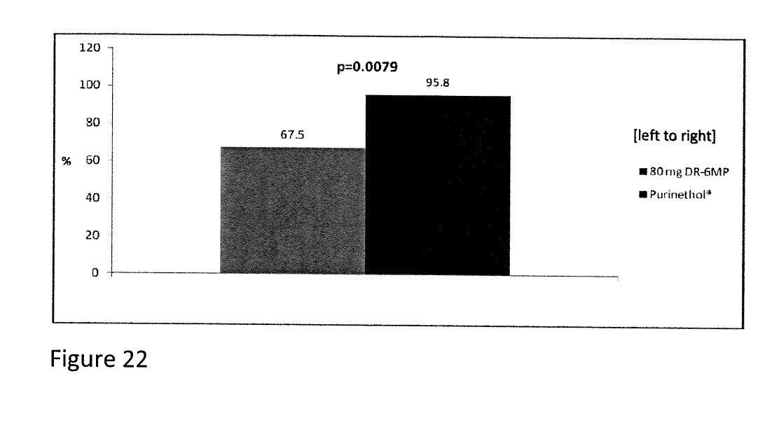

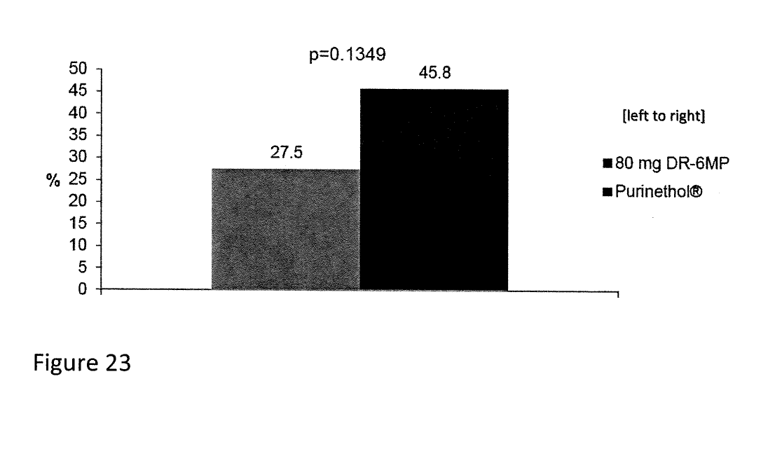

The majority of subjects treated with PURINETHOL® (95.8%) reported at least one AE compared with 67.5% of subjects treated with DR-6MP (p=0.0079; Table 38 and FIG. 22). Almost half of the subjects treated with PURINETHOL® (45.8%) reported at least one AE that was considered reasonably related to the study drug compared with 27.5% of subjects in the DR-6MP treatment arm (p=−0.1349, Table 38 and FIG. 23).

### Display of Treatment-Emergent Adverse Events

The incidence of treatment-emergent AEs (TEAEs) occurring in 5% or more patients is shown in Table 39. The incidence of drug-related TEAEs is displayed in Table 40.

TABLE 39
Summary of TEAEs occurring in
≧5% of patients in any treatment arm

| | DR-6MP | | | |
|---|---|---|---|---|
| | 80 mg | | PURINETHOL ® | |
| MedDRA System class/ | (N = 40) | | (N = 24) | |
| Preferred Term | N | (%) | N | (%) |
| Total treatment emergent AEs | 82 | | 85 | |
| Cardiac disorders | 0 | (0) | 2 | (8.3) |
| Gastrointestinal disorders | 17 | (42.5) | 13 | (54.2) |
| Abdominal pain | 4 | (10.0) | 5 | (20.8) |
| Abdominal pain upper | 1 | (2.5) | 4 | (16.7) |
| Constipation | 3 | (7.5) | 2 | (8.3) |
| Crohn's disease | 4 | (10.0) | 2 | (8.3) |
| Dyspepsia | 3 | (7.5) | 1 | (4.2) |
| Nausea | 7 | (17.5) | 6 | (25.0) |
| Pancreatitis acute/pancreatitis | 2 | (5.0) | 1 | (4.2) |
| Vomiting | 6 | (15.0) | 2 | (8.3) |
| General disorders and | 5 | (12.5) | 8 | (33.3) |
| administration site conditions | | | | |
| Asthenia | 1 | (2.5) | 5 | (20.8) |
| Pyrexia | 2 | (5.0) | 4 | (16.7) |
| Infections and infestations | 5 | (12.5) | 5 | (20.8) |
| Nasopharyngitis | 0 | (0) | 2 | (8.3) |
| Upper respiratory tract infection | 4 | (10.0) | 1 | (4.2) |
| Investigations | 2 | (5.0) | 3 | (12.5) |
| Metabolism and nutrition disorders | 1 | (2.5) | 4 | (16.7) |
| Decreased appetite | 1 | (2.5) | 4 | (16.7) |
| Musculoskeletal and connective | 5 | (12.5) | 4 | (16.7) |
| tissue disorders | | | | |
| Arthralgia | 3 | (7.5) | 2 | (8.3) |
| Nervous system disorders | 10 | (25.0) | 7 | (29.2) |
| Dizziness | 3 | (7.5) | 2 | (8.3) |
| Headache | 7 | (17.5) | 5 | (20.8) |
| Respiratory, thoracic and | 2 | (5.0) | 2 | (8.3) |
| mediastinal disorders | | | | |
| Skin and subcutaneous tissue | 3 | (7.5) | 1 | (4.2) |
| disorders | | | | |
| Rash | 2 | (5.0) | 0 | (0) |
| Vascular disorders | 3 | (7.5) | 0 | (0) |

TABLE 40
Summary of drug-related treatment-emergent AEs
occurring in ≧5% of patients in any treatment arm

| | DR-6MP 80 mg | | PURINETHOL ® | |
|---|---|---|---|---|
| MedDRA System class/ | (N = 40) | | (N = 24) | |
| Preferred Term | N | (%) | N | (%) |
| Drug-related treatment- | 35 | | 31 | |
| emergent AEs | | | | |
| Gastrointestinal disorders | 8 | (20.0) | 8 | (33.3) |
| Abdominal pain | 2 | (5.0) | 3 | (12.5) |
| Abdominal pain upper | 1 | (2.5) | 2 | (8.3) |
| Constipation | 2 | (5.0) | 1 | (4.2) |
| Nausea | 4 | (10.0) | 3 | (12.5) |
| Pancreatitis acute/ | 2 | (5.0) | 1 | (4.2) |
| pancreatitis | | | | |
| Vomiting | 2 | (5.0) | 1 | (4.2) |
| General disorders and | 2 | (5.0) | 2 | (8.3) |
| administration site | | | | |
| conditions | | | | |
| Asthenia | 0 | (0) | 2 | (8.3) |
| Infections and infestations | 2 | (5.0) | 1 | (4.2) |
| Investigations | 2 | (5.0) | 2 | (8.3) |
| Metabolism and nutrition | 1 | (2.5) | 3 | (12.5) |
| disorders | | | | |
| Decreased appetite | 1 | (2.5) | 3 | (12.5) |
| Nervous system disorders | 3 | (7.5) | 3 | (12.5) |
| Dizziness | 1 | (2.5) | 2 | (8.3) |
| Headache | 2 | (5.0) | 1 | (4.2) |

### Analysis of Adverse Events

Most AEs reported during the study were transient and of mild or moderate severity. Overall, statistically significantly fewer AEs were reported for the DR-6MP subjects (67.5%), relative to the PURINETHOL® subjects (95.8%, p=0.007⁹; FIG. 22). A higher proportion of subjects in the PURINETHOL® treatment arm (45.8%) reported drug-related AEs compared with subjects in the DR-6MP treatment arm (27.5%) although the difference between groups was not statistically significantly different (p=0.1349, FIG. 23).

The most common drug-related TEAEs in both treatment arms were GI disorders whose rate was higher in the PURINETHOL® treatment arm compared with that of the DR-6MP treatment arm (33.3% vs. 20%, respectively). The proportion of subjects with drug-related nausea, abdominal pain, decreased appetite, upper abdominal pain, asthenia and dizziness was higher in the PURINETHOL® treatment arm compared with the DR-6MP treatment arm (Table 40).

### Deaths, Other Serious Adverse Events, and Other Significant Adverse Events

### Listing of Deaths, Other Serious Adverse Events and Certain Other Significant Adverse Events

A total of 15 subjects reported SAEs during the entire reporting period (i.e., from signature of informed consent until 30 days after study completion). The events were all noted to be SAEs because patients were hospitalized. SAEs for 3 subjects occurred prior to study treatment, during the screening period and were not related to the study drug because the patients had not started treatment yet (Table 44); SAEs for 2 subjects occurred within 30 days after completion and were not considered related to the study drug (Table 44); 10 subjects reported SAEs that occurred during the 12-week treatment period (Table 41), of which 4 subjects had SAEs related to the study drug.

Table 41 presents a summary of the SAEs that were reported during the study.

TABLE 41
Summary of SAEs

| | 80 mg DR-6MP (N = 40) | | PURINETHOL ® (N = 24) | |
|---|---|---|---|---|
| System Organ Class/ | Subjects | Events | Subjects | Events |

| Preferred Term | N | (%) | N | (%) | N | (%) | N | (%) |
|---|---|---|---|---|---|---|---|---|
| Total SAEs | 11 | | | | 2 | | | |
| Blood and lymphatic | 0 | (0) | 0 | (0) | 1 | (4.2) | 1 | (50.0) |
| system disorders | | | | | | | | |
| Anaemia | 0 | (0) | 0 | (0) | 1 | (4.2) | 1 | (50.0) |
| Gastrointestinal disorders | 6 | (15.0) | 9 | (81.8) | 1 | (4.2) | 1 | (50.0) |
| Abdominal pain* | 2 | (5.0) | 2 | (18.2) | 0 | (0) | 0 | (0) |
| Crohn's disease | 2 | (5.0) | 2 | (18.2) | 1 | (4.2) | 1 | (50.0) |
| Intestinal obstruction | 1 | (2.5) | 1 | (9.1) | 0 | (0) | 0 | (0) |
| Nausea* | 1 | (2.5) | 1 | (9.1) | 0 | (0) | 0 | (0) |
| Pancreatitis acute* | 2 | (5.0) | 2 | (18.2) | 0 | (0) | 0 | (0) |
| Vomiting* | 1 | (2.5) | 1 | (9.1) | 0 | (0) | 0 | (0) |
| Infections and infestations | 1 | (2.5) | 1 | (9.1) | 0 | (0) | 0 | (0) |
| Anal abscess | 1 | (2.5) | 1 | (9.1) | 0 | (0) | 0 | (0) |
| Vascular disorders | 1 | (2.5) | 1 | (9.1) | 0 | (0) | 0 | (0) |
| Hypertension | 1 | (2.5) | 1 | (9.1) | 0 | (0) | 0 | (0) |

*These 4 events were all reported by one patient and are not separate AEs; rather, the abdominal pain, nausea and vomiting are manifestations of the acute pancreatitis.

Table 42 outlines the 13 SAEs reported in 10 subjects during the 12 week treatment period including drug relationship, event duration, treatment details and outcome.

TABLE 42
Serious adverse events

| | Preferred | System | SAE Duration | | AE related to | | Outcome |
|---|---|---|---|---|---|---|---|
| Treatment | Term | Organ Class | (days) | Severity | study drug? | Action taken | of Event |
| 80 mg DR-6MP | Abdominal | Gastrointestinal | 15 | Moderate | No reasonable | Medication taken + | Resolved |
| | pain | disorders | | | possibility | discontinued study + | |
| | | | | | | hospitalization | |
| 80 mg DR-6MP | Hypertension | Vascular | 4 | Mild | No reasonable | Medication taken + | Resolved |
| | | disorders | | | possibility | hospitalization | |
| 80 mg DR-6MP | Intestinal | Gastrointestinal | 93 | Severe | No reasonable | Medication taken + | Resolved |
| | obstruction | disorders | | | possibility | discontinued study + | |
| | | | | | | hospitalization | |
| 80 mg DR-6MP | Crohn's disease | Gastrointestinal | 3 | Severe | No reasonable | Medication taken + | Resolved |
| | | disorders | | | possibility | discontinued study + | |
| | | | | | | hospitalization | |
| 80 mg DR-6MP | Crohn's disease | Gastrointestinal | 13 | Moderate | No reasonable | Medication taken + | Resolved |
| | | disorders | | | possibility | discontinued study + | |
| | | | | | | hospitalization | |
| 80 mg DR-6MP | Pancreatitis | Gastrointestinal | 4 | Moderate | Reasonable | Medication taken + | Resolved |
| | acute | disorders | | | possibility | discontinued study + | |
| | | | | | | hospitalization | |
| 80 mg DR-6MP | Anal abscess | Infections and | 18 | Severe | Reasonable | Medication taken + | Resolved |
| | | infestations | | | possibility | discontinued study + | |
| | | | | | | hospitalization | |
| 80 mg DR-6MP | Nausea* | Gastrointestinal | 7 | Severe | Reasonable | Hospitalization | Resolved |
| | | disorders | | | possibility | | |
| | Abdominal | Gastrointestinal | 7 | Severe | Reasonable | Hospitalization | Resolved |
| | pain* | disorders | | | possibility | | |
| | Vomiting* | Gastrointestinal | 7 | Severe | Reasonable | Hospitalization | Resolved |
| | | disorders | | | possibility | | |
| | Pancreatitis | Gastrointestinal | 107 | Severe | Reasonable | Medication taken + | Resolved |
| | acute* | disorders | | | possibility | discontinued study + | |
| | | | | | | hospitalization | |
| PURINETHOL ® | Anemia | Blood and | 2 | Moderate | Reasonable | Medication taken + | Improved |
| | | lymphatic | | | possibility | discontinued study + | |
| | | system disorders | | | | hospitalization | |
| PURINETHOL ® | Crohn's | Gastrointestinal | 6 | Severe | No reasonable | Discontinued study + | Resolved |
| | disease | disorders | | | possibility | hospitalization | |

Table 43 summarizes the SAEs that occurred during the screening process. Three SAEs were reported in 3 subjects. None of these subjects were treated with study drugs, although 2 of the subjects had already been randomized to a treatment arm.

TABLE 43
Summary of SAEs in Screening Failures and Pre-baseline events

| Treatment | PT | SOC | Duration of SAE (days) | Intensity/severity |
|---|---|---|---|---|
| — | Abdominal | Gastrointestinal | 3 | Mild |
| | Pain | disorders | | |
| 80 mg DR-6MP | Pyrexia | General disorders and | 4 | Severe |
| | | administration site | | |
| | | conditions | | |
| PURINETHOL ® | Abdominal | Gastrointestinal | 4 | Moderate |
| | Pain | disorders | | |

Table 44 summarizes the SAEs that occurred within 30 days of study completion. Two subjects reported 2 SAEs which occurred after study completion and within 30 days of termination and were considered not related to the study drug.

TABLE 44
Summary of SAEs in Screening Failures and Pre-baseline events

| | | | Duration of | Intensity/ |
|---|---|---|---|---|
| Treatment | PT | SOC | SAE (days) | severity |
| 80 mg DR- | Abdominal pain; | Gastrointestinal | 2 | Mild |
| 6MP | vomiting | disorders | | |
| 80 mg DR- | CD exacerbation | Gastrointestinal | 8 | Severe |
| 6MP | | disorders | | |

### Analysis and Discussion of Deaths, Other Serious Adverse Events and Certain Other Significant Adverse Events

A total of 13 SAEs were reported by 10 subjects during the study. Most SAEs reported were transient and resolved within a few days. Eleven SAEs were reported by 8 subjects (20.0%) in the DR-6MP treatment arm, of which 6 reported by 3 subjects (7.5%) were considered to be drug-related. Specifically, 4 of these drug-related SAEs (nausea, abdominal pain, vomiting and acute pancreatitis) occurred in one subject; one subject reported acute pancreatitis and one subject reported an anal abscess. All SAEs resolved following hospitalization and administration of medication. In the PURINETHOL® treatment arm, 2 subjects (8.2%) each reported one SAE: a moderate drug-related SAE (anemia), which was improved but not resolved at study end and a severe unrelated SAE (CD exacerbation) which resolved.

### Clinical Laboratory Evaluation

### Evaluation of Each Laboratory Parameter

The most important safety issues related to thiopurine use relate to the known increase of leucopenia (evidenced by a marked reduction in WBC count), hepatotoxicity (evidenced by a marked increase in LFTs, ALT, AST and bilirubin) and pancreatitis (generally associated with elevations in amylase with accompanying abdominal pain, nausea and vomiting) following 6MP or azathioprine treatment. Therefore, in addition to standard AE reporting, these 3 events were carefully monitored during the study, with laboratory testing of WBC, LFTs and amylase at each bi-weekly clinic visit as well as at a special laboratory safety evaluation visit one week following baseline. A review of the laboratory findings related to these 3 important safety issues is described below.

### Leucopenia and Hematology Test Values

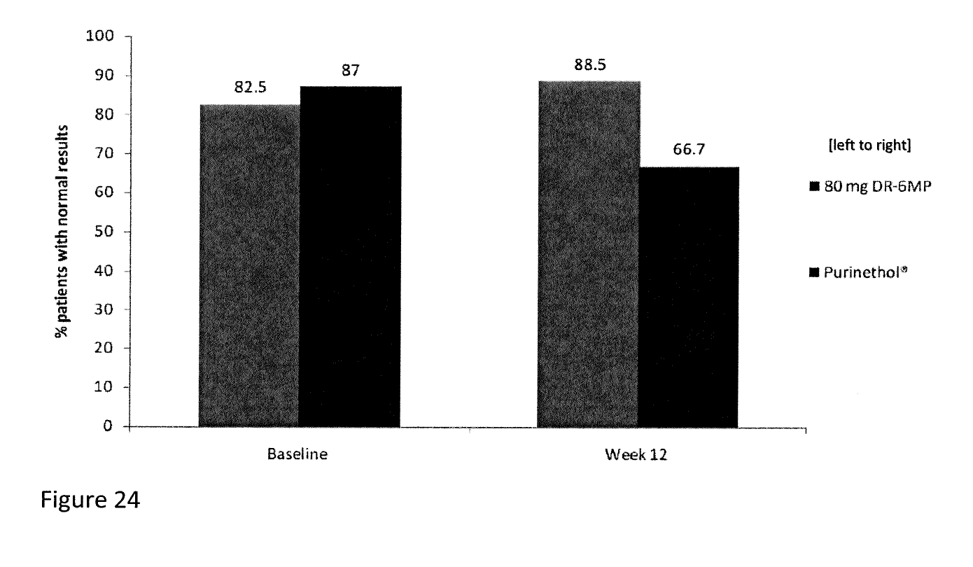

There was no evidence of leucopenia in the DR-6MP group after 12 weeks of treatment. The percent of subjects in the DR-6MP treatment arm who had WBC within the normal range after 12 weeks of treatment was comparable and even higher than the percent of subjects at baseline, whereas for the PURINETHOL® treatment arm, there was a greater than 20% drop in the percent of subjects with WBC in the normal range at week 12, relative to baseline. The percent of subjects with WBC within normal range at week 12 in the DR-6MP treatment arm was 88.5% compared with only 66.7% in the PURINETHOL® group (FIG. 24).

A review of the actual mean hematology test values indicates that there was a continuous decrease in WBC for both treatments, with a statistically significant change from baseline apparent in the DR-6MP treatment only at week 12, whereas there were statistically significant decreases in the PURINETHOL® treatment arm beginning as early as week 4 onward (Table 45).

TABLE 45
Values and Changes from baseline in WBC by treatment (ITT population)

| | WBC | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| baseline | 40 | 7.75 ± 2.29 | | 23 | 8.27 ± 2.31 | | |
| Change at week 1 | 37 | −0.15 ± 2.06 | 0.651 | 21 | −0.60 ± 2.56 | 0.299 | 0.4756 |
| Change at week 2 | 33 | −0.16 ± 1.77 | 0.615 | 20 | −0.48 ± 2.28 | 0.354 | 0.5603 |
| Change at week 4 | 30 | −0.48 ± 1.96 | 0.189 | 18 | −1.48 ± 2.46 | 0.021 | 0.1279 |
| Change at week 6 | 29 | −0.21 ± 2.16 | 0.606 | 16 | −2.08 ± 2.48 | 0.004 | 0.0118 |
| Change at week 8 | 28 | −0.50 ± 1.78 | 0.153 | 14 | −2.25 ± 2.03 | 0.001 | 0.0065 |
| Change at week 12 | 27 | −0.79 ± 1.92 | 0.042 | 11 | −1.62 ± 2.17 | 0.033 | 0.2539 |

SD = standard deviation,
WBC = white blood cells
*P value by T test for the statistical significance of WBC change within treatment;
**P value by T test for the statistical significance of the difference in WBC change between treatments

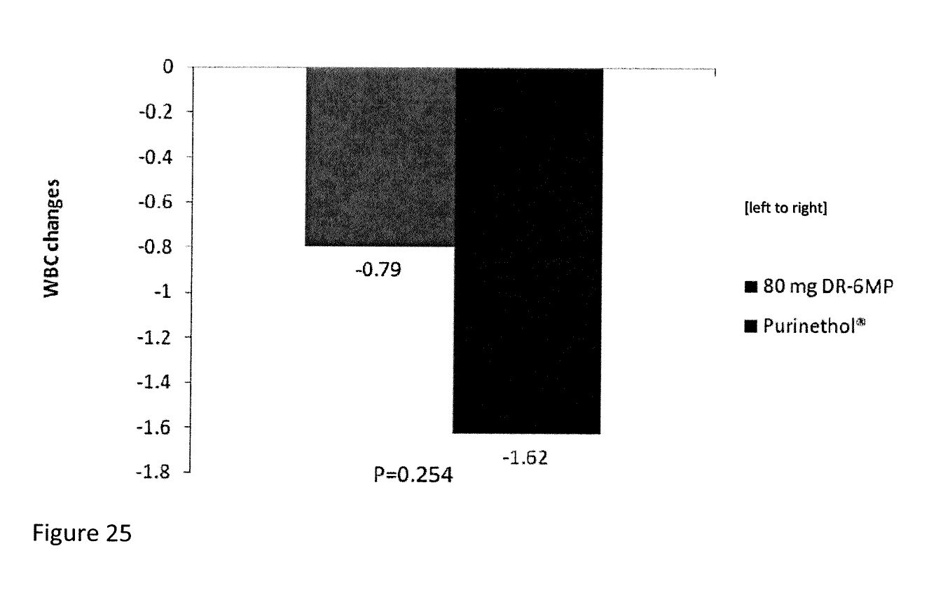

At the conclusion of the study, following 12 weeks of treatment, subjects in the PURINETHOL® group showed a greater mean decrease in WBC relative to baseline compared to the DR-6MP treatment, although the difference between treatments was not statistically significant (FIG. 25). It is interesting to note, however, that at weeks 6 and 8, when the PURINETHOL® dose was titrated up to maximum therapeutic levels, the mean decreases in WBC relative to baseline for the PURINETHOL® group were at their highest, and the differences between treatments were statistically significant.

An additional analysis was done of the WBC change within each treatment group by matching subjects from the DR-6MP group and from the PURINETHOL® group by baseline weight and then assessing the WBC change from baseline for each treatment. It was presumed that this, albeit artificial but select data set, represents the most closely matched subjects each receiving the same thiopurine with one group receiving a fixed dose of 80 mg DR-6MP, presumably locally delivered, and with the other group receiving 1-1.5 mg/kg PURINETHOL® (between 50-150 mg) systemically delivered. An evaluation of this data set would provide both (a) the safety profile of each treatment as it impacts on changing WBC levels and (b) indirect evidence of how the drug is delivered (systemically or locally).

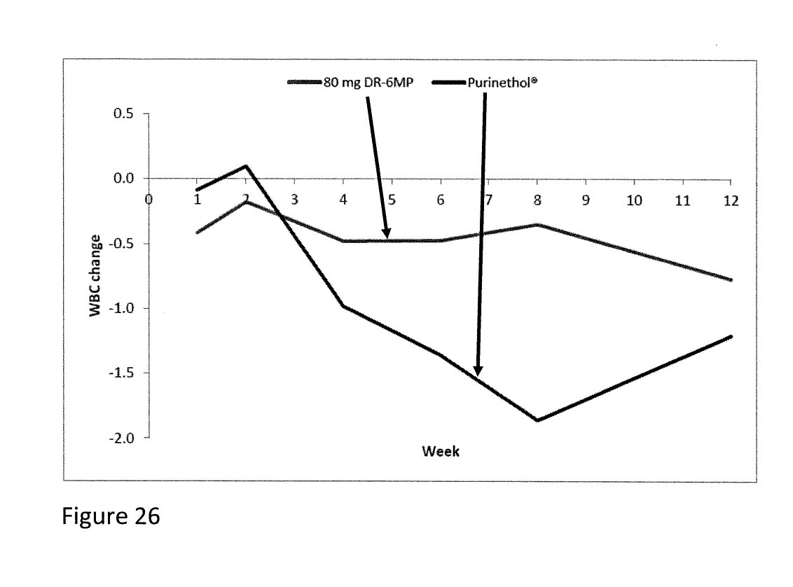

As can be seen in FIG. 26, the effect on the WBC for the DR-6MP subgroup is consistent and steady, and unlike the PURINETHOL® treatment, does not really change substantially over time, implying that it is unlikely that the DR-6MP is systemically absorbed. PURINETHOL® treatment, on the other hand, demonstrates substantive changes over time, underscoring both that cumulative dosing is necessary for both the clinical efficacy and potential toxic effect of this systemically absorbed drug, and that the WBC decreases and increases are dose dependent, subject to titration increases to maximum therapeutic dose, as well as subsequent necessary reductions in dose. Furthermore, the WBC decrease observed for the DR-6MP group is far less than that seen for the PURINETHOL® treatment group, suggesting that the potential for leucopenia is greater for PURINETHOL®.

### Hepatotoxicity and Chemistry Test Values

There were fewer events of drug-induced hepatotoxicity in the DR-6MP treatment arm compared to the PURINETHOL® arm. Three events of elevated LFTs necessitating treatment discontinuation were reported: one patient in the DR-6MP treatment arm (2.5%) and 2 patients in the PURINETHOL® treatment arm (8.3%) had to permanently discontinue the study due to hepatotoxicity.

In the DR-6MP treatment arm, the elevation in LFTs would not have mandated study termination but only drug dose modification; however since that option was not possible in the DR-6MP treatment arm, the subject had to be terminated from the study.

In the PURINETHOL® treatment arm, two subjects had increased LFTs (ALT, AST and bilirubin) considered to be related to the study drug; values for one of whom were a “red alert” according to the study protocol necessitating immediate drug termination.

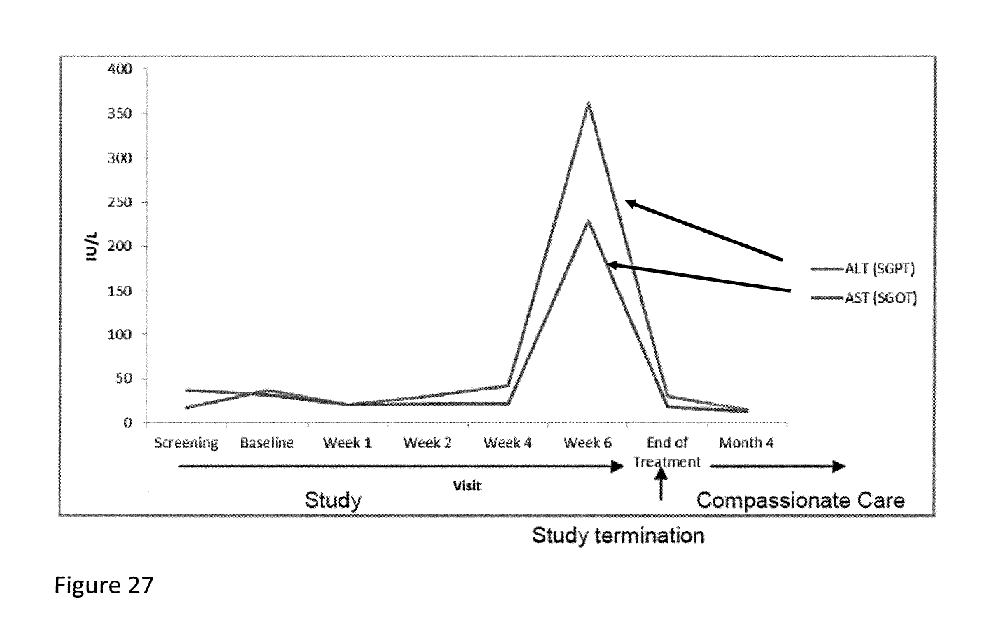

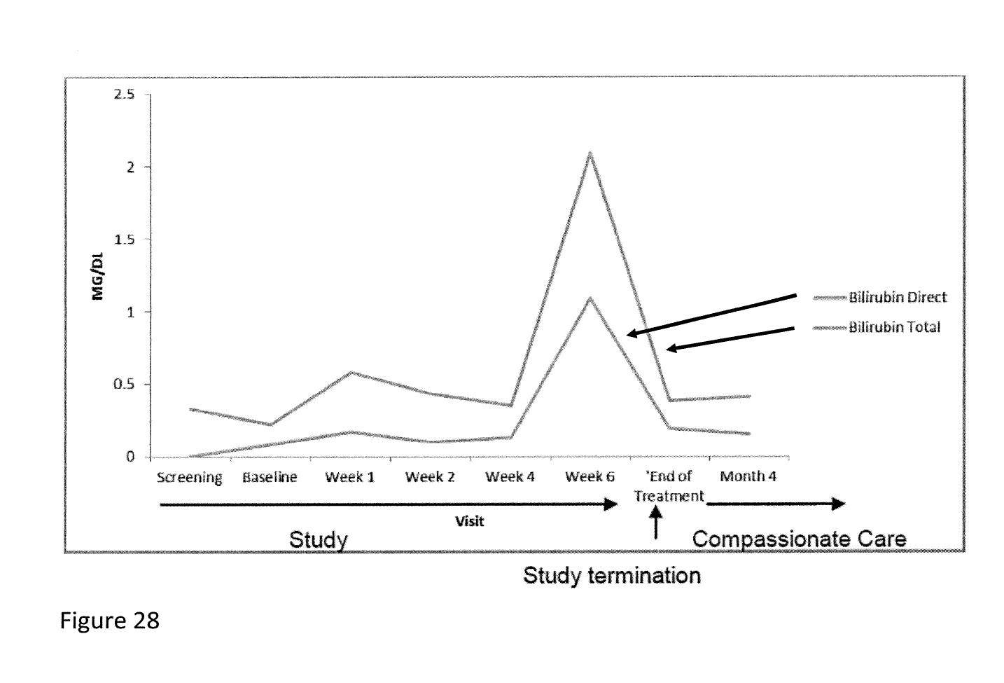

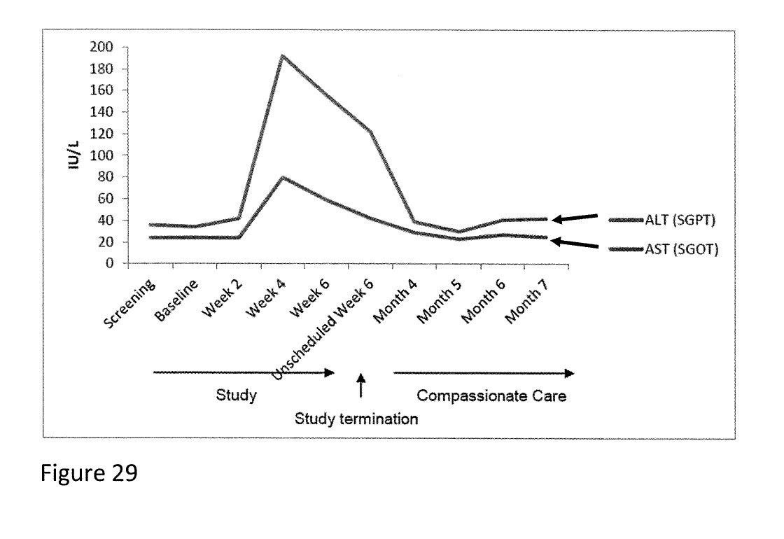

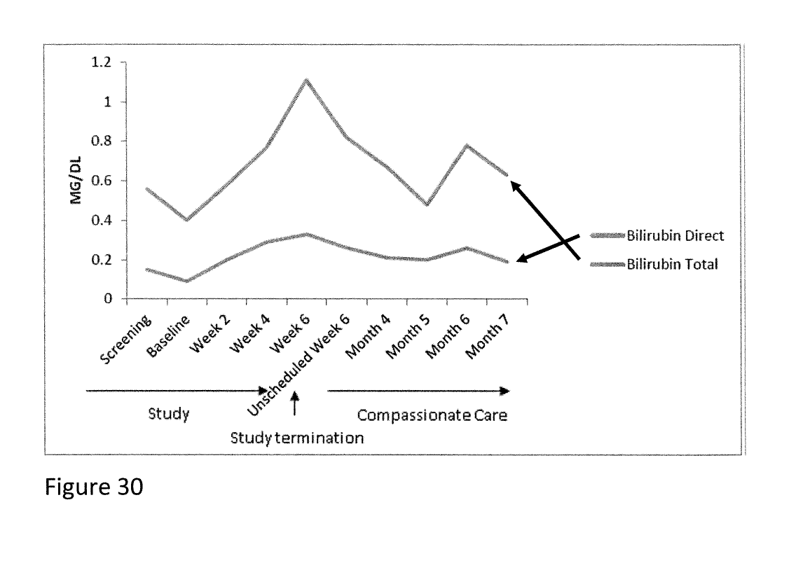

Following treatment termination, however, both subjects received DR-6MP treatment on a “compassionate care” basis subsequent to petitioning of their site PIs and approval of their respective IRBs. In both cases, the subjects' LFTs stabilized on DR-6MP and remained within normal limits for the period of “compassionate care” for several months as depicted in FIGS. 27-30.

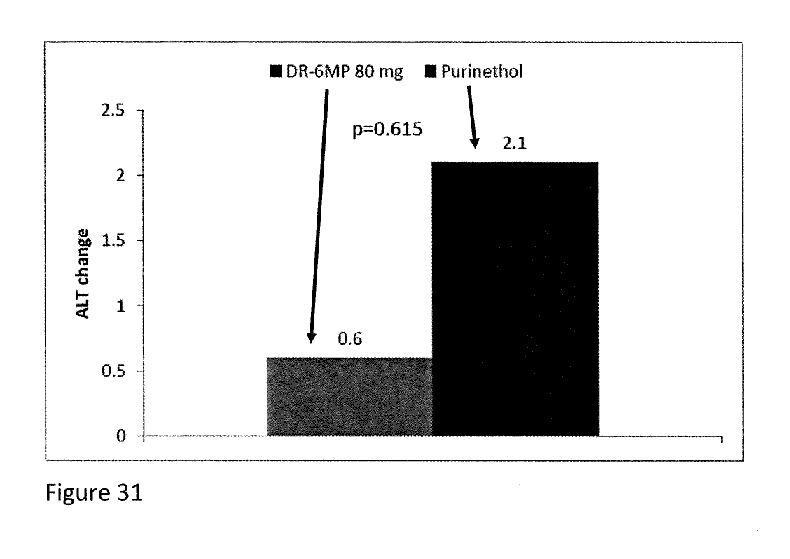

A review of the actual mean chemistry laboratory test values indicates that other than a significant decrease in AST levels observed between baseline and visit 2 in the PURINETHOL® treatment arm (p=0.043, Table 46), and a significant increase in ALT between baseline and week 4 in the DR-6MP treatment arm (p=0.046, Table 47), no significant changes in AST or ALT levels from baseline were observed either within each treatment arm or between treatment arms. The increase in ALT at week 12, relative to baseline, was greater for the PURINETHOL® group, compared to the DR-6MP group, although the difference was not statistically significant (Table 47 and FIG. 31).

TABLE 46
Values and Changes from baseline in AST by treatment (ITT population)

| | AST | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| baseline | 40 | 18.7 ± 6.3 | | 23 | 19.3 ± 5.1 | | |
| Change at week 1 | 37 | 0.0 ± 3.6 | 1.000 | 21 | −0.8 ± 5.2 | 0.508 | 0.5534 |
| Change at week 2 | 33 | −1.0 ± 4.1 | 0.179 | 20 | −2.0 ± 4.0 | 0.043 | 0.3963 |
| Change at week 4 | 30 | 1.1 ± 5.9 | 0.319 | 18 | 2.1 ± 14.0 | 0.532 | 0.7745 |
| Change at week 6 | 29 | −0.7 ± 6.8 | 0.581 | 16 | 14.2 ± 49.7 | 0.272 | 0.2514 |
| Change at week 8 | 28 | 0.2 ± 5.4 | 0.862 | 14 | −1.6 ± 4.1 | 0.158 | 0.2736 |
| Change at week 12 | 27 | −0.2 ± 6.1 | 0.852 | 11 | −0.2 ± 5.6 | 0.916 | 0.9850 |

SD = standard deviation,
AST = aspartate aminotransferase
*P value by T test for the statistical significance of AST change within treatment;
**P value by T test for the statistical significance of the difference in AST change between treatments

TABLE 47
Values and Changes from baseline in ALT by treatment (ITT population)

| | ALT | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| baseline | 40 | 18.0 ± 12.2 | | 23 | 16.9 ± 7.3 | | |
| Change at week 1 | 37 | 0.9 ± 5.1 | 0.298 | 21 | 0.4 ± 6.1 | 0.779 | 0.7359 |
| Change at week 2 | 33 | 0.5 ± 5.6 | 0.580 | 20 | 0.7 ± 4.5 | 0.524 | 0.9439 |
| Change at week 4 | 30 | 3.3 ± 8.8 | 0.046 | 18 | 11.9 ± 37.0 | 0.191 | 0.3476 |
| Change at week 6 | 28 | 2.1 ± 11.4 | 0.330 | 16 | 28.9 ± 84.5 | 0.191 | 0.2261 |
| Change at week 8 | 27 | 1.7 ± 7.6 | 0.263 | 14 | 1.5 ± 4.1 | 0.189 | 0.9273 |
| Change at week 12 | 27 | 0.6 ± 9.0 | 0.734 | 11 | 2.1 ± 5.9 | 0.269 | 0.6145 |

SD = standard deviation,
ALT = alanine amimotransferase
*P value by T test for the statistical significance of ALT change within treatment;
**P value by T test for the statistical significance of the difference in ALT change between treatments

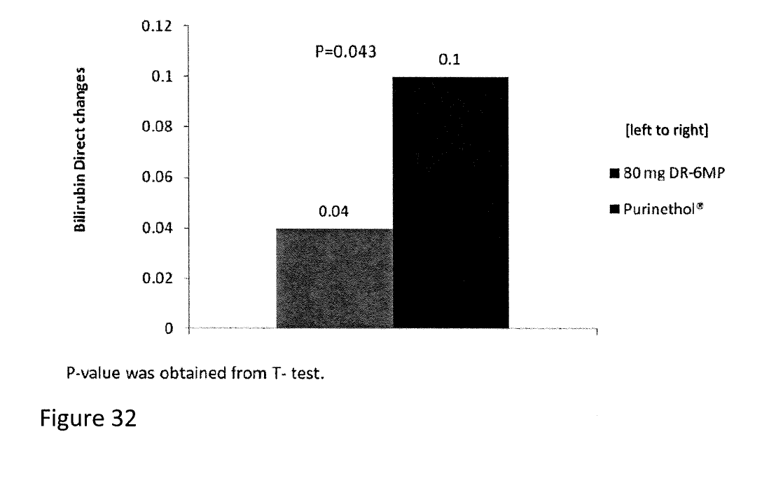

In the DR-6MP treatment arm, a significant increase from baseline in direct bilirubin was observed on week 2, 6 and 12, In the PURINETHOL® treatment arm, a significant increase from baseline was observed on weeks 1, 4 and 12. The extent of increase in direct bilirubin levels from baseline was similar in both treatment arms on all visits except for week 1 and week 12 during which a statistically significant difference between the DR-6MP and the PURINETHOL® treatment arms was observed (p=0.0237 and p=0.0425, respectively; Table 48 and FIG. 32).

TABLE 48
Values and Changes from baseline in bilirubin direct by treatment (ITT population)

| | Bilirubin direct | | | | | | |
|---|---|---|---|---|---|---|---|
| | DR-6MP 80 mg | | | PURINETHOL ® | | | |
| | N | Mean ± SD | P value* | N | Mean ± SD | P value* | P value** |
| baseline | 40 | 0.13 ± 0.09 | | 23 | 0.12 ± 0.08 | | |
| Change at week 1 | 37 | 0.01 ± 0.08 | 0.424 | 21 | 0.06 ± 0.09 | 0.004 | 0.0237 |
| Change at week 2 | 33 | 0.04 ± 0.09 | 0.033 | 20 | 0.04 ± 0.10 | 0.104 | 0.9576 |
| Change at week 4 | 30 | 0.03 ± 0.09 | 0.150 | 18 | 0.05 ± 0.09 | 0.052 | 0.4504 |
| Change at week 6 | 28 | 0.04 ± 0.09 | 0.028 | 16 | 0.10 ± 0.26 | 0.155 | 0.3823 |
| Change at week 8 | 28 | 0.03 ± 0.09 | 0.151 | 14 | 0.06 ± 0.11 | 0.066 | 0.3102 |
| Change at week 12 | 27 | 0.04 ± 0.07 | 0.005 | 11 | 0.10 ± 0.08 | 0.002 | 0.0425 |

SD = standard deviation,
*P value by T test for the statistical significance of bilirubin if red change within treatment;
**P value by T test for the statistical significance of the difference in bilirubin direct change between treatments

### Pancreatitis and Amylase

During the study, there were no significant changes from baseline of amylase either within each treatment arm or between treatment arms. However, individual incidents of pancreatitis with elevated amylase levels were reported. Two events of pancreatitis were reported in 2 patients (5%) in the DR-6MP treatment arm and one event of pancreatitis was reported in one patient (4.2%) in the PURINETHOL® treatment arm as described below.

Two SAEs of acute pancreatitis that required hospitalization occurred in the DR-6MP group. The first case occurred in a subject who entered the study with a history of pancreatitis, and was therefore, put on DR-6MP as a “thiopurine failure”. Furthermore, this subject entered the study with elevated amylase levels of 139 that increased at week 1 to 187 and was 349 upon termination from the study. According to the Study Safety Physician, this event of pancreatitis could have been disease related and the subject most probably should not have been included in the study due to the elevated amylase levels at entry. The second subject entered the study with an amylase of 90. At week 1, the amylase increased to 522 which most likely indicates drug-related pancreatitis. The subject was hospitalized and treated and prematurely terminated from the study.

One case of pancreatitis that was considered to be drug-related was reported in a subject in the PURINETHOL® group. The subject entered the study with amylase of 64. At week 4, amylase levels increased to 108 and were 143 upon termination from the study. This AE did not result in hospitalization but the subject was discontinued from the study.

In summary, pancreatitis can be either the result of a drug reaction or related to the underlying disease. Three events of pancreatitis, with elevated amylase levels were reported in the study with comparable rates for the two treatment arms. In the DR-6MP group, the 2 events required hospitalization and were therefore reported as SAEs, while for the PURINETHOL® group, no hospitalization was required. All subjects prematurely terminated.

### Vital Signs, Physical Findings and Other Observations Related to Safety

The routine clinic visit included a physical examination and vital signs (blood pressure, pulse, oral temperature, weight). Height was also measured at the screening visit to determine BMI and the standard weight required for CDAI calculations. An ECG was done at screening, baseline and week 12.

A few clinically significant physical findings were reported for 1 patient (2.5%) in the DR-6MP arm and for 3 patients (12.5%) in the PURINETHOL® treatment arm, but were not considered to be drug or study related. No clinically significant changes in vital signs or ECG were recorded during the study. However, an interesting finding demonstrating positive weight increases for the DR-6MP arm relative to the PURINETHOL® arm was noted during the study. As detailed above in the clinical efficacy section, weight and BMI increased in the DR-6MP treatment arm during the 12 weeks of treatment while both parameters decreased in the PURINETHOL® treatment arm. A comparison between the treatment arms of the change in weight and BMI between baseline and each study visit, showed a statistically significant difference between the two treatment arms at visit 8 (p=0.0121 for both parameters by median test). Although this parameter is typically a marker for safety, it also demonstrates the clinical efficacy of the DR-6MP.

### Unscheduled Visits

According to the protocol, following screening and baseline visits, subjects were to be seen for clinical evaluation and laboratory testing every two weeks from week 2 through week 8, with an additional safety laboratory visit at week 1 and a final termination visit at week 12. Additionally, “unscheduled visits” could be performed at any time during the study at the subject's request or as deemed necessary by the investigator. A smaller proportion of patients in the DR-6MP treatment arm (12.5%) had unscheduled visits due to AEs, compared with those in the PURINETHOL® treatment arm.

### Premature Terminations

The proportion of patients who discontinued the study due to AEs was higher in the PURINETHOL® treatment arm compared with the DR-6MP arm (29.2% vs. 25%).

### Safety Conclusions

Overall, there was a statistically significantly higher proportion of AEs in the PURINETHOL® treatment arm compared with the DR-6MP treatment arm. The proportion of subjects with drug-related nausea, abdominal pain, decreased appetite, upper abdominal pain, asthenia and dizziness was higher in the PURINETHOL® treatment arm compared with the DR-6MP treatment arm. Most AEs reported during the study were transient and of mild or moderate severity.

The proportion of subjects who withdrew from the study due to AEs was similar in both treatment groups, but was slightly higher in the PURINETHOL® treatment arm. The proportion of subjects who withdrew from the study due to drug-related SAEs was higher in the DR-6MP treatment arm. Most SAEs reported were transient and resolved within a few days.

There was no evidence of drug-induced leucopenia in the DR-6MP group after 12 weeks of treatment. The percent of patients who had WBC within the normal range after 12 weeks of treatment was higher in the DR-6MP treatment arm (88.5%) compared with only 66.7% in the PURINETHOL® group. Patients in the PURINETHOL® group had a continuous decrease in their WBC during the trial with a statistically significant decrease from baseline, from week 4 onwards, while in the DR-6MP group, a statistically significant decrease in WBC levels from baseline was only observed between baseline and week 12. This data supports the notion that the drug is not absorbed.

The proportion of subjects who developed drug-induced hepatotoxicity was lower for DR-6MP than for PURINETHOL® (2.5% vs. 8.3%). The increase in mean ALT at week 12, relative to baseline, was greater for the PURINETHOL® group, compared to the DR-6MP group. Bilirubin direct significantly increased in the PURINETHOL® treatment arm at week 12 compared with the DR-6MP treatment arm. The two patients from the PURINETHOL® treatment arm who withdrew from the study prematurely due to hepatotoxicity, received DR-6MP on a “compassionate care basis”. While on DR-6MP, the previously elevated liver function tests returned to normal levels and were maintained at those levels for the period of observation—up to 7 months.

A comparable percentage of pancreatitis events occurred in both treatment arms (5% in the DR-6MP arm vs. 4.2% in the PURINETHOL® arm). This data supports the notion that DR-6MP is biologically active systemically, even though it is negligibly absorbed. Unlike hepatotoxicity or leucopenia which is dose-dependent, pancreatitis can be an idiosyncratic allergic reaction to even minute amounts of drug in susceptible patients.

No significant changes from baseline—either within each treatment arm or between treatment arms—were observed for vital signs, physical exam or ECG. As noted in the efficacy section, a comparison between the treatment arms of the change in median weight and BMI between baseline and each study visit showed a statistically significant difference between the two treatment arms at visit 8, as well as continued weight gain in the DR-6MP treatment arm, compared with continued weight loss in the PURINETHOL® treatment arm, throughout the study. Although weight change is typically a marker for safety, the prevention of weight loss that was seen in CD patients on DR-6MP demonstrates the drug's ability to modify the course of the disease.

In conclusion, DR-6MP was safer and better tolerated than PURINETHOL®.

### DISCUSSION AND OVERALL CONCLUSIONS

CD is a chronic inflammatory disorder that follows a progressive and destructive course. Ultimately, uncontrolled inflammation leads to bowel damage from disease-related complications such as strictures, fistulas and abscesses requiring surgical resection. Overall, CD patients have a poor outcome; regardless of the present medical therapy offered to them, development of complications occurs in 3 out of 4 patients in their lifetime. AZA and 6-MP have been suggested as a steroid-sparing, long-term treatment for patients with chronic active disease who have a severe flare requiring steroids or multiple steroid treatments during a year. Although these drugs are well established as steroid-sparing induction and maintenance agents, they are associated with myelosupression and hepatotoxicity, often requiring treatment discontinuation.

CD results from the breakdown of systemic immune tolerance towards intestinal-related antigens. Oral immune modulation is a new platform for therapy of immune-mediated disorders in which inflammation can be reduced without suppressing the systemic immune system. This method/paradigm is an active process which uses the unique inherent ability of the gastrointestinal immune system to control and suppress unwanted systemic immune responses by modifying different parts of the systemic immune system in an antigen-specific manner, thereby altering specific subsets of cells [40-44]. In contrast to oral tolerance techniques which involve the oral administration of disease-associated antigens and which were unsuccessful in most clinical trials over the past two decades, the oral administration of low levels of a non-absorbable immune modulator can alter the systemic immune system without suppressing it.

Targeted ileal delivery is appropriate in CD to modulate an effect in the intestinal immune system that can alter the systemic anti-inflammatory immune system with the potential for maximum clinical effect while reducing the disabling side effects of standard systemic therapies.

At 12 weeks, treatment with DR-6MP demonstrated non-inferiority compared with the reference treatment. The CDAI score was decreased across time (from baseline to week 12) in both treatment arms to a comparable extent, with a similar proportion of subjects in the DR-6MP and PURINETHOL® treatment arms exhibiting clinical response (remission or response) at week 12, demonstrating that a non-absorbable 6MP formulation, targeted locally, can achieve the same clinical effect as systemically delivered PURINETHOL®.

However, DR-6MP induced faster clinical response compared to the reference drug. A greater proportion of subjects achieved response at week 8 or attained remission at week 8 in the DR-6MP treatment arm, compared with the PURINETHOL® treatment arm. Furthermore, from week 6 onwards, a statistically significantly higher proportion of patients in the DR-6MP 80 treatment arm maintained remission for two consecutive visits compared with those in the PURINETHOL® treatment arm.

Therefore, the results show that DR-6MP treatment shows efficacy faster (a full 4 weeks earlier) than PURINETHOL®, and that by week 12, the PURINETHOL® “catches up”, as expected. Other studies have also shown an earlier effect; in the National Cooperative CD Study, the maximum decrease in the mean CDAI score for the azathioprine-treated patients was at 9 weeks [Summers et al. 1979]. In another study, azathioprine combined with steroids (tapered to 10 mg/day over 6 weeks) showed a significant benefit for azathioprine by week 8 [Ewe et al. 1993]. Sandbom et al. [2002] suggest that azathioprine or 6MP may act more rapidly than previously believed, perhaps over 4-8 weeks.

To correlate the clinical efficacy finding of CDAI, quality of life was evaluated using the IBDQ. Both treatment arms showed an improvement in quality of life after 12 weeks that was correlated with the reduction in CDAI. However, the improvement in quality of life at week 12 for the DR-6MP treatment arm was correlated with the reduction in CDAI score, already demonstrated at week 8. Furthermore, the improvement in quality-of-life assessment was observed in both responders and non-responders in the DR-6MP treatment arm, but not in the PURINETHOL® treatment arm.

Several subsets of patients were allowed to enroll into the study. The first subset, patients with an experience of previous thiopurine failure (either due to lack of clinical benefit or occurrence of non-serious adverse events), were included in the study but were only allocated to the DR-6MP treatment group in an effort to evaluate whether they may benefit from DR-6MP treatment despite their previous thiopurine failure. Indeed, these patients' CDAI score improved significantly between baseline and week 12, indicating that DR-6MP may be used in patients with a history of previous thiopurine failure, providing an additional option for treatment.

The second and third subsets of subjects analysed were those who were treated concomitantly with another drug for CD: 5-ASA or steroids/antibiotics. The subset of patients treated by 5-ASA concomitantly with DR-6MP showed a statistically significant improvement in CDAI scores at week 12 relative to baseline, while only a trend for improvement was shown for subjects treated concomitantly with PURINETHOL® and 5-ASA. Patients treated with DR-6MP concomitantly with low-dose steroids/antibiotics showed a statistically significant improvement from baseline in CDAI scores after 12 weeks of treatment, while the same subset of patients who were treated with PURINETHOL® did not show such improvement. It can therefore be concluded that combined treatment with DR-6MP can lead to improvement in patients who were not able to achieve a clinical response while on monotherapy with other CD treatments.

DR-6MP is delivered locally and is not absorbed systemically. Nevertheless, it seems to exert a systemic immunological effect comparable to that seen for the systemically delivered PURINETHOL®. Treatment with locally delivered DR-6MP resulted in a reduction in the general immunological systemic parameters: CRP and ESR. Although the reduction in CRP and ESR levels following DR-6MP treatment was somewhat less than that seen for PURINETHOL®, the fact that a non-absorbable locally delivered drug results in comparable reduction of systemic inflammatory parameters, as that following treatment with a systemically delivered drug, is significant.

Treatment with DR-6MP resulted in a decrease in IFN-gamma-secreting T cell clones in response to patient-derived bowel proteins, whereas PURINETHOL® led to an increase in IFN-gamma-secreting clones. In CD, the aberrant immune response is considered to be regulated by Type 1 T helper cells, resulting from the proliferation and differentiation of T cells into effector T cells, producing cytokines (such as interferon IFN-γ, interleukin IL-2, and IL-18) that magnify the immune response. Due to defective immune homeostasis in these patients, the reaction of the immune response does not terminate, and results in an ongoing exaggerated T-cell response. Hence, another index of clinical improvement in CD is modification of the circulating blood levels of systemic immunologic cells, including serum and intracellular cytokines, T cell subsets and specifically, those secreting pro-inflammatory cytokines such as IFN-γ. The reduction in IFN-γ levels in patients treated with DR-6MP implies improvement in the CD patient's immunological status.

DR-6MP led to a decrease of CD62 (adhesive proteins/selectins) expression on peripheral T lymphocytes as measured by FACS analysis, implying a reduction of lymphocyte adhesion to the site of inflammation and indicating improved immune response. In contrast, PURINETHOL® led to an increase in CD62+ expression. Immunohistochemistry studies of surgically resected specimens from patients with CD or ulcerative colitis have demonstrated that there is a statistically significant, nearly 4-fold increase in P-selectin immunoreactivity in the veins, venules and capillaries of highly inflamed gut compared to normal gut. This marked up-regulation of P-selectin in inflamed tissues adversely affects normal lymphocyte localization and recirculation, interfering with normal immune response. The reduction in CD62 adhesion protein, however, attests to the potential of the DR-6MP drug to decrease erratic leukocyte migration to the site of intestinal inflammation and restore immune homeostasis.

As for the other systemic T lymphocyte parameters evaluated, such as CD4+CD25+Foxp3+, CD3+CD56+, CD4+CD62+CD127+, CD4+/CD8+ ratio and CD4+CD25+, the differences between the results obtained for the two treatment arms imply that DR-6MP induces a different systemic immunological profile than that of PURINETHOL®. The results support a different mechanism of action for the two compounds.

The severity of mucosal inflammation, as assessed by colonoscopy/ileoscopy, has been touted an additional mainstay parameter for efficacy assessment in clinical trials. Moreover, the necessity for treatment to induce mucosal tissue healing in inflammatory bowel disease, in general, has become clinically relevant in light of recent reports correlating disease activity with a patient's overall risk of developing colorectal cancer. Therefore, the assessment of mucosal healing in a subset of patients willing to undergo colonoscopy/ileoscopy has been included as one of the secondary efficacy parameters in this study. Very few patients agreed to undergo colonoscopy and ileoscopy at both time points in the study; therefore, statistical analysis of evaluation of mucosal healing at week 12 relative to baseline could not be performed. Of the 4 patients who underwent this evaluation at both time points, Improvement in the total CDEIS score was observed in ⅔ subjects in the DR-6MP arm and in 1 subject in the PURINETHOL® arm. In all cases, improvement in CDEIS (i.e. mucosal healing) was correlated with reduction in CDAI scores.

Although generally regarded as a safety measure, changes in weight and BMI were included in the efficacy analysis since weight loss is a hallmark of CD. Weight and BMI increased in the DR-6MP treatment arm from baseline to each study visit and decreased in the PURINETHOL® treatment arm. A statistically significant difference between treatment arms was observed at week 8. This data supports the clinical efficacy of DR-6MP, indicating that it has a profound effect on the disease activity by preventing the weight loss that is seen in CD patients.

There were statistically significantly more AEs reported in the PURINETHOL® treatment arm compared with the DR-6MP treatment arm. The proportion of subjects with drug-related nausea, abdominal pain, decreased appetite, upper abdominal pain, asthenia and dizziness was higher in the PURINETHOL® treatment arm compared with the DR-6MP treatment arm. This indicates that DR-6MP is better tolerated than PURINETHOL®, allowing the patients a better quality of life, as also indicated by the improved IBDQ in these patients. The fewer number of AEs in the DR-6MP treatment arm may be attributed to the lack of systemic absorption of DR-6MP.

The proportion of subjects who withdrew from the study due to AEs was similar in both treatment groups, but was slightly higher in the PURINETHOL® treatment arm. The proportion of subjects who withdrew from the study due to drug-related SAEs was higher in the DR-6MP treatment arm. Most SAEs reported were transient and resolved within a few days. No deaths occurred during the study.

Leucopenia and hepatotoxicity often occur after commencing thiopurines or upon dose escalation. In this study, there was no evidence of drug-induced leucopenia in the DR-6MP group after 12 weeks of treatment. The percent of patients who had WBC within the normal range after 12 weeks of treatment was higher in the DR-6MP treatment arm (88.5%) compared with only 66.7% in the PURINETHOL® group. Patients in the PURINETHOL® group had a continuous decrease in their WBC compared to baseline during the trial with a statistically significant decrease from baseline, from week 4 onwards, while in the DR-6MP group a statistically significant decrease in WBC levels from baseline was only observed between baseline and week 12. This data supports the notion that the drug is not absorbed.

A comparable percentage of pancreatitis events occurred in both treatment arms (5% in the DR-6MP arm vs. 4.2% in the PURINETHOL® arm). Pancreatitis can either be drug-related or disease related. Moreover, unlike hepatotoxicity and leucopenia which are dose-related, pancreatitis is an idiosyncratic allergic reaction and may appear even in the presence of minute amounts of drugs in susceptible patients.

The proportion of subjects who developed drug-induced hepatotoxicity was lower for DR-6MP than for PURINETHOL® (2.5% vs. 8.3%). The increase in mean ALT at week 12, relative to baseline, was greater for the PURINETHOL® group, compared to the DR-6MP group. Bilirubin direct significantly increased in the PURINETHOL® treatment arm at week 12 compared with the DR-6MP treatment arm. The two patients from the PURINETHOL® treatment arm, who withdrew from the study prematurely due to hepatotoxicity, received DR-6MP on a “compassionate care basis”. While on DR-6MP, the previously elevated liver function tests returned to normal levels and were maintained at those levels for the period of observation—up to 7 months. Therefore, DR-6MP may provide an alternative for patients who are not able to tolerate PURINETHOL® due to hepatotoxicity.

In conclusion, DR-6MP was safer and better tolerated than PURINETHOL®.

Overall, the findings in this study complement and support each other. The CDAI difference between treatment arms at week 8 is supported by the difference between treatment arms in CRP and WBC levels and weight change, also evident at week 8. Furthermore, the increase in weight observed in the DR-6MP treatment arm corresponds to the fewer incidents of reduced appetite reported in this group of patients compared with the PURINETHOL® treatment arm.

Ongoing compassionate care data for 12 patients who completed the trial and were continued on DR-6MP have shown continued remission with no evidence of leucopenia or hepatotoxicity. This implies that the effect observed for DR-6MP at 8 weeks and onwards continues and is sustained for months.

The results of the study demonstrate that the oral DR-6MP locally delivered drug, targeted to the ileum, provides clinically effective systemic immunomodulation with fewer side-effects and a faster onset of action. Moreover, the fixed-dose regimen, independent of subject weight or side-effect profile, obviates the need for individual subject titrations and constant monitoring required for systemic immunomodulators. The better safety profile demonstrated for the DR-6MP drug suggests that treatment can be sustained over a long period of time with no concern for drug interruptions or discontinuations.

In conclusion, the data suggest that DR-6MP exerts a profound systemic clinical response without being absorbed, via exertion of a local effect in the intestinal immune system that promotes a favorable anti-inflammatory systemic immunological response. This translates to a safer, more clinically effective targeted immunomodulatory drug with less systemic immunosuppression compared to PURINETHOL®. This platform demonstrated effectiveness in Crohn's disease and may have the potential for efficacy and safety in other Crohn's-like immune-mediated disorders.

### Conclusions

### DR-6MP is

A non-absorbable immune modulator
Works locally in the intestine but has a systemic anti-inflammatory immune effect
Safer with fewer AE's, no leucopenia, less hepatotoxicity
Clinically effective
Faster clinical response
Improvement in quality-of-life
Desired weight gain
Potential to be a broad platform for other immune-mediated diseases

### REFERENCES

Baert F et al., Mucosal healing predicts sustained clinical remission in patients with early-stage Crohn's disease. Gastroenterology 2010; 138: 463-468.
Best W R et al., Development of a Crohn's disease activity index. Gastroentrology 1976; 70:439-444.
Braunwald E et al., Inflammatory Bowel Disease, Harrison's 15th edition Principles of Internal Medicine. McGraw-Hill, 2001.
Brooke B N et al., Azathioprine for Crohn's disease. Lancet 1969; 2:612-614.
Colombel J. et al., Adalimumab for Maintenance of Clinical Response and Remission in Patients with Crohn's Disease: The CHARM Trial. Gastroenterology 2007, Volume 132, Issue 1, Pages 52-65.
Crohn's & Colitis Foundation of America, What is Ulcerative Colitis? http://www.ccfa.org/what-are-crohns-and-colitis/what-is-ulcerative-colitis, accessed Apr. 16, 2015
da Cunha A P et al., Induction of immunological tolerance by oral anti-CD3. Clin Dev Immunol 2012; 2012:425021.
EMEA 2007: Guideline on the development of new medicinal products for the treatment of Crohn's disease Doc. Ref. CPMP/EWP/2284/99 Rev. 1.
Ewe K et al., Azathioprine combined with prednisolone or monotherapy with prednisolone in active Crohn's disease. Gastroenterology. 1993 105:367-72.
Fiser W., www.bio.davidson.edu/Courses/immunology/Students/spring2006/Fiser/disease.html. Crohn's Disease, 2006.
Francella A et al., The safety of 6-mercaptopurine for childbearing patients with inflammatory bowel disease: a retrospective treatment arm study. Gastroenterology 2003; 124(1):9-17.
Fuss I J et al. Disparate CD4+ lamina propria (LP) lymphokine secretion profiles in inflammatory bowel disease. Crohn's disease LP cells manifest increased secretion of IFN-gamma, whereas ulcerative colitis LP cells manifest increased secretion of IL-5. J Immunol. 1996 Aug. 1; 157(3):1261-70.
Guyatt G et al., A new measure of health status for clinical trials in inflammatory bowel disease. Gastroenterology. 1989 96:804-10.
Hanauer S B et al., et al. Maintenance infliximab for Crohn's disease: the ACCENT I randomised trial. Lancet 2002; 359(9317):1541-9.
Higgins PDR et al., An evidence-based approach to inflammatory bowel disease. Clinics in Family Practice 2004; 6(3):671-692.
Ilan Y. Oral tolerance: can we make it work? Hum Immunol. 2009 70:768-76.
Ilan Y et al, Oral administration of OKT3 monoclonal antibody to human subjects induces a dose-dependent immunologic effect in T cells and dendritic cells. J Clin Immunol 2010; 30:167-77.
Ilan Y et al., Induction of regulatory T cells decreases adipose inflammation and alleviates insulin resistance in ob/ob mice. Proc Natl Acad Sci USA 2010; 107:9765-70.
Ishikawa H et al., Inhibition of autoimmune diabetes by oral administration of anti-CD3 monoclonal antibody. Diabetes 2007; 56:2103-9.
Kim P S et al., Optimum duration of treatment with 6-mercaptopurine for Crohn's disease. American Journal of Gastroenterology 1999; 94:3254-3257.
Levy L et al., Oral immune regulation: a new mode of therapy against chronic viral infections. Recent Pat Antiinfect Drug Discov. 2007; 2:217-21.
Lewis J D et al., Inflammatory bowel disease is not associated with an increased risk of lymphoma. Gastroenterology 2001; 121(5):1080-7.
Lichtenstein G R. Remission in patients with Crohn's disease is associated with improvement in employment and quality of life and a decrease in hospitalization and surgeries. American Journal of Gastroenterology 2004; 99(1):91-96.
Lichtenstein G R et al., American Gastroenterological Association Institute Technical Review on Corticosteroids, Immunomodulators, and Infliximab in Inflammatory Bowel Disease. Gastroenterology. 2006; 130 (3):940-987.
Mantzaris G J et al., Azathioprine is superior to budesonide in achieving and maintaining mucosal healing and histologic remission in steroid-dependent Crohn's disease. Inflamm Bowel Dis. 2009 March; 15(3):375-82.
Mizrahi M et al., The gut mucosa as a site for induction of regulatory T-cells. Curr Pharm Des. 2009; 15:1191-202.
Ochi H et al., Oral CD3-specific antibody suppresses autoimmune encephalomyelitis by inducing CD4+CD25-LAP+ T cells. Nature Medicine, Volume 12 Number 6, June 2006.
Pineton de Chambrun G et al., Medscape. Clinical implications of mucosal healing for the management of IBD. Nat Rev Gastroenterol Hepatol. 2009 Dec. 1.
Present D H et al., Treatment of Crohn's disease with 6-mercaptopurine: a long-term, randomized, double-blind study. The New England Journal of Medicine 1980; 302:981-7.
Roncarolo G et al., Regulatory T-cell immunotherapy for tolerance to self antigens and alloantigens in humans. Nature Reviews Immunology 7, 585-598, August 2007.
Rutgeerts P J. Review article: the limitations of corticosteroid therapy in Crohn's disease Aliment Pharmacol Ther. 2001 October; 15(10):1515-25.
Rutgeerts P J. An historical overview of the treatment of Crohn's disease: why do we need biological therapies? Rev Gastroenterol Disord. 2004; 4 Suppl 3:S3-9.
Rutgeerts P et al., Scheduled maintenance treatment with infliximab is superior to episodic treatment for the healing of mucosal ulceration associated with Crohn's disease. Gastrointest Endosc. 2006; 63:433-442.33.
Sandborn W J et al., A review of activity indices and efficacy endpoints for clinical trials of medical therapy in adults with Crohn's disease. Gastroenterology 2002 February; 122(2):512-30.
Sandborn W J et al., Natalizumab Induction and Maintenance Therapy for Crohn's Disease. N. Engl J Med. 2007; 357:239-50.
Schurmann G M et al., Increased expression of cell adhesion molecule P-selectin in active inflammatory bowel disease. Gut 1995; 36: 411-418.
Shevach E M. From Vanilla to 28 Flavors: Perspective Multiple Varieties of T Regulatory Cells. Immunity 25, 195-201, August 2006.
Simms L et al, Budesonide for maintenance of remission in Crohn's disease. Cochraine Database of Systematic Reviews 2001, Issue 1.
Sninsky C A. Altering the natural history of Crohn's disease? Inflamm Bowel Dis. 2001 May; 7 Suppl 1:S34-9.
Sostegni R et al., Crohn's disease: monitoring disease activity. Aliment Pharmacol Ther 2003; 17 (Suppl. 2): 11-17.
Steinhart A et al., Corticosteroids for maintenance of remission in Crohn's disease. Cochraine Database of Systematic Reviews 2003, Issue 4.
Summers R W et al., National Cooperative Crohn's Disease Study: results of drug treatment. Gastroenterology 1979; 77(4 Pt2):847-69.
Targan S R et al. A short-term study of chimeric monoclonal antibody cA2 to tumor necrosis factor alpha for Crohn's disease. Crohn's Disease cA2 Study Group. N Engl J Med. 1997 Oct. 9; 337(15):1029-1035.
Travis S P L et al. Review article: defining remission in ulcerative colitis. Aliment Pharmacol Ther 2011; 34: 113-124
Vermeire S et al., Recent advances in clinical practice Laboratory markers in IBD: useful, magic, or unnecessary toys? Gut 2006; 55:426-431.
Weiner H L et al., Oral tolerance. Immunol Rev. 2011; 241:241-59.
Wu H Y et al., Suppression of murine SLE by oral anti-CD3: inducible CD4+CD25-LAP+ regulatory T cells control the expansion of IL-17+ follicular helper T cells. Lupus 2009; 18:586-96.

What is claimed is:

1. A method of treating a human patient suffering from Crohn's disease (CD) or ulcerative colitis (UC) who did not experience a clinical response to previous thiopurine administration, comprising periodically administering to the human patient a delayed release pharmaceutical composition comprising a pharmaceutically acceptable carrier and an amount of 6-mercaptopurine (6-MP) effective to treat the human patient.

2. The method of claim 1, wherein the patient did not experience a clinical response after 4 weeks of previous thiopurine administration, or after 12 weeks of previous thiopurine administration.

3. The method of claim 1, wherein the delayed release pharmaceutical composition is administered daily for a period of time of up to 12 weeks; wherein the delayed release pharmaceutical composition is administered daily for a period of time of up to 8 weeks; wherein the delayed release pharmaceutical composition is administered daily and the maximal clinical response is achieved 8 weeks from the beginning of administration; or wherein the maximal clinical response is achieved 8 weeks from the beginning of administration.

4. (canceled)

5. The method of claim 1, wherein the patient is suffering from CD and the Crohn's Disease Activity Index (CDAI) score of the patient is about 220 or more before the treatment; or is about 220 to about 450 before the treatment.

6. The method of claim 1, wherein the patient is suffering from CD and the administration of the delayed release pharmaceutical composition to the patient results in a clinical response, in remission of CD, in mucosal healing, or in an improved side effect profile compared to administration of an immediate release formulation of 6-MP; or
wherein the patient is suffering from UC and the administration of the delayed release pharmaceutical composition to the patient results in remission of UC.

7-9. (canceled)

10. The method of claim 1, wherein the delayed release pharmaceutical composition administered to the patient contains 40 mg to 120 mg of 6-MP; 40 mg to 100 mg of 6-MP; 60 mg to 80 mg of 6-MP; 80 mg of 6-MP; or 120 mg of 6-MP.

11. The method of claim 1, wherein the delayed release pharmaceutical composition is administered once per day; or wherein the delayed release pharmaceutical composition is administered once per day and the administration is oral administration.

12. A method of treating a human patient suffering from Crohn's disease (CD) or ulcerative colitis (UC) who has experienced an adverse event in response to previous administration of thiopurine, comprising periodically administering to the human patient a delayed release pharmaceutical composition comprising a pharmaceutically acceptable carrier and an amount of 6-mercaptopurine (6-MP) effective to treat the human patient, wherein the adverse event is other than raised liver function test results (LFTs) if the administered thiopurine is 6-MP.

13. The method of claim 12, wherein the delayed release pharmaceutical composition is administered daily for a period of time of up to 12 weeks; wherein the delayed release pharmaceutical composition is administered daily for a period of time of up to 8 weeks; wherein the delayed release pharmaceutical composition is administered daily and the maximal clinical response is achieved 8 weeks from the beginning of administration; or wherein the maximal clinical response is achieved 8 weeks from the beginning of administration.

14. (canceled)

15. The method of claim 12, wherein the patient is suffering from CD and the Crohn's Disease Activity Index (CDAI) score of the patient is about 220 or more before the treatment; or is about 220 to about 450 before the treatment.

16. The method of claim 12, wherein the patient is suffering from CD and the administration of the delayed release pharmaceutical composition to the patient results in a clinical response, in remission of CD, in mucosal healing, or in an improved side effect profile compared to administration of an immediate release formulation of 6-MP; or
wherein the patient is suffering from UC and the administration of the delayed release pharmaceutical composition to the patient results in remission of UC.

17-19. (canceled)

20. The method of claim 12, wherein the delayed release pharmaceutical composition administered to the patient contains 40 mg to 120 mg of 6-MP; 40 mg to 100 mg of 6-MP; 60 mg to 80 mg of 6-MP; 80 mg of 6-MP; or 120 mg of 6-MP.

21. The method of claim 12, wherein the delayed release pharmaceutical composition is administered once per day; or wherein the delayed release pharmaceutical composition is administered once per day and the administration is oral administration.

22. A method of treating a human patient suffering from Crohn's disease (CD) or ulcerative colitis (UC) who is being administered an antibiotic, who is being administered 5-aminosalisylic acid (5-ASA), or who is receiving administration of a steroid and who is steroid-dependent, comprising adjunctively periodically administering to the human patient a delayed release pharmaceutical composition comprising a pharmaceutically acceptable carrier and an amount of 6-mercaptopurine (6-MP) effective to treat the human patient.

23. The method of claim 22, wherein the pharmaceutical composition is administered daily for a period of time of up to 12 weeks; wherein the delayed release pharmaceutical composition is administered daily for a period of time of up to 8 weeks; wherein the delayed release pharmaceutical composition is administered daily and the maximal clinical response is achieved 8 weeks from the beginning of administration; or wherein the maximal clinical response is achieved 8 weeks from the beginning of administration.

24. (canceled)

25. The method of claim 22, wherein the patient is receiving administration of a steroid; and
wherein the steroid is an oral steroid; wherein the steroid is a low-dose oral steroid; wherein the steroid is prednisolone; wherein the steroid is prednisolone and the patient is receiving ≦15 mg of prednisone per day; wherein the steroid is budesonide; or wherein the steroid is budesonide and the patient is receiving 6 mg of budesonide per day.

26. The method of claim 22, wherein the CDAI score of the patient is about 220 or more before beginning administration of the delayed release pharmaceutical composition; or is about 220 to about 450 before beginning administration of the delayed release pharmaceutical composition.

27. The method of claim 22, wherein the administration of the delayed release pharmaceutical composition to the patient results in a clinical response, in remission of CD, in mucosal healing, or in an improved side effect profile compared to administration of an immediate release formulation of 6-MP; or
wherein the patient is suffering from UC and the administration of the delayed release pharmaceutical composition to the patient results in remission of UC.

28-30. (canceled)

31. The method of claim 22, wherein the delayed release pharmaceutical composition administered to the patient contains 40 mg to 120 mg of 6-MP; 40 mg to 100 mg of 6-MP; 60 mg to 80 mg of 6-MP; 80 mg of 6-MP; or 120 mg of 6-MP.

32. The method of claim 22, wherein the dose of 6-MP is administered once per day; wherein the delayed release pharmaceutical composition is administered once per day and the administration is oral administration; or wherein the amount of the delayed release pharmaceutical composition and the amount of the steroid, 5-ASA or antibiotic when taken together is more effective to treat the patient than when each agent is administered alone.

33-46. (canceled)
